(19) United States
(12) Patent Application Publication
(10) Pub. No.: US 2015/0038435 A1
(43) Pub. Date: Feb. 5, 2015

(54) N-TERMINALLY MODIFIED OLIGOPEPTIDES AND USES THEREOF

(71) Applicant: Novo Nordisk A/S, Bagsvaerd (DK)

(72) Inventor: Frantisek Hubalek, Herlev (DK)

(21) Appl. No.: 14/380,914

(22) PCT Filed: Mar. 1, 2013
(86) PCT No.: PCT/EP2013/054177

§ 371 Date: Aug. 25, 2014

Related U.S. Application Data
(60) Provisional application No. 61/625,896, filed on Apr. 18, 2012.

(30) Foreign Application Priority Data
Mar. 1, 2012 (EP) 12157616.9

(51) Int. Cl. C07K 7/06

(52) U.S. Cl. CPC C07K 7/06
USPC 514/21.6; 530/328

(57) ABSTRACT

The present invention is related to N-terminally fatty acid modified peptides or oligopeptides and pharmaceutical compositions comprising such.

### TECHNICAL FIELD

The present invention is related to N-terminally fatty acid modified peptides or oligopeptides and pharmaceutical compositions comprising such.

### BACKGROUND

The oral route is by far the most widely used route for drug administration. Administration of peptides and proteins is however often limited to parenteral routes rather than the preferred oral administration due to several barriers such as enzymatic degradation in the gastrointestinal (GI) tract and intestinal mucosa, drug efflux pumps, insufficient and variable absorption from the intestinal mucosa, as well as first pass metabolism in the liver.

To overcome this barrier, inhibitors of protease degradation are commonly included in oral pharmaceutical compositions and/or the active ingredients are stabilized towards proteolytic degradation. There are many protease inhibitors available in the public domain. However, many of them are toxic or allergenic, including soya bean trypsin inhibitor (Kunitz type, SBTI) and therefore not applicable for chronic administration.

Further, protease inhibitors described in the public domain such as SBTI have the disadvantage to be chemically unstable in liquid lipid and surfactant based pharmaceutical compositions such as self-nanoemulsifying drug delivery systems (SNEDDS). Aldehyde and peroxide impurities present in these excipients are known to react with amino-groups of peptides and proteins and will therefore negatively impact the shelf life.

Another disadvantage of SBTI is its low solubility in lipid pharmaceutical compositions which results in physically unstable compositions.

There is thus a need for new protease inhibitors with improved characteristics.

### SUMMARY

The present invention is related to an N-terminally acylated peptide or oligopeptide having the structure

Cx-Aaa10-Aaa9-Aaa8-Aaa7-Aaa6-Aaa5-Aaa-4-Aaa3-Aaa2-Aaa1-OH; SEQ ID No: 1
Chem I
   where Cx is a fatty acid with a length between 6 and 20 carbons, and wherein Aaa1 is an aromatic amino acid; Aaa2 is any amino acid except Lys or Asp; Aaa3 is any amino acid; and Aaa-4-10 is any amino acid or absent.

In an aspect of the invention, an N-terminally acylated peptide or oligopeptide is an inhibitor of proteolytic activity in an extract from the gastrointestinal tract (GI tract).

In an aspect of the invention, an N-terminally acylated peptide or oligopeptide according to any one of the preceding claims, which is an inhibitor of proteolytic activity such as proteolytic activity of trypsin, chymotrypsin, elastase, carboxypeptidase and/or aminopeptidase.

In an aspect of the invention, an N-terminally acylated peptide or oligopeptide according to any one of the preceding claims is an absorption enhancer.

The invention is also related to oral pharmaceutical compositions comprising an N-terminally acylated peptide or oligopeptide of the invention and further a pharmaceutically active ingredient. In an aspect of the invention, the further pharmaceutically active ingredient is a peptide or protein. In an aspect, an oral pharmaceutical composition of the invention is a liquid or semi-liquid composition. In an aspect, an oral pharmaceutical composition of the invention is a solid composition.

The invention may also solve further problems that will be apparent from the disclosure of the exemplary aspects.

### DESCRIPTION

The present invention is related to N-terminally fatty acid modified peptides or oligopeptides. In one aspect, the fatty acid has a length between 6-20 carbon atoms. In one aspect the peptide or oligopeptide has between 2-10 amino acids. In one aspect the peptide or oligopeptide has between 2-8 amino acids. In one aspect the peptide or oligopeptide has between 3-8 amino acids. In one aspect the peptide or oligopeptide has between 3-6 amino acids.

In one aspect, the invention is related to an N-terminally acylated peptide or oligopeptide having the structure

Cx-Aaa10-Aaa9-Aaa8-Aaa7-Aaa6-Aaa5-Aaa-4-Aaa3-Aaa2-Aaa1-OH; SEQ ID No: 1
Chem I
   where Cx is a fatty acid with a length between 6 and 20 carbons, and wherein Aaa1 is an aromatic amino acid; Aaa2 is any amino acid except Lys or Asp; Aaa3 is any amino acid; and Aaa-4-10 is any amino acid or absent.

In one aspect, the invention is related to an N-terminally acylated peptide or oligopeptide having the structure

Cx-Aaa10-Aaa9-Aaa8-Aaa7-Aaa6-Aaa5-Aaa-4-Aaa3-Aaa2-Aaa1-OH; SEQ ID No: 1
Chem I
   where Cx is a fatty acid with a length between 6 and 20 carbons, and wherein Aaa1 is an aromatic amino acid; Aaa2 is any amino acid except Lys or Asp; Aaa3 is Trp, Tyr, Phe, Arg, Lys or His; Aaa-4-9 is any amino acid or absent, and Aaa10 is Leu, Thr, Lys, Arg or His or absent.

In one aspect of the invention Cx is a fatty acid with a length between 12 and 20 carbons, in one aspect Cx is a fatty acid with a length between 12 and 16 carbons.

The term “fatty acid” refers to aliphatic monocarboxylic acids having 6 carbon atoms or more, it is preferably unbranched, and/or even numbered, and it may be saturated or unsaturated. “Fatty acids” of the invention are thus understood as saturated monocarboxylic acids e.g. of the formula CH3-(CH2)n-COOH or CH3-(CH2)n-CH(CH3)-(CH2)n-COOH, or unsaturated monocarboxylic acids, e.g. of formula CH3-(CH2)n-CH═CH—(CH2)n-COOH, which do not comprise any heteroatoms. When reacted with a peptide or polypeptide, the carboxylic acid group of the fatty acid typically reacts with a nitrogen or another reactive group of the (poly)peptide and forms a fatty acid modified (poly)peptide of the formula R—C(═O)-(poly)peptide where R is an alkane or alkene.

Herein, the term “amino acid residue” is an amino acid from which, formally, a hydroxy group has been removed from a carboxy group and/or from which, formally, a hydrogen atom has been removed from an amino group.

The term “amino acid” includes proteogenic amino acids (encoded by the genetic code, including natural amino acids, and standard amino acids), as well as non-proteogenic (not found in proteins, and/or not coded for in the standard genetic code), and synthetic amino acids. Thus, the amino acids of an N-terminally acylated peptide or oligopeptide of the invention may be selected from the group of proteinogenic amino acids, non-proteinogenic amino acids, and/or synthetic amino acids. In one aspect the amino acids are selected from one or more of the group consisting of proteogenic amino acids, D-form of proteogenic amino acids, OEG ([2-(2-aminoethoxy)ethoxy]ethylcarbonyl), γGlu and βAsp. In one aspect the amino acids are selected from one or more of the group consisting of proteogenic amino acids, OEG ([2-(2-aminoethoxy)ethoxy]ethylcarbonyl), γGlu and βAsp. In one aspect the amino acids are proteogenic amino acids.

Herein, the following abbreviations are used: “OEG” for 8-amino-3,6-dioxaoctanoic acid; “gamma-Glu” (or “gGlu, “γGlu” or “γ-Glu”) for gamma-glutamic acid; beta-Asp (or “bAsp”, “β-Asp” or “βAsp”) for beta-aspartic acid; and epsilon-Lys (or “eLys” or “e-Lys”, “εLys” or “ε-Lys”) for epsilon-lysine.

Glutamic acid and aspartic acid by nature each have two carboxyl (—COOH) groups and may thus react in each of these groups. The carboxyl group on the α-carbon is referred to as the α carboxyl group, the side chain carboxyl of aspartic acid is referred to as the β carboxyl group and the side chain carboxyl of glutamic acid is referred to as the γ carboxyl group.

For illustration, a di-radical of glutamic acid (a γGlu di-radical) is illustrated in Chem. 1:

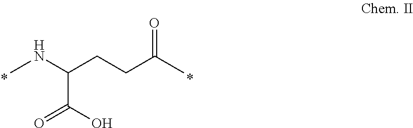

In Chem. II the alpha-amino and the gamma-carboxyl groups are presented as radicals. Chem. II may thus also be referred to as gamma-Glu, or briefly γGlu, due to the fact that it is the gamma carboxy group of glutamic acid which is here used for connection to another amino acid residue. The amino group of Glu in turn forms an amide bond with the carboxy group of yet another amino acid or the carboxy group of the fatty acid. Similarly, aspartic acid may be referred to as beta-Asp, or briefly βAsp, when the beta carboxy group of aspartic acid is used for connection to another amino acid residue or the carboxy group of the fatty acid and lysine may be referred to as epsilon-Lys, or briefly εLys, when the epsilon amino group of lysine is used for connection to another amino acid residue or the carboxy group of the fatty acid.

Non-limiting examples of amino acids which are not encoded by the genetic code are gamma-carboxyglutamate, ornithine, and phosphoserine. Non-limiting examples of synthetic amino acids are the D-isomers of the amino acids such as D-alanine and D-leucine, Aib (α-aminoisobutyric acid), β-alanine, des-amino-histidine (desH, alternative name imidazopropionic acid, abbreviated Imp) and OEG ([2-(2-aminoethoxy)ethoxy]ethylcarbonyl).

The term “aromatic amino acid” is herein used for an amino acid that includes an aromatic ring. Non-limiting examples of aromatic amino acids include phenylalanine, tryptophan, histidine, tyrosine and thyroxine (also named 3,5,3′,5′-tetraiodothyronine).

The term “basic amino acid” is herein used for an amino acid which is polar and positively charged at pH values below its pKa, i.e. an amino acid that includes a side chain that is basic at neutral pH. Non-limiting examples of basic amino acids include arginine (Arg), lysine (Lys), and histidine (His).

It has surprisingly been found that the N-terminally fatty acid modified peptides or oligopeptides of the invention function as protease inhibitors when used in oral compositions.

It has thus surprisingly been found that the N-terminally fatty acid modified peptides or oligopeptides of the invention bind to proteolytic enzymes in such a way to interfere with degradation of peptides/proteins.

In general compounds can bind to proteolytic enzymes at many different sites, however, it is only binding that interferes with the function of proteolytic enzymes that is of interest when searching for inhibitors of proteolysis. The best way to look for inhibitors is to examine the effect of the presence of the potential inhibitor on the enzymatic reaction catalyzed by the protease in question. Enzyme kinetics describes several possibilities for a compound to inhibit an enzyme as known to the person skilled in the art. Enzyme inhibition can be, for example, competitive, non-competitive, mixed. Procedures for distinguishing different kinds of enzyme inhibition were previously described in many scientific articles and numerous textbooks, for example, Fundamentals of Enzyme Kinetics by Athel Cornish-Bowden ISBN-13: 978-3527330744. In addition to enzyme kinetics, interactions of proteolytic enzymes with their inhibitors are commonly examined by many different methods, for example, x-ray crystallography, NMR spectroscopy, numerous spectroscopy techniques (fluorescence, circular dischroism, UV-VIS), mass spectrometry, calorimetry, etcetera as known to the person skilled in the art. Compounds can also strongly bind to an enzyme but not affect the rate of the catalyzed reaction.

In the process of developing the N-terminally acylated (oligo)peptides of the invention we have found that Kᵢ for the interaction between the N-terminally acylated (oligo)peptides of the invention and chymotrypsin depends on the substrate used in the assay. For example, the Kᵢ for compound from example 34 was ˜130 μM when N-succinyl-Ala-Ala-Pro-Phe-p-Nitroanilide was used as substrate and it was a case of competitive inhibition (example 202), while Kᵢ˜15 μM was found when A14E, B25H, B29K(N(eps)Octadecanedioyl-γGlu-OEG-OEG), desB30 human insulin was used as substrate (example 201) and this was a case of mixed inhibition. These results are consistent with two binding sites for compound 34 on chymotrypsin; “high” affinity (˜15 μM) binding site interferes with insulin degradation but not with N-succinyl-Ala-Ala-Pro-Phe-p-Nitroanilide degradation. This site could be present close to the active site but not involving the P1-P4 sites that are needed to bind and degrade N-succinyl-Ala-Ala-Pro-Phe-p-Nitroanilide, and “low” affinity (˜150 μM) binding site interferes with N-succinyl-Ala-Ala-Pro-Phe-p-Nitroanilide degradation and does very likely involve P1-P4 sites of chymotrypsin.

It will be appreciated that a person of ordinary skill will choose the appropriate substrate when testing oligopeptides of the invention. Often commercially available chromogenic/fluorogenic substrates are used for enzyme assays as these are easy to use and amenable to high throughput setup. For example chymotrypsin activity can be monitored using Suc-Ala-Ala-Pro-Phe-p-nitro anilide (A sensitive new substrate for chymotrypsin. DelMar, E. G., et al. Anal. Biochem. 99, 316, (1979); Mapping the extended substrate binding site of cathepsin G and human leukocyte elastase. Studies with peptide substrates related to the alpha 1-protease inhibitor reactive site. Nakajima, K., et al. J. Biol. Chem. 254, 4027, (1979)), similarly trypsin activity can be followed for example by using Benzoyl-Phe-Val-Arg-p-nitroanilide (Substrates for determination of trypsin, thrombin and thrombin-like enzymes. Svendsen, L., et al. Folia Haematol. Int. Mag. Klin. Morphol. Blutforsch. 98, 446, (1972); Assay of coagulation proteases using peptide chromogenic and fluorogenic substrates. Lottenberg, R., et al. Meth. Enzymol. 80, 341, (1981)). There is, however, a possibility that enzyme inhibitors that do not bind directly to the active site of the tested enzyme (for example inhibitors that can formally be described as non-competitive or mixed inhibitors) will be missed using these chromogenic/fluorogenic substrates. By using chromogenic/fluorogenic substrates, inhibition by a compound that binds adjacent to the active site of the enzyme and interferes with binding of the relevant substrate which is typically larger than the chromogenic/fluorogenic substrates, may not be seen. The skilled person will know how to verify that the enzyme inhibition observed with the chromogenic/fluorogenic substrate is of the same type and magnitude as observed for the “real” substrate, i.e. as observed for insulin in the case of oral delivery of insulin or for GLP-1 in the case of oral delivery of GLP-1. Alternatively, a custom substrate structurally similar to the “real” substrate may be designed for example by utilizing Förster resonance energy transfer (FRET, as described for example in Examples 198 and 199). Based on his knowledge about chromogenic, fluorogenic and custom made substrates, the skilled person will know how to first screen or verify the screening results with the relevant substrate before selecting the final substrate for screening.

In one aspect, the N-terminally fatty acid modified peptides or oligopeptides of the invention are suitable for use in oral pharmaceutical compositions. In one aspect of the invention, the N-terminally fatty acid modified peptides or oligopeptides of the invention are fully biodegradable to amino acids and fatty acids when used in oral pharmaceutical compositions, where biodegradable means degradable in vivo. I.e., in one aspect the N-terminally acylated (oligo)peptides of the invention are fully degraded in vivo. In one aspect, the N-terminally fatty acid modified peptides or oligopeptides are suitable for use in liquid or semi-liquid oral compositions such as e.g. SNEDDS compositions. In an aspect of the invention, the N-terminally fatty acid modified peptides or oligopeptides are suitable for use in solid (oral) pharmaceutical compositions, also known as solid (oral) dosage forms, such as e.g. tablets in powder form which are pressed or compacted from a powder into a solid dose which is optionally further coated. In an aspect, the N-terminally fatty acid modified peptides or oligopeptides are suitable for use in a tablet. In an aspect, the N-terminally fatty acid modified peptides or oligopeptides are suitable for use in a capsule.

In one aspect an N-terminally fatty acid modified peptide or oligopeptide according to the invention stabilizes the active ingredient against degradation by one or more proteolytic enzymes.

The binding constant, Kᵢ, for binding of an N-terminally fatty acid modified peptide or oligopeptide of the invention to a proteolytic enzyme may be used as a measure of how well the N-terminally fatty acid modified peptide or oligopeptide of the invention stabilizes the active ingredient against degradation by said proteolytic enzyme.

In one aspect of the invention, Kᵢ, when binding an N-terminally fatty acid modified peptide or oligopeptide of the invention to chymotrypsin is in the range from 100 nM to 100 μM. The lower Kᵢ the stronger inhibition is observed for a given concentration of the N-terminally acylated peptide or oligopeptide of the invention. In one aspect of the invention, Kᵢ, when binding an N-terminally fatty acid modified peptide or oligopeptide of the invention to chymotrypsin is in the range from 500 μM to 100 nM, from 50 μM to 100 nM, from 10 μM to 100 nM. In one aspect of the invention, Kᵢ, when binding an N-terminally fatty acid modified peptide or oligopeptide of the invention to trypsin is in the range from 500 μM to 100 nM, from 100 μM to 100 nM, from 50 μM to 100 nM, from 10 μM to 100 nM. In one aspect of the invention, Kᵢ, when binding an N-terminally fatty acid modified peptide or oligopeptide of the invention to elastase is in the range from 500 μM to 100 nM, from 100 μM to 100 nM, from 50 μM to 100 nM, from 10 μM to 100 nM.

EC₅₀, i.e. the half maximal effective concentration, of an N-terminally fatty acid modified peptide or oligopeptide of the invention is a measure of the concentration which induces a response halfway between the baseline and maximum after some specified exposure time and may be used as a measure of how well the N-terminally fatty acid modified peptide or oligopeptide of the invention stabilizes the active ingredient against degradation by said proteolytic enzyme. The EC₅₀ value depends on the experimental conditions and the same conditions must thus be used when comparing EC₅₀ values. However, provided that additional parameters such as Kₘ (Michaelis constant) for the given reaction are known, the EC₅₀ values can be converted to Kᵢ values (Brandt, R. B et al Biochemical medicine and metabolic biology 37, 344-349 (1987)).

In one aspect an N-terminally fatty acid modified peptide or oligopeptide according to the invention stabilizes the active ingredient against degradation by one or more enzymes selected from the group consisting of: chymotrypsin, trypsin, Insulin-Degrading Enzyme (IDE), elastase, carboxypeptidases, aminopeptidases and cathepsin D. In a further aspect an N-terminally fatty acid modified peptide or oligopeptide according to the invention stabilizes the active ingredient against degradation by one or more enzymes selected from the group consisting of: chymotrypsin, trypsin and elastase. In a yet further aspect an N-terminally fatty acid modified peptide or oligopeptide according to the invention stabilizes the active ingredient against degradation by one or more enzymes selected from: chymotrypsin and trypsin. In a yet further aspect an N-terminally fatty acid modified peptide or oligopeptide according to the invention stabilizes the active ingredient against degradation by chymotrypsin. In a yet further aspect an N-terminally fatty acid modified peptide or oligopeptide according to the invention stabilizes the active ingredient against degradation by trypsin. In a yet further aspect an N-terminally fatty acid modified peptide or oligopeptide according to the invention stabilizes the active ingredient against degradation by elastase. In one aspect an N-terminally fatty acid modified peptide or oligopeptide according to the invention stabilizes the active ingredient against degradation in an extract from the gastrointestinal tract (GI extract), i.e. a mixture of enzymes such as tissue extracts from the gastrointestinal tract.

A “protease”, “protease enzyme” or “proteolytical enzyme” is a digestive enzyme which degrades proteins and peptides and which is found in various tissues of the human body such as e.g. the stomach (pepsin), the intestinal lumen (chymotrypsin, trypsin, elastase, carboxypeptidases, etc.) or mucosal surfaces of the GI tract (aminopeptidases, carboxypeptidases, enteropeptidases, dipeptidyl peptidases, endopeptidases, etc.), the liver (Insulin degrading enzyme, cathepsin D etc), and in other tissues.

T½ may be determined as a measure of the proteolytical stability of the active ingredient obtained by addition of an N-terminally fatty acid modified peptide or oligopeptide according to the invention to an oral composition, wherein the proteolytical stability is towards protease enzymes such as chymotrypsin, trypsin and/or elastase or towards a mixture of enzymes such as tissue extracts (from liver, kidney, duodenum, jejunum, ileum, colon, stomach, etc.). In one aspect of the invention T½ is increased relative to an oral composition without the N-terminally fatty acid modified peptide or oligopeptide of the invention. In a further aspect T½ is increased at least 2-fold relative to an oral composition without the N-terminally fatty acid modified peptide or oligopeptide of the invention. In a yet further aspect T½ is increased at least 3-fold relative to an oral composition without the N-terminally fatty acid modified peptide or oligopeptide of the invention. In a yet further aspect T½ is increased at least 4-fold relative to an oral composition without the N-terminally fatty acid modified peptide or oligopeptide of the invention. In a yet further aspect T½ is increased at least 5-fold relative to an oral composition without the N-terminally fatty acid modified peptide or oligopeptide of the invention. In a yet further aspect T½ is increased at least 10-fold relative to an oral composition without the N-terminally fatty acid modified peptide or oligopeptide of the invention. In a yet further aspect T½ is increased at least 50-fold relative to an oral composition without the N-terminally fatty acid modified peptide or oligopeptide of the invention. In a yet further aspect T½ is increased at least 100-fold relative to an oral composition without the N-terminally fatty acid modified peptide or oligopeptide of the invention.

In one aspect, T½ is determined as a measure of the proteolytical stability obtained by addition of an N-terminally fatty acid modified peptide or oligopeptide according to the invention to an aqueous solution comprising the active ingredient, wherein the proteolytical stability is towards protease enzymes such as chymotrypsin, trypsin and/or elastase or towards a mixture of enzymes such as tissue extracts (from liver, kidney, duodenum, jejunum, ileum, colon, stomach, etc.). In one aspect of the invention T½ is increased relative to an aqueous solution comprising the active ingredient without the N-terminally fatty acid modified peptide or oligopeptide of the invention. In a yet further aspect T½ is increased at least 2-fold relative to an aqueous solution comprising the active ingredient without the N-terminally fatty acid modified peptide or oligopeptide of the invention. In a yet further aspect T½ is increased at least 3-fold relative to an aqueous solution comprising the active ingredient without the N-terminally fatty acid modified peptide or oligopeptide of the invention. In a yet further aspect T½ is increased at least 4-fold relative to an aqueous solution comprising the active ingredient without the N-terminally fatty acid modified peptide or oligopeptide of the invention. In a yet further aspect T½ is increased at least 5-fold relative to an aqueous solution comprising the active ingredient without the N-terminally fatty acid modified peptide or oligopeptide of the invention. In a yet further aspect T½ is increased at least 10-fold relative to an aqueous solution comprising the active ingredient without the N-terminally fatty acid modified peptide or oligopeptide of the invention. In a yet further aspect T½ is increased at least 50-fold relative to an aqueous solution comprising the active ingredient without the N-terminally fatty acid modified peptide or oligopeptide of the invention. In a yet further aspect T½ is increased at least 100-fold relative to an aqueous solution comprising the active ingredient without the N-terminally fatty acid modified peptide or oligopeptide of the invention.

It has surprisingly been found that the N-terminally fatty acid modified peptides or oligopeptides of the invention may also function as absorption enhancers when used in oral compositions.

It has thus surprisingly been found that the N-terminally fatty acid modified peptides or oligopeptides of the invention may improve the absorption of an active ingredient when included in an oral pharmaceutical composition.

The terms “permeation enhancer” and “absorption enhancer” are herein used interchangably and refer to biologicals or chemicals that promote the intestinal absorption of drugs i.e. increasing permeability of poorly permeable pharmaceuticals and thereby improve oral drug bioavailability. Delivery of a pharmaceutical by oral route is thus predominantly restricted by pre-systemic degradation and poor penetration across the gut wall. The major challenge in the oral drug delivery is the development of novel dosage forms to endorse absorption of poorly permeable drugs across the intestinal epithelium.

To asses whether a compound is an absorption enhancer, such compound is typically examined in at least one of the assays known in the art to measure absorption of a drug or a model compound across a cell layer. Nonlimiting examples of such assays are Caco-2 cell assay (for example as described in the examples) or Ussing chamber assay (as described for example in Fetih G, Habib F, Okada N, Fujita T, Attia M, Yamamoto A. Nitric oxide donors can enhance the intestinal transport and absorption of insulin and [Asu(1,7)]-eel calcitonin in rats. J Control Release. 2005; 106(3):287-97; or Shimazaki T, Tomita M, Sadahiro S, Hayashi M, Awazu S. Absorption-enhancing effects of sodium caprate and palmitoyl carnitine in rat and human colons. Dig Dis Sci. 1998; 43(3):641-5; or Petersen S B, Nolan G, Maher S, Rahbek U L, Guldbrandt M, Brayden D J. Evaluation of alkylmaltosides as intestinal permeation enhancers: comparison between rat intestinal mucosal sheets and Caco-2 monolayers. Eur J Pharm Sci. 2012; 47(4):701-12.). In one aspect of the invention, absorption enhancement is increased relative to an aqueous solution comprising the active ingredient without the N-terminally fatty acid modified peptide or oligopeptide of the invention. In a further aspect, absorption enhancement is increased at least 1.5-fold relative to an aqueous solution comprising the active ingredient without the N-terminally fatty acid modified peptide or oligopeptide of the invention. In a yet further aspect, absorption enhancement is increased at least 2-fold relative to an aqueous solution comprising the active ingredient without the N-terminally fatty acid modified peptide or oligopeptide of the invention. In a yet further aspect, absorption enhancement is increased at least 3-fold relative to an aqueous solution comprising the active ingredient without the N-terminally fatty acid modified peptide or oligopeptide of the invention. In a yet further aspect, absorption enhancement is increased at least 4-fold relative to an aqueous solution comprising the active ingredient without the N-terminally fatty acid modified peptide or oligopeptide of the invention. In a yet further aspect, absorption enhancement is increased at least 5-fold relative to an aqueous solution comprising the active ingredient without the N-terminally fatty acid modified peptide or oligopeptide of the invention. In a yet further aspect, absorption enhancement is increased at least 6-fold relative to an aqueous solution comprising the active ingredient without the N-terminally fatty acid modified peptide or oligopeptide of the invention. In a yet further aspect, absorption enhancement is increased at least 7-fold relative to an aqueous solution comprising the active ingredient without the N-terminally fatty acid modified peptide or oligopeptide of the invention. In a yet further aspect, absorption enhancement is increased at least 8-fold relative to an aqueous solution comprising the active ingredient without the N-terminally fatty acid modified peptide or oligopeptide of the invention. In a yet further aspect, absorption enhancement is increased at least 9-fold relative to an aqueous solution comprising the active ingredient without the N-terminally fatty acid modified peptide or oligopeptide of the invention. In a yet further aspect, absorption enhancement is increased at least 10-fold relative to an aqueous solution comprising the active ingredient without the N-terminally fatty acid modified peptide or oligopeptide of the invention.

In one aspect, an N-terminally fatty acid modified peptides or oligopeptides of the invention is selected from the group consisting of:
N-dodecanoyl-Ala-Ala-Pro-Phe-OH
N-dodecanoyl-DAla-DAla-DPro-DPhe-OH
N-tetradecanoyl-Ala-Ala-Pro-Phe-OH
N-dodecanoyl-γGlu-OEG-Ala-Ala-Pro-Phe-OH
N-tetradecanoyl-Ala-Ala-Pro-DPhe-OH
N-tetradecanoyl-βAla-Ala-Pro-Phe-OH
N-dodecanoyl-OEG-OEG-DPhe-OH
N-dodecanoyl-Ala-Ala-Ala-Ala-Pro-Phe-OH
N-dodecanoyl-γGlu-Ala-Pro-Phe-OH
N-tetradecanoyl-γGlu-Ala-Pro-Phe-OH
N-dodecanoyl-Ala-Ala-Pro-Trp-OH
N-eicosanoyl-Ala-Ala-Pro-Phe-OH
N-hexadecanoyl-Ala-Ala-Pro-Phe-OH
N-hexadecanoyl-γGlu-Ala-Ala-Pro-Phe-OH
N-hexadecanoyl-γGlu-OEG-Ala-Ala-Pro-Phe-OH
N-hexadecanoyl-Glu-Ala-Ala-Pro-Phe-OH
N-tetradecanoyl-bAla-bAla-Pro-Phe-OH
N-tetradecanoyl-bAla-bAla-bAla-Pro-Phe-OH
N-tetradecanoyl-Ala-Ala-Ala-Ala-Pro-Phe-OH
N-dodecanoyl-Ala-Ala-Ala-Ala-Ala-Pro-Phe-OH
N-myristoyl-Leu-Ala-Ala-Pro-Tyr-OH
N-myristoyl-Glu-Ala-Ala-Pro-Trp-OH
N-palmitoyl-Glu-Ala-Ala-Pro-DPhe-OH
N-myristoyl-Leu-bAla-Ala-Pro-DPhe-OH
N-hexadecanoyl-γGlu-Ala-Pro-Phe-OH
N-octadecanoyl-γGlu-Ala-Pro-Phe-OH
N-eicosanoyl-γGlu-Ala-Pro-Phe-OH
N-tetradecanoyl-Trp-Pro-Tyr-OH
N-dodecanoyl-Leu-Thr-Trp-Pro-Tyr-OH
N-hexadecanoyl-γGlu-DAla-DPro-DPhe-OH
N-tetradecanoyl-γGlu-DAla-DAla-DPro-DPhe-OH
N-tetradecanoyl-Leu-Ala-Ala-Pro-Phe-OH
N-octadecanoyl-γGlu-Ala-Ala-Pro-Phe-OH
N-eicosanoyl-γGlu-Ala-Ala-Pro-Phe-OH
N-tetradecanoyl-γGlu-Ala-Ala-Pro-Phe-OH
N-tetradecanoyl-His-Ala-Ala-Pro-Phe-OH
N-tetradecanoyl-Thr-Ala-Ala-Pro-Phe-OH
N-hexadecanoyl-Thr-Ala-Ala-Pro-Phe-OH
N-tetradecanoyl-γGlu-Ala-Pro-Trp-OH
N-tetradecanoyl-His-Ala-Arg-Pro-Phe-OH
N-tetradecanoyl-DAla-DAla-DPro-DPhe-OH
N-tetradecanoyl-L-Ala-L-Ala-L-Pro-D-Phe-OH
N-hexadecanoyl-Glu-Ala-Ala-Pro-D-Phe-OH
N-tetradecanoyl-Glu-Ala-Ala-Pro-Trp-OH
N-tetradecanoyl-His-Ala-Trp-Pro-Phe-OH
N-tetradecanoyl-γGlu-His-Ala-Arg-Pro-Phe-OH
N-tetradecanoyl-DHis-DAla-DArg-DPro-DPhe-OH
N-tetradecanoyl-εLys-His-Ala-Arg-Pro-Phe-OH
N-tetradecanoyl-Arg-His-Ala-Arg-Pro-Phe-OH

In one aspect, an N-terminally fatty acid modified peptides or oligopeptides of the invention is selected from the group consisting of:
N-eicosanoyl-γGlu-Ala-Ala-Pro-Phe-OH
N-eicosanoyl-γGlu-Ala-Pro-Phe-OH
N-hexadecanoyl-Ala-Ala-Pro-Phe-OH
N-hexadecanoyl-γGlu-Ala-Ala-Pro-Phe-OH
N-hexadecanoyl-γGlu-Ala-Pro-Phe-OH
N-hexadecanoyl-γGlu-DAla-DPro-DPhe-OH
N-hexadecanoyl-γGlu-OEG-Ala-Ala-Pro-Phe-OH
N-hexadecanoyl-Glu-Ala-Ala-Pro-Phe-OH
N-hexadecanoyl-Thr-Ala-Ala-Pro-Phe-OH
N-Myristoyl-Leu-Ala-Ala-Pro-Tyr-OH
N-octadecanoyl-γGlu-Ala-Ala-Pro-Phe-OH
N-octadecanoyl-γGlu-Ala-Pro-Phe-OH
N-Palmitoyl-Glu-Ala-Ala-Pro-DPhe-OH
N-tetradecanoyl-Ala-Ala-Pro-DPhe-OH
N-tetradecanoyl-Ala-Ala-Pro-Phe-OH
N-tetradecanoyl-DAla-DAla-DPro-DPhe-OH
N-tetradecanoyl-γGlu-Ala-Pro-Phe-OH
N-tetradecanoyl-γGlu-DAla-DAla-DPro-DPhe-OH
N-tetradecanoyl-γGlu-DAla-DPro-DPhe-OH
N-tetradecanoyl-His-Ala-Arg-Pro-Phe-OH
N-tetradecanoyl-His-Ala-Trp-Pro-Phe-OH
N-tetradecanoyl-Leu-Ala-Ala-Pro-Phe-OH
N-tetradecanoyl-βAla-Ala-Pro-Phe-OH
N-tetradecanoyl-Thr-Ala-Ala-Pro-Phe-OH

In one aspect, an N-terminally fatty acid modified peptides or oligopeptides of the invention is selected from the group consisting of:
N-dodecanoyl-Ala-Ala-Pro-Phe-OH
N-dodecanoyl-Ala-Ala-Pro-Trp-OH
N-dodecanoyl-DAla-DAla-DPro-DPhe-OH
N-dodecanoyl-Leu-Thr-Trp-Pro-Tyr-OH
N-eicosanoyl-γGlu-Ala-Pro-Phe-OH
N-hexadecanoyl-Ala-Ala-Pro-Phe-OH
N-hexadecanoyl-γGlu-Ala-Ala-Pro-Phe-OH
N-hexadecanoyl-γGlu-Ala-Pro-Phe-OH
N-hexadecanoyl-γGlu-DAla-DPro-DPhe-OH
N-hexadecanoyl-Thr-Ala-Ala-Pro-Phe-OH
N-Myristoyl-Glu-Ala-Ala-Pro-Trp-OH
N-Myristoyl-Leu-Ala-Ala-Pro-Tyr-OH
N-Myristoyl-Leu-bAla-Ala-Pro-DPhe-OH
N-octadecanoyl-γGlu-Ala-Pro-Phe-OH
N-Palmitoyl-Glu-Ala-Ala-Pro-DPhe-Ona
N-tetradecanoy-γGlu-OEG-Ala-Ala-Pro-Phe-OH
N-tetradecanoyl-Ala-Ala-Pro-DPhe-OH
N-tetradecanoyl-Ala-Ala-Pro-Phe-OH
N-tetradecanoyl-bAla-bAla-Pro-Phe-OH
N-tetradecanoyl-DAla-DAla-DPro-DPhe-OH
N-tetradecanoyl-γGlu-Ala-Pro-Phe-OH
N-tetradecanoyl-γGlu-DAla-DPro-DPhe-OH
N-tetradecanoyl-Glu-Ala-Ala-Pro-Trp-OH
N-tetradecanoyl-His-Ala-Arg-Pro-Phe-OH
N-tetradecanoyl-Leu-Ala-Ala-Pro-Phe-OH

In one aspect, an N-terminally fatty acid modified peptides or oligopeptides of the invention is selected from the group consisting of:
N-dodecanoyl-Ala-Ala-Ala-Ala-Ala-Pro-Phe-OH
N-dodecanoyl-Ala-Ala-Pro-Trp-OH
N-dodecanoyl-DAla-DAla-DPro-DPhe-OH
N-dodecanoyl-γGlu-Ala-Pro-Phe-OH
N-dodecanoyl-Leu-Thr-Trp-Pro-Tyr-OH
N-dodecanoyl-OEG-OEG-DPhe-OH
N-eicosanoyl-γGlu-Ala-Ala-Pro-Phe-OH
N-hexadecanoyl-Ala-Ala-Pro-Phe-OH
N-hexadecanoyl-γGlu-Ala-Ala-Pro-Phe-OH
N-hexadecanoyl-Glu-Ala-Ala-Pro-D-Phe-OH
N-hexadecanoyl-Thr-Ala-Ala-Pro-Phe-OH
N-Myristoyl-Leu-Ala-Ala-Pro-Tyr-OH
N-Palmitoyl-Glu-Ala-Ala-Pro-DPhe-OH
N-tetradecanoyl-Ala-Ala-Ala-Ala-Pro-Phe-OH
N-tetradecanoyl-Ala-Ala-Pro-DPhe-OH
N-tetradecanoyl-Ala-Ala-Pro-Phe-OH
N-tetradecanoyl-DAla-DAla-DPro-DPhe-OH
N-tetradecanoyl-γGlu-Ala-Pro-Phe-OH
N-tetradecanoyl-Glu-Ala-Ala-Pro-Trp-OH
N-tetradecanoyl-His-Ala-Arg-Pro-Phe-OH
N-tetradecanoyl-Leu-Ala-Ala-Pro-Phe-OH
N-tetradecanoyl-Thr-Ala-Ala-Pro-Phe-OH
N-tetradecanoyl-Trp-Pro-Tyr-OH

The production of polypeptides is well known in the art. Polypeptides, such as the peptide part of an N-terminally fatty acid modified peptide or oligopeptide of the invention, may for instance be produced by classical peptide synthesis, e.g. solid phase peptide synthesis using t-Boc or Fmoc chemistry or other well established techniques, see e.g. Greene and Wuts, “Protective Groups in Organic Synthesis”, John Wiley & Sons, 1999. The polypeptides may also be produced by a method which comprises culturing a host cell containing a DNA sequence encoding the polypeptide and capable of expressing the polypeptide in a suitable nutrient medium under conditions permitting the expression of the peptide. For polypeptides comprising non-natural amino acid residues, the recombinant cell should be modified such that the non-natural amino acids are incorporated into the polypeptide, for instance by use of tRNA mutants.

The nomenclature for the N-terminally acylated peptide or oligopeptides of the invention used throughout this application is as follows:

N-dodecanoyl-DAla-DAla-DPro-DPhe-OH refers to the structure below where the N-terminus of the tetrapeptide D-alanyl-D-alanyl-D-prolyl-D-phenylalanine is acylated with dodecanoic acid. Alternative name of this structure is (R)-2-({(R)-1-[(R)-2-((R)-2-Dodecanoylamino-propionylamino)-propionyl]-pyrrolidine-2-carbonyl}-amino)-3-phenyl-propionic acid. If the stereochemistry of the amino acid is not specified, it is understood to be the naturally occurring L-amino acid. γGlu refers to gamma-L-glutamyl; βAla refers to beta-L-alanyl etcetera.

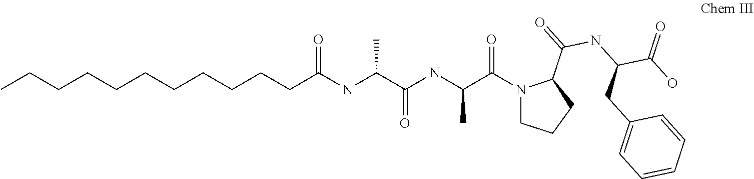 [SMILES: CCCCCCCCCCCC(=O)N[C@H](C)C(=O)N[C@H](C)C(=O)N1CCC[C@@H]1C(=O)N[C@H](Cc1ccccc1)C(=O)O]

Active Ingredient

The term “active ingredient” is herein used for any drug substance in a pharmaceutical drug that is biologically active, i.e. a small molecule, a peptide or a protein that provides pharmacological activity or other direct effect in the cure, treatment, or prevention of disease, or to affect the structure or any function of the body of man or animals. Alternative terms include active pharmaceutical ingredient (API) and bulk active.

The term “pharmaceutically active peptide or protein” is herein used for any active ingredient in a pharmaceutical drug which is in the form of a peptide or protein, i.e. a peptide or protein that is biologically active and thus provides pharmacological activity or other direct effect in the cure, treatment, or prevention of disease, or to affect the structure or any function of the body of man or animals.

In one aspect of the invention, the active ingredient is a peptide or protein.

In one aspect of the invention, the active ingredient is selected from an insulin peptide and a GLP-1 peptide.

In one aspect of the invention, the active ingredient is a GLP-1 peptide.

The term “GLP-1 peptide” as used herein means a peptide which is either human GLP-1 or an analog or a derivative thereof with GLP-1 activity.

The term “human GLP-1” or “native GLP-1” as used herein means the human GLP-1 hormone whose structure and properties are well-known. Human GLP-1 is also denoted GLP-1(7-37), it has 31 amino acids and is the result from selective cleavage of the proglucagon molecule.

The GLP-1 peptides of the invention have GLP-1 activity. This term refers to the ability to bind to the GLP-1 receptor and initiate a signal transduction pathway resulting in insulinotropic action or other physiological effects as is known in the art. For example, the analogues and derivatives of the invention can be tested for GLP-1 activity using a standard GLP-1 activity assay.

The term “GLP-1 analogue” as used herein means a modified human GLP-1 wherein one or more amino acid residues of human GLP-1 have been substituted by other amino acid residues and/or wherein one or more amino acid residues have been deleted from human GLP-1 and/or wherein one or more amino acid residues have been added and/or inserted to human GLP-1.

In one aspect a GLP-1 analogue comprises 10 amino acid modifications (substitutions, deletions, additions (including insertions) and any combination thereof) or less relative to human GLP-1, alternatively 9, 8, 7, 6, 5, 4, 3 or 2 modifications or less, yet alternatively 1 modification relative to human GLP-1.

Modifications in the GLP-1 molecule are denoted stating the position, and the one or three letter code for the amino acid residue substituting the native amino acid residue.

When using sequence listing, the first amino acid residue of a sequence is assigned no. 1. However, in what follows—according to established practice in the art for GLP-1 peptides—this first residue is referred to as no. 7, and subsequent amino acid residues are numbered accordingly, ending with no. 37. Therefore, generally, any reference herein to an amino acid residue number or a position number of the GLP-1(7-37) sequence is to the sequence starting with His at position 7 and ending with Gly at position 37. Using the one letter codes for amino acids, terms like 34E, 34Q, or 34R designates that the amino acid in the position 34 is E, Q and R, respectively. Using the three letter codes for amino acids, the corresponding expressions are 34Glu, 34Gln and 34Arg, respectively.

By “des7” or “(or Des)” is meant a native GLP-1 lacking the N-terminal amino acid, histidine. Thus, e.g., des7GLP-1(7-37) is an analogue of human GLP-1 where the amino acid in position 7 is deleted. This analogue may also be designated GLP-1(8-37). Similarly, (des7+des8); (des7, des8); (des7-8); or (Des⁷, Des⁸) in relation to an analogue of GLP-1(7-37), where the reference to GLP-1(7-37) may be implied, refers to an analogue in which the amino acids corresponding to the two N-terminal amino acids of native GLP-1, histidine and alanine, have been deleted. This analogue may also be designated GLP-1(9-37).

Examples of GLP-1 analogues are such wherein glycine in position 37 of GLP-1(7-37) is substituted with lysine to result in K³⁷-GLP-1(7-37). Another non-limiting example of an analogue of the invention is [Aib⁸,Arg³⁴]GLP-1(7-37), which designates a GLP-1(7-37) analogue, in which the alanine at position 8 has been substituted with α-aminoisobutyric acid (Aib) and the lysine at position 34 has been substituted with arginine. This analogue may also be designated (8Aib, R34) GLP-1(7-37). Yet another non-limiting example of an analogue of the invention is [Aib⁸,Arg³⁴,Lys³⁷]GLP-1(7-37), which designates a GLP-1(7-37) analogue, in which the alanine at position 8 has been substituted with α-aminoisobutyric acid (Aib), the lysine at position 34 has been substituted with arginine, and the glycine at position 37 has been substituted with lysine. This analogue may also be designated (8Aib, R34, K37) GLP-1(7-37). A still further non-limiting example of an analogue of the invention is an analogue comprising Imp⁷, and/or (Aib⁸ or S⁸), which refers to a GLP-1(7-37) analogue, which, when compared to native GLP-1, comprises a substitution of histidine at position 7 with imidazopropionic acid (Imp); and/or a substitution of alanine at position 8 with α-aminoisobutyric acid (Aib), or with serine.]

Further examples of GLP-1 analogues include:.[Aib⁸,Arg³⁴]GLP-1(7-37), Arg³⁴GLP-1(7-37), [Aib⁸,Arg³⁴,Lys³⁷]GLP-1(7-37).

The term “GLP-1 derivative” as used herein means a chemically modified parent GLP-1(7-37) or an analogue thereof, wherein the modification(s) are in the form of attachment of amides, carbohydrates, alkyl groups, acyl groups, esters, PEGylations, combinations thereof, and the like.

In one aspect of the invention, the modification(s) include attachment of a side chain to GLP-1(7-37) or an analogue thereof. In a particular aspect, the side chain is capable of forming non-covalent aggregates with albumin, thereby promoting the circulation of the derivative with the blood stream, and also having the effect of protracting the time of action of the derivative, due to the fact that the aggregate of the GLP-1-derivative and albumin is only slowly disintegrated to release the active ingredient. Thus, the substituent, or side chain, as a whole is preferably referred to as an albumin binding moiety. In particular aspects, the side chain has at least 10 carbon atoms, or at least 12, 14, 16, 18, 20, 22, or at least 24 carbon atoms. In further particular aspects, the side chain may further include at least 5 hetero atoms, in particular O and N, for example at least 7, 9, 10, 12, 15, 17, or at least 20 hetero atoms, such as at least 1, 2, or 3 N-atoms, and/or at least 3, 6, 9, 12, or 15 O-atoms.

In another particular aspect the albumin binding moiety comprises a portion which is particularly relevant for the albumin binding and thereby the protraction, which portion may accordingly be referred to as a “protracting moiety”. The protracting moiety may be at, or near, the opposite end of the albumin binding moiety, relative to its point of attachment to the peptide.

In a still further particular aspect the albumin binding moiety comprises a portion in between the protracting moiety and the point of attachment to the peptide, which portion may be referred to as a “linker”, “linker moiety”, “spacer”, or the like. The linker may be optional, and hence in that case the albumin binding moiety may be identical to the protracting moiety.

In particular aspects, the albumin binding moiety and/or the protracting moiety is lipophilic, and/or negatively charged at physiological pH (7.4).

The albumin binding moiety, the protracting moiety, or the linker may be covalently attached to a lysine residue of the GLP-1 peptide by acylation. Additional or alternative conjugation chemistry includes alkylation, ester formation, or amide formation, or coupling to a cysteine residue, such as by maleimide or haloacetamide (such as bromo-/fluoro-/iodo-) coupling.

In a preferred aspect, an active ester of the albumin binding moiety, preferably comprising a protracting moiety and a linker, is covalently linked to an amino group of a lysine residue, preferably the epsilon amino group thereof, under formation of an amide bond (this process being referred to as acylation).

Unless otherwise stated, when reference is made to an acylation of a lysine residue, it is understood to be to the epsilon-amino group thereof.

For the present purposes, the terms “albumin binding moiety”, “protracting moiety”, and “linker” may include the unreacted as well as the reacted forms of these molecules. Whether or not one or the other form is meant is clear from the context in which the term is used.

For the attachment to the GLP-1 peptide, the acid group of the fatty acid, or one of the acid groups of the fatty diacid, forms an amide bond with the epsilon amino group of a lysine residue in the GLP-1 peptide, preferably via a linker.

The term “fatty diacid” refers to fatty acids as defined above but with an additional carboxylic acid group in the omega position. Thus, fatty diacids are dicarboxylic acids.

Each of the two linkers of the derivative of the invention may comprise the following first linker element:

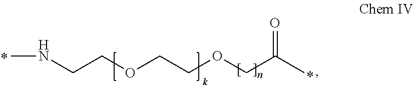

wherein k is an integer in the range of 1-5, and n is an integer in the range of 1-5.

In a particular aspect, when k=1 and n=1, this linker element may be designated OEG, or a di-radical of 8-amino-3,6-dioxaoctanic acid, and/or it may be represented by the following formula:

*—NH—(CH₂)₂—O—(CH₂)₂—O—CH₂—CO—*. Chem V:

In another particular aspect, each linker of the derivative of the invention may further comprise, independently, a second linker element, preferably a Glu di-radical, such as Chem VI and/or Chem VII:

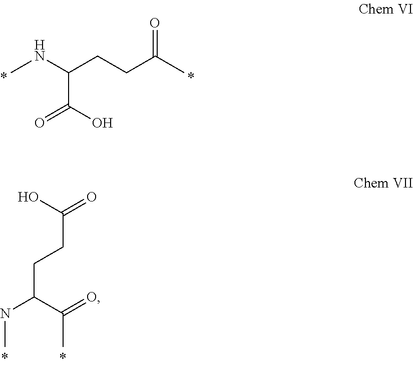

wherein the Glu di-radical may be included p times, where p is an integer in the range of 1-3.

Chem VI may also be referred to as gamma-Glu, or briefly γGlu, due to the fact that it is the gamma carboxy group of the amino acid glutamic acid which is here used for connection to another linker element, or to the epsilon-amino group of lysine. As explained above, the other linker element may, for example, be another Glu residue, or an OEG molecule. The amino group of Glu in turn forms an amide bond with the carboxy group of the protracting moiety, or with the carboxy group of, e.g., an OEG molecule, if present, or with the gamma-carboxy group of, e.g., another Glu, if present.

Chem VII may also be referred to as alpha-Glu, or briefly aGlu, or simply Glu, due to the fact that it is the alpha carboxy group of the amino acid glutamic acid which is here used for connection to another linker element, or to the epsilon-amino group of lysine.

The above structures of Chem. VI and Chem. VII cover the L-form, as well as the D-form of Glu. In particular aspects, Chem. VI and/or Chem. VII is/are, independently, a) in the L-form, or b) in the D-form.

In still further particular aspects the linker has a) from 5 to 41 C-atoms; and/or b) from 4 to 28 hetero atoms.

The concentration in plasma of the GLP-1 derivatives of the invention may be determined using any suitable method. For example, LC-MS (Liquid Chromatography Mass Spectroscopy) may be used, or immunoassays such as RIA (Radio Immuno Assay), ELISA (Enzyme-Linked Immuno Sorbent Assay), and LOCI (Luminescence Oxygen Channeling Immunoasssay). General protocols for suitable RIA and ELISA assays are found in, e.g., WO09/030,738 on p. 116-118.

The conjugation of the GLP-1 analogue and the activated side chain is conducted by use of any conventional method, e.g. as described in the following references (which also describe suitable methods for activation of polymer molecules): R. F. Taylor, (1991), “Protein immobilisation. Fundamental and applications”, Marcel Dekker, N.Y.; S. S. Wong, (1992), “Chemistry of Protein Conjugation and Crosslinking”, CRC Press, Boca Raton; G. T. Hermanson et al., (1993), “Immobilized Affinity Ligand Techniques”, Academic Press, N.Y.). The skilled person will be aware that the activation method and/or conjugation chemistry to be used depends on the attachment group(s) of the polypeptide (examples of which are given further above), as well as the functional groups of the polymer (e.g. being amine, hydroxyl, carboxyl, aldehyde, sulfydryl, succinimidyl, maleimide, vinysulfone or haloacetate).

In one aspect of the invention, the active ingredient is an insulin peptide.

The term “insulin peptide” as used herein means a peptide which is either human insulin or an analog or a derivative thereof with insulin activity.

The term “human insulin” as used herein means the human insulin hormone whose structure and properties are well-known. Human insulin has two polypeptide chains, named the A-chain and the B-chain. The A-chain is a 21 amino acid peptide and the B-chain is a 30 amino acid peptide, the two chains being connected by disulphide bridges: a first bridge between the cysteine in position 7 of the A-chain and the cysteine in position 7 of the B-chain, and a second bridge between the cysteine in position 20 of the A-chain and the cysteine in position 19 of the B-chain. A third bridge is present between the cysteines in position 6 and 11 of the A-chain.

In the human body, the hormone is synthesized as a single-chain precursor proinsulin (preproinsulin) consisting of a prepeptide of 24 amino acids followed by proinsulin containing 86 amino acids in the configuration: prepeptide-B-Arg Arg-C-Lys Arg-A, in which C is a connecting peptide of 31 amino acids. Arg-Arg and Lys-Arg are cleavage sites for cleavage of the connecting peptide from the A and B chains.

An insulin peptide according to the invention has at least 2% Insulin Receptor affinity as defined below.

The term “insulin analogue” as used herein means a modified human insulin wherein one or more amino acid residues of the insulin have been substituted by other amino acid residues and/or wherein one or more amino acid residues have been deleted from the insulin and/or wherein one or more amino acid residues have been added and/or inserted to the insulin.

In one aspect an insulin analogue comprises 10 amino acid modifications (substitutions, deletions, additions (including insertions) and any combination thereof) or less relative to human insulin, alternatively 9, 8, 7, 6, 5, 4, 3 or 2 modifications or less, yet alternatively 1 modification relative to human insulin.

Modifications in the insulin molecule are denoted stating the chain (A or B), the position, and the one or three letter code for the amino acid residue substituting the native amino acid residue.

By “connecting peptide” or “C-peptide” is meant a connection moiety “C” of the B-C-A polypeptide sequence of a single chain proinsulin-molecule. In the human insulin chain, the C-peptide connects position 30 of the B chain and position 1 of the A chain and is 35 amino acid residue long. The connecting peptide includes two terminal dibasic amino acid sequence, e.g., Arg-Arg and Lys-Arg which serve as cleavage sites for cleavage off of the connecting peptide from the A and B chains to form the two-chain insulin molecule.

By “desB30” or “B(1-29)” is meant a natural insulin B chain or an analogue thereof lacking the B30 amino acid and “A(1-21)” means the natural insulin A chain. Thus, e.g., A14Glu,B25His,desB30 human insulin is an analogue of human insulin where the amino acid in position 14 in the A chain is substituted with glutamic acid, the amino acid in position 25 in the B chain is substituted with histidine, and the amino acid in position 30 in the B chain is deleted.

Herein terms like “A1”, “A2” and “A3” etc. indicates the amino acid in position 1, 2 and 3 etc., respectively, in the A chain of insulin (counted from the N-terminal end). Similarly, terms like B1, B2 and B3 etc. indicates the amino acid in position 1, 2 and 3 etc., respectively, in the B chain of insulin (counted from the N-terminal end). Using the one letter codes for amino acids, terms like A21A, A21G and A21Q designates that the amino acid in the A21 position is A, G and Q, respectively. Using the three letter codes for amino acids, the corresponding expressions are A21Ala, A21Gly and A21Gln, respectively.

Examples of insulin analogues are such wherein the amino acid in position A14 is Asn, Gln, Glu, Arg, Asp, Gly or His, the amino acid in position B25 is His and which optionally further comprises one or more additional mutations. Furthermore, the amino acid in position B16 may be substituted with Glu or His. Further examples of insulin analogues are the deletion analogues, e.g., analogues where the B30 amino acid in human insulin has been deleted (des(B30) human insulin), insulin analogues wherein the B1 amino acid in human insulin has been deleted (des(B1) human insulin), des(B28-B30) human insulin and desB27 human insulin. Insulin analogues wherein the A-chain and/or the B-chain have an N-terminal extension and insulin analogues wherein the A-chain and/or the B-chain have a C-terminal extension such as with two arginine residues added to the C-terminal of the B-chain are also examples of insulin analogues. Further examples are insulin analogues comprising combinations of the mentioned mutations.

Further examples of insulin analogues include: DesB30 human insulin;

GluA14,HisB25 human insulin; HisA14,HisB25 human insulin; GluA14,HisB25,desB30 human insulin; HisA14, HisB25,desB30 human insulin;
GluA14,HisB25,desB27,desB28,desB29,desB30 human insulin;
GluA14,HisB25,GluB27,desB30 human insulin; GluA14,HisB16,HisB25,desB30 human insulin; HisA14,HisB16,HisB25,desB30 human insulin;
HisA8,GluA14,HisB25,GluB27,desB30 human insulin;
HisA8,GluA14,GluB1,GluB16,HisB25,GluB27,desB30 human insulin;
HisA8,GluA14,GluB16,HisB25,desB30 human insulin; GluA14, desB27, desB30 human insulin; CysA10, GluA14, CysB3, HisB25, desB30 human insulin; CysA10, GluA14, CysB3,
HisB25, desB27, desB30 human insulin; CysA10, GluA14, CysB4, HisB25, desB30 human insulin; CysA10, GluA14, CysB4, HisB25, desB27, desB30; human insulin; CysA10, GluA14, CysB4, desB27, desB30 human insulin; human insulin; and CysA10, GluA14, CysB3, desB27, desB30 human insulin.

The term “insulin derivative” as used herein means a chemically modified parent insulin or an analogue thereof, wherein the modification(s) are in the form of attachment of amides, carbohydrates, alkyl groups, acyl groups, esters, PEGylations, and the like.

In one aspect of the invention, the modification(s) include attachment of a side chain to human insulin or an analogue thereof. In a particular aspect, the side chain is capable of forming non-covalent aggregates with albumin, thereby promoting the circulation of the derivative with the blood stream, and also having the effect of protracting the time of action of the derivative, due to the fact that the aggregate of the insulin-derivative and albumin is only slowly disintegrated to release the active ingredient. Thus, the substituent, or side chain, as a whole is preferably referred to as an albumin binding moiety. In particular aspects, the side chain has at least 10 carbon atoms, or at least 12, 14, 16, 18, 20, 22, or at least 24 carbon atoms. In further particular aspects, the side chain may further include at least 5 hetero atoms, in particular O and N, for example at least 7, 9, 10, 12, 15, 17, or at least 20 hetero atoms, such as at least 1, 2, or 3 N-atoms, and/or at least 3, 6, 9, 12, or 15 O-atoms.

In another particular aspect the albumin binding moiety comprises a portion which is particularly relevant for the albumin binding and thereby the protraction, which portion may accordingly be referred to as a “protracting moiety”. The protracting moiety may be at, or near, the opposite end of the albumin binding moiety, relative to its point of attachment to the peptide.

In a still further particular aspect the albumin binding moiety comprises a portion in between the protracting moiety and the point of attachment to the peptide, which portion may be referred to as a “linker”, “linker moiety”, “spacer”, or the like. The linker may be optional, and hence in that case the albumin binding moiety may be identical to the protracting moiety.

In particular aspects, the albumin binding moiety and/or the protracting moiety is lipophilic, and/or negatively charged at physiological pH (7.4).

The albumin binding moiety, the protracting moiety, or the linker may be covalently attached to a lysine residue of human insulin or an insulin analogue by acylation. Additional or alternative conjugation chemistry includes alkylation, ester formation, or amide formation, or coupling to a cysteine residue, such as by maleimide or haloacetamide (such as bromo-/fluoro-/iodo-) coupling.

In one aspect, an active ester of the albumin binding moiety, preferably comprising a protracting moiety and a linker, is covalently linked to an amino group of a lysine residue, preferably the epsilon amino group thereof, under formation of an amide bond (this process being referred to as acylation).

Unless otherwise stated, when reference is made to an acylation of a lysine residue, it is understood to be to the epsilon-amino group thereof.

For the present purposes, the terms “albumin binding moiety”, “protracting moiety”, and “linker” may include the unreacted as well as the reacted forms of these molecules. Whether or not one or the other form is meant is clear from the context in which the term is used.

For the attachment to human insulin or the insulin analogue, the acid group of the fatty acid, or one of the acid groups of the fatty diacid, forms an amide bond with the epsilon amino group of a lysine residue in human insulin or the insulin analogue, preferably via a linker.

The term “fatty diacid” refers to fatty acids as defined above but with an additional carboxylic acid group in the omega position. Thus, fatty diacids are dicarboxylic acids.

Each of the two linkers of the derivative of the invention may comprise the following first linker element:

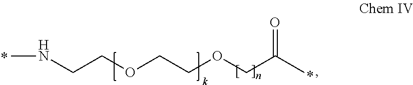

wherein k is an integer in the range of 1-5, and n is an integer in the range of 1-5.

In a particular aspect, when k=1 and n=1, this linker element may be designated OEG, or a di-radical of 8-amino-3,6-dioxaoctanic acid, and/or it may be represented by the following formula:

*—NH—(CH₂)₂—O—(CH₂)₂—O—CH₂—CO—*. Chem V:

In another particular aspect, each linker of the derivative of the invention may further comprise, independently, a second linker element, preferably a Glu di-radical, such as Chem VI and/or Chem VII:

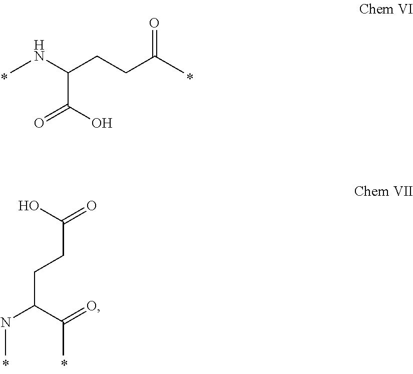

wherein the Glu di-radical may be included p times, where p is an integer in the range of 1-3.

Chem VI may also be referred to as gamma-Glu, or briefly γGlu, due to the fact that it is the gamma carboxy group of the amino acid glutamic acid which is here used for connection to another linker element, or to the epsilon-amino group of lysine. As explained above, the other linker element may, for example, be another Glu residue, or an OEG molecule. The amino group of Glu in turn forms an amide bond with the carboxy group of the protracting moiety, or with the carboxy group of, e.g., an OEG molecule, if present, or with the gamma-carboxy group of, e.g., another Glu, if present.

Chem VII may also be referred to as alpha-Glu, or briefly aGlu, or simply Glu, due to the fact that it is the alpha carboxy group of the amino acid glutamic acid which is here used for connection to another linker element, or to the epsilon-amino group of lysine.

The above structures of Chem VI and Chem VII cover the L-form, as well as the D-form of Glu. In particular aspects, Chem VI and/or Chem VII is/are, independently, a) in the L-form, or b) in the D-form.

In still further particular aspects the linker has a) from 5 to 41 C-atoms; and/or b) from 4 to 28 hetero atoms.

Non-limiting examples of derivatives of human insulin and deriviatives of insulin analogues for use in pharmaceutical compositions comprising an N-terminally modified peptide or oligopeptide according to the invention include human insulin B30 threonine methyl ester, GlyA21,ArgB31,Arg-amideB32 human insulin, N^{εB29}-tetradecanoyl desB30 human insulin, N^{εB29}-tetradecanoyl human insulin, N^{εB29}-decanoyl desB30 human insulin, N^{εB29}-dodecanoyl desB30 human insulin, N^{εB29}-3-(2-{2-(2-methoxy-ethoxy)-ethoxy}-ethoxy)-propionyl human insulin, LysB29(Nε-hexadecandioyl-γGlu) des(B30) human insulin), N^{εB29}-(Nα-(Sar-OC(CH2)13CO)-γGlu) desB30 human insulin, N^{εB29}-ω-carboxy-pentadecanoyl-γ-L-glutamylamide desB30 human insulin, N^{δ}B29-hexadecandioyl-γ-amino-butanoyl desB30 human insulin, N^{ε}B29-hexadecandioyl-γ-L-Glu-amide desB30 insulin, A14E, B25H, B29K(N(eps)Octadecanedioyl-γGlu-OEG-OEG), desB30 human insulin, A14E, B16H, B25H, B29K((N(eps)Eicosanedioyl-γGlu-[2-(2-{2-[2-(2-aminoethoxy)ethoxy]acetylamino}ethoxy)-ethoxy]acetyl)), desB30 human insulin and A14E, B25H, desB27, B29K(N-(eps)-(octadecandioyl-γGlu), desB30 human insulin.

The term “PEGylated insulin” means an insulin analogue having a PEG molecule conjugated to one or more amino acids.

The term “polyethylene glycol” or “PEG” means a polyethylene glycol compound or a derivative thereof.

To effect covalent attachment of the polymer molecule(s) to the insulin analogue, the hydroxyl end groups of the polymer molecule are provided in activated form, i.e. with reactive functional groups. Suitable activated polymer molecules are commercially available, e.g. from Shearwater Corp., Huntsville, Ala., USA, or from PolyMASC Pharmaceuticals plc, UK. Alternatively, the polymer molecules can be activated by conventional methods known in the art, e.g. as disclosed in WO 90/13540. Specific examples of activated linear or branched polymer molecules for use in the present invention are described in the Shearwater Corp. 1997 and 2000 Catalogs (Functionalized Biocompatible Polymers for Research and pharmaceuticals, Polyethylene Glycol and Derivatives, incorporated herein by reference). Specific examples of activated PEG polymers include the following linear PEGs: NHS-PEG (e.g. SPA-PEG, SSPA-PEG, SBA-PEG, SS-PEG, SSA-PEG, SC-PEG, SG-PEG, and SCM-PEG), and NOR-PEG), BTC-PEG, EPDX-PEG, NCO-PEG, NPC-PEG, CDI-PEG, ALD-PEG, TRES-PEG, VS-PEG, IODO-PEG, and MAL-PEG, and branched PEGs such as PEG2-NHS and those disclosed in U.S. Pat. No. 5,932,462 and U.S. Pat. No. 5,643,575.

The conjugation of the polypeptide and the activated polymer molecules is conducted by use of any conventional method, e.g. as described in the following references (which also describe suitable methods for activation of polymer molecules): R. F. Taylor, (1991), “Protein immobilisation. Fundamental and applications”, Marcel Dekker, N.Y.; S. S. Wong, (1992), “Chemistry of Protein Conjugation and Crosslinking”, CRC Press, Boca Raton; G. T. Hermanson et al., (1993), “Immobilized Affinity Ligand Techniques”, Academic Press, N.Y.). The skilled person will be aware that the activation method and/or conjugation chemistry to be used depends on the attachment group(s) of the polypeptide (examples of which are given further above), as well as the functional groups of the polymer (e.g. being amine, hydroxyl, carboxyl, aldehyde, sulfydryl, succinimidyl, maleimide, vinysulfone or haloacetate).

Oral Pharmaceutical Compositions

Oral pharmaceutical compositions, alternatively termed oral pharmaceutical formulations, oral compositions or oral formulations, comprising N-terminally fatty acid modified peptides or oligopeptides as herein described are also contemplated by the invention. In one aspect an oral pharmaceutical composition is a composition comprising an active ingredient and an N-terminally fatty acid modified peptide or oligopeptide of the invention. In one aspect an oral pharmaceutical composition is a composition comprising an active ingredient, an N-terminally fatty acid modified peptide or oligopeptide of the invention and additional excipient(s).

In one aspect an oral pharmaceutical composition is a composition comprising an active ingredient, one or more lipids and an N-terminally fatty acid modified peptide or oligopeptide of the invention.

In one aspect, an oral pharmaceutical composition comprising an active ingredient and an N-terminally fatty acid modified peptide or oligopeptide of the invention is in the form of a solid dosage form. In one aspect, an oral pharmaceutical composition comprising an active ingredient and an N-terminally fatty acid modified peptide or oligopeptide of the invention is in the form of a tablet. In one aspect, an oral pharmaceutical composition comprising an active ingredient and an N-terminally fatty acid modified peptide or oligopeptide of the invention is delivered in a capsule.

The term “excipient” as used herein broadly refers to any component other than the active ingredient and the N-terminally fatty acid modified peptide or oligopeptide of the invention. The excipient may be an inert substance, which is inert in the sense that it substantially does not have any therapeutic and/or prophylactic effect per se. In one aspect, the additional excipient(s) of an oral pharmaceutical composition comprising an N-terminally fatty acid modified peptide or oligopeptide of the invention includes diluent(s), binder(s), granulating agent(s), glidant(s) (i.e. flow aid(s)), lubricant(s) to ensure efficient tabletting, disintegrant(s) to promote tablet break-up in the digestive tract; sweetener(s), flavour(s), and/or pigment(s). A person skilled in the art may select one or more of the aforementioned excipients with respect to the particular desired properties of the solid oral dosage form by routine experimentation and without any undue burden. The amount of each excipient used may vary within ranges conventional in the art. Techniques and excipients which may be used to formulate oral pharmaceutical compositions are described in Handbook of Pharmaceutical Excipients, 6th edition, Rowe et al., Eds., American Pharmaceuticals Association and the Pharmaceutical Press, publications department of the Royal Pharmaceutical Society of Great Britain (2009); and Remington: the Science and Practice of Pharmacy, 21th edition, Gennaro, Ed., Lippincott Williams & Wilkins (2005).

In one aspect of the invention, a polymer coating is applied to the oral pharmaceutical composition.

In one aspect of the invention, the oral pharmaceutical composition is in the form of a tablet and the weight of the tablet is in the range of from 150 mg to 1000 mg, such as in the range of 300-600 mg or such as 300-500 mg.

In one aspect of the invention, the active ingredient is present in the pharmaceutical composition in a concentration between from 0.1 to 30% (w/w) of the total amount of ingredients in the composition. In another aspect the active ingredient is present in a concentration between from 0.5 to 20% (w/w). In another aspect the active ingredient is present in a concentration between from 1 to 10% (w/w).

In one aspect of the invention, the active ingredient is present in the pharmaceutical composition in a concentration between from 0.2 mM to 100 mM. In another aspect the active ingredient is present in a concentration between from 0.5 to 70 mM. In another aspect the active ingredient is present in a concentration between from 0.5 to 35 mM. In another aspect the active ingredient is present in a concentration between from 1 to 30 mM.

The term “lipid” is herein used for a substance, material or ingredient that is more mixable with oil than with water. A lipid is insoluble or almost insoluble in water but is easily soluble in oil or other nonpolar solvents.

A lipid, used for a pharmaceutical composition comprising an active ingredient and an N-terminally fatty acid modified peptide or oligopeptide of the invention, may comprise one or more lipophilic substances, i.e. substances that form homogeneous mixtures with oils and not with water. Multiple lipids may constitute the lipophilic phase of the non-aqueous liquid pharmaceutical composition and form the oil aspect. At room temperature, the lipid can be solid, semisolid or liquid. For example, a solid lipid can exist as a paste, granular form, powder or flake. If more than one excipient comprises the lipid, the lipid can be a mixture of liquids, solids, or both.

Examples of solid lipids i.e., lipids which are solid or semisolid at room temperature, include, but are not limited to, the following:

1. Mixtures of mono-, di- and triglycerides, such as hydrogenated coco-glycerides (melting point (m.p.) of about 33.5° C. to about 37° C.], commercially-available as WITEPSOL HI5 from Sasol Germany (Witten, Germany); Examples of fatty acid triglycerides e.g., C10-C22 fatty acid triglycerides include natural and hydrogenated oils, such as vegetable oils;

2. Esters, such as propylene glycol (PG) stearate, commercially available as MONOSTEOL (m.p. of about 33° C. to about 36° C.) from Gattefosse Corp. (Paramus, N.J.); diethylene glycol palmito stearate, commercially available as HYDRINE (m.p. of about 44.5° C. to about 48.5° C.) from Gattefosse Corp.;

3. Polyglycosylated saturated glycerides, such as hydrogenated palm/palm kernel oil PEG-6 esters (m.p. of about 30.5° C. to about 38° C.), commercially-available as LABRAFIL M2130 CS from Gattefosse Corp. or Gelucire 33/01;

4. Fatty alcohols, such as myristyl alcohol (m.p. of about 39° C.), commercially available as LANETTE 14 from Cognis Corp. (Cincinnati, Ohio); esters of fatty acids with fatty alcohols, e.g., cetyl palmitate (m.p. of about 50° C.); isosorbid monolaurate, e.g. commercially available under the trade name ARLAMOL ISML from Uniqema (New Castle, Del.), e.g. having a melting point of about 43° C.;

5. PEG-fatty alcohol ether, including polyoxyethylene (2) cetyl ether, e.g. commercially available as BRIJ 52 from Uniqema, having a melting point of about 33° C., or polyoxyethylene (2) stearyl ether, e.g. commercially available as BRIJ 72 from Uniqema having a melting point of about 43° C.;

6. Sorbitan esters, e.g. sorbitan fatty acid esters, e.g. sorbitan monopalmitate or sorbitan monostearate, e.g., commercially available as SPAN 40 or SPAN 60 from Uniqema and having melting points of about 43° C. to 48° C. or about 53° C. to 57° C. and 41° C. to 54° C., respectively; and 7. Glyceryl mono-C6-C14-fatty acid esters. These are obtained by esterifying glycerol with vegetable oil followed by molecular distillation. Monoglycerides include, but are not limited to, both symmetric (i.e. β-monoglycerides) as well as asymmetric monoglycerides (α-monoglycerides). They also include both uniform glycerides (in which the fatty acid constituent is composed primarily of a single fatty acid) as well as mixed glycerides (i.e. in which the fatty acid constituent is composed of various fatty acids). The fatty acid constituent may include both saturated and unsaturated fatty acids having a chain length of from e.g. C8-C14. Particularly suitable are glyceryl mono laurate e.g. commercially available as IMWITOR 312 from Sasol North America (Houston, Tex.), (m.p. of about 56° C.-60° C.); glyceryl mono dicocoate, commercially available as IMWITOR 928 from Sasol (m.p. of about 33° C.-37° C.); monoglyceryl citrate, commercially available as IMWITOR 370, (m.p. of about 59 to about 63° C.); or glyceryl mono stearate, e.g., commercially available as IMWITOR 900 from Sasol (rn.p. of about 56° C.-61° C.); or self-emulsifying glycerol mono stearate, e.g., commercially available as IMWITOR 960 from Sasol (m.p. of about 56° C.-61° C.).

Examples of liquid and semisolid lipids, i.e., lipids which are liquid or semisolid at room temperature include, but are not limited to, the following:

1. Mixtures of mono-, di- and triglycerides, such as medium chain mono- and diglycerides, glyceryl caprylate/caprate, commercially-available as CAPMUL MCM from Abitec Corp. (Columbus, Ohio); and glycerol monocaprylate, commercially available as RYLO MG08 Pharma and glycerol monocaprate, commercially available as RYLO MG10 Pharma from DANISCO.

2. Glyceryl mono- or di fatty acid ester, e.g. of C6-C18, e.g. C6-C16 e.g. C8-C10, e.g. C8, fatty acids, or acetylated derivatives thereof, e.g. MYVACET 9-45 or 9-08 from Eastman Chemicals (Kingsport, Tenn.) or IMWITOR 308 or 312 from Sasol;

3. Propylene glycol mono- or di-fatty acid ester, e.g. of C8-C20, e.g. C8-C12, fatty acids, e.g. LAUROGLYCOL 90, SEFSOL 218, or CAPRYOL 90 or CAPMUL PG-8 (same as propylene glycol caprylate) from Abitec Corp. or Gattefosse;

4. Oils, such as safflower oil, sesame oil, almond oil, peanut oil, palm oil, wheat germ oil, corn oil, castor oil, coconut oil, cotton seed oil, soybean oil, olive oil and mineral oil;

5. Fatty acids or alcohols, e.g. C8-C20, saturated or mono- or di-unsaturated, e.g. oleic acid, oleyl alcohol, linoleic acid, capric acid, caprylic acid, caproic acid, tetradecanol, dodecanol, decanol;

6. Medium chain fatty acid triglycerides, e.g. C8-C12, e.g. MIGLYOL 812, or long chain fatty acid triglycerides, e.g. vegetable oils;

7. Transesterified ethoxylated vegetable oils, e.g. commercially available as LABRAFIL M2125 CS from Gattefosse Corp;

8. Esterified compounds of fatty acid and primary alcohol, e.g. C8-C20, fatty acids and C2-C3 alcohols, e.g. ethyl linoleate, e.g. commercially available as NIKKOL VF-E from Nikko Chemicals (Tokyo, Japan), ethyl butyrate, ethyl caprylate oleic acid, ethyl oleate, isopropyl myristate and ethyl caprylate;

9. Essential oils, or any of a class of volatile oils that give plants their characteristic odours, such as spearmint oil, clove oil, lemon oil and peppermint oil;

10. Fractions or constituents of essential oils, such as menthol, carvacrol and thymol;

11. Synthetic oils, such as triacetin, tributyrin;

12. Triethyl citrate, acetyl triethyl citrate, tributyl citrate, acetyl tributyl citrate;

13. Polyglycerol fatty acid esters, e.g. diglyceryl monooleate, e.g. DGMO-C, DGMO-90, DGDO from Nikko Chemicals; and

14. Sorbitan esters, e.g. sorbitan fatty acid esters, e.g. sorbitan monolaurate, e.g. commercially available as SPAN 20 from Uniqema.

15. Phospholipids, e.g. Alkyl-O-Phospholipids, Diacyl Phosphatidic Acids, Diacyl Phosphatidyl Cholines, Diacyl Phosphatidyl Ethanolamines, Diacyl Phosphatidyl Glycerols, Di-O-Alkyl Phosphatidic Acids, L-alpha-Lysophosphatidylcholines (LPC), L-alpha-Lysophosphatidylethanolamines (LPE), L-alpha-Lysophosphatidylglycerol (LPG), L-alpha-Lysophosphatidylinositols (LPI), L-alpha-Phosphatidic acids (PA), L-alpha-Phosphatidylcholines (PC), L-alpha-Phosphatidylethanolamines (PE), L-alpha-Phosphatidylglycerols (PG), Cardiolipin (CL), L-alpha-Phosphatidylinositols (PI), L-alpha-Phosphatidylserines (PS), Lyso-Phosphatidylcholines, Lyso-Phosphatidylglycerols, sn-Glycerophosphorylcholines commercially available from LARODAN, or soybean phospholipid (Lipoid S100) commercially available from Lipoid GmbH.

16. Polyglycerol fatty acid esters, such as polyglycerol oleate (Plurol Oleique from Gattefosse).

In one aspect of the invention, the lipid is one or more selected from the group consisting of mono-, di-, and triglycerides. In a further aspect, the lipid is one or more selected from the group consisting of mono- and diglycerides. In yet a further aspect, the lipid is Capmul MCM or Capmul PG-8. In a still further aspect, the lipid is Capmul PG-8. In a further aspect the lipid is Glycerol monocaprylate (Rylo MG08 Pharma from Danisco).

In one aspect the lipid, used for a pharmaceutical composition comprising an active ingredient and N-terminally fatty acid modified peptide or oligopeptide of the invention, is selected from the group consisting of: Glycerol mono-caprylate (such as e.g. Rylo MG08 Pharma) and Glycerol mono-caprate (such as e.g. Rylo MG10 Pharma from Danisco). In another aspect the lipid is selected from the group consisting of: propyleneglycol caprylate (such as e.g. Capmul PG8 from Abitec or Capryol PGMC, or Capryol 90 from Gattefosse).

In one aspect of the invention, the lipid is present in the pharmaceutical composition in a concentration between from 10% to 90% (w/w) of the total amount of ingredients including the active ingredient in the composition. In another aspect the lipid is present in a concentration between from 10 to 80% (w/w). In another aspect the lipid is present in a concentration between from 10 to 60% (w/w). In another aspect the lipid is present in a concentration between from 15 to 50% (w/w). In another aspect the lipid is present in a concentration between from 15 to 40% (w/w). In another aspect the lipid is present in a concentration between from 20 to 30% (w/w). In another aspect the lipid is present in a concentration of about 25% (w/w).

In one aspect of the invention, the lipid is present in the pharmaceutical composition in a concentration between from 100 mg/g to 900 mg/g of the total amount of ingredients including the active ingredient in the composition. In another aspect the lipid is present in a concentration between from 100 to 800 mg/g. In another aspect the lipid is present in a concentration between from 100 to 600 mg/g. In another aspect the lipid is present in a concentration between from 150 to 500 mg/g. In another aspect the lipid is present in a concentration between from 150 to 400 mg/g. In another aspect the lipid is present in a concentration between from 200 to 300 mg/g. In another aspect the lipid is present in a concentration of about 250 mg/g.

In one aspect of the invention, the cosolvent is present in the pharmaceutical composition in a concentration between from 0% to 30% (w/w) of the total amount of ingredients including the active ingredient in the composition. In another aspect the cosolvent is present in a concentration between from 5% to 30% (w/w). In another aspect the cosolvent is present in a concentration between from 10 to 20% (w/w).

In one aspect of the invention, the cosolvent is present in the pharmaceutical composition in a concentration between from 0 mg/g to 300 mg/g of the total amount of ingredients including the active ingredient in the composition. In another aspect the cosolvent is present in a concentration between from 50 mg/g to 300 mg/g. In another aspect the cosolvent is present in a concentration between from 100 to 200 mg/g.

In one aspect of the invention the oral pharmaceutical composition does not contain oil or any other lipid component or surfactant with an HLB below 7. In a further aspect the composition does not contain oil or any other lipid component or surfactant with an HLB below 8. In a yet further aspect the composition does not contain oil or any other lipid component or surfactant with an HLB below 9. In a yet further aspect the composition does not contain oil or any other lipid component or surfactant with an HLB below 10.

The hydrophilic-lipophilic balance (HLB) of each of the non-ionic surfactants of the liquid non-aqueous pharmaceutical composition of the invention is above 10 whereby high insulin peptide (such as N-terminally modified insulin) drug loading capacity and high oral bioavailability are achieved. In one aspect the non-ionic surfactants according to the invention are non-ionic surfactants with HLB above 11. In one aspect the non-ionic surfactants according to the invention are non-ionic surfactants with HLB above 12.

The term “about” as used herein means in reasonable vicinity of the stated numerical value, such as plus or minus 10%.

A non-limiting example of lipid pharmaceutical compositions, for use as pharmaceutical compositions comprising an N-terminally fatty acid modified peptide or oligopeptide of the invention and an active ingredient, may e.g. be found in the patent applications WO 08/145,728, WO 2010/060667 and WO 2011/086093.

Oral bioavailability and absorption kinetics of the oral pharmaceutical composition comprising an N-terminally fatty acid modified peptide or oligopeptide of the invention may be determined according to Assay (I) as described herein.

Assay (I): Oral Administration to Beagle Dogs

Animals, Dosing and Blood Sampling: Beagle dogs, weighing 6-17 kg during the study period are included in the study. The dogs are dosed in fasting state. The oral pharmaceutical compositions are administered by a single oral dosing to the dogs in groups of 8 dogs. Blood samples are taken at the following time points: predose, 0.25, 0.5, 0.75, 1, 1.5, 2, 2.5, 3, 4, 6, 8, 24, 48, 72, 96, 120, 144, 192 and 240 hours post dosing. The i.v. solution (20 nmol/mL in a pH 7.4 solution comprising 0.1 mg/ml Polysorbate 20 (Tween 20), 5.5 mg/ml Phenol, 1.42 mg/ml Na2HPO4 and 14 mg/ml Propylene Glycol) is dosed in a dose volume of 0.1 mL/kg in the same dog colony in one dosing group (n=8). Blood samples are taken at the following time points: predose, 0.25, 0.5, 0.75, 1, 1.5, 2, 2.5, 3, 4, 6, 8, 24, 48, 72, 96, 120, 144, 192 and 240 hours post dosing.

Preparation of Plasma: All blood samples are collected into test tubes containing Ethylenediaminetetraacetic acid (EDTA) for stabilisation and kept on ice until centrifugation. Plasma is separated from whole blood by centrifugation and the plasma is stored at −20° C. or lower until analysis.

Analysis of Plasma Samples: The plasma is analysed for active ingredient using a Luminescence Oxygen Channeling Immunoassay (LOCI). The LOCI assay employs donor beads coated with streptavidin and acceptor beads conjugated with a monoclonal antibody binding to a mid-molecular region of active ingredient. The other monoclonal antibody, specific for an N-terminal epitope, is biotinylated. In the assay the three reactants are combined with the active ingredient which form a two-sited immuno-complex. Illumination of the complex releases singlet oxygen atoms from the donor beads which channels into the acceptor beads and trigger chemiluminescence which is measured in the EnVision plate reader. The amount of light is proportional to the concentration of active ingredient and the lower limit of quantification (LLOQ) in plasma is 100 μM.

The invention is further described by the following non-limiting embodiments:
1. An N-terminally acylated peptide or oligopeptide having the structure

Cx-Aaa10-Aaa9-Aaa8-Aaa7-Aaa6-Aaa5-Aaa-4-Aaa3-Aaa2-Aaa1-OH; SEQ ID No: 1
Chem I
   where Cx is a fatty acid with a length between 6 and 20 carbon atoms, and
   wherein Aaa1 is an aromatic amino acid; Aaa2 is any amino acid except Lys or Asp; Aaa3 is any amino acid; Aaa-4-10 each is any amino acid or absent.
   2. An N-terminally acylated peptide or oligopeptide according to embodiment 1 wherein Aaa1 is Tyr, Trp or Phe.
   3. An N-terminally acylated peptide or oligopeptide according to embodiment 1 or 2 wherein Aaa1 is Trp.
   4. An N-terminally acylated peptide or oligopeptide according to embodiment 1 or 2 wherein Aaa1 is Phe.
   5. An N-terminally acylated peptide or oligopeptide according to embodiment 1 or 2 wherein Aaa1 is Tyr.
   6. An N-terminally acylated peptide or oligopeptide according to any one of the preceding embodiments wherein Aaa2 is any amino acid except Lys, Asp, Glu and Asn.
   7. An N-terminally acylated peptide or oligopeptide according to any one of the preceding embodiments wherein Aaa2 is Pro, Leu, OEG ([2-(2-aminoethoxy)ethoxy]ethylcarbonyl), γGlu or βAsp.
   8. An N-terminally acylated peptide or oligopeptide according to any one of the preceding embodiments wherein Aaa2 is Pro or Leu.
   9. An N-terminally acylated peptide or oligopeptide according to any one of the preceding embodiments wherein Aaa2 is OEG, γGlu or βAsp.
   10. An N-terminally acylated peptide or oligopeptide according to any one of the preceding embodiments wherein Aaa3 is Arg, Lys, His, Trp, Tyr, Phe, OEG, γGlu or βAsp.
   11. An N-terminally acylated peptide or oligopeptide according to any one of the preceding embodiments wherein Aaa3 is Arg, Lys, His, Trp, Tyr or Phe.
   12. An N-terminally acylated peptide or oligopeptide according to any one of the preceding embodiments wherein Aaa3 is OEG, γGlu or βAsp.
   13. An N-terminally acylated peptide or oligopeptide according to any one of the preceding embodiments wherein Aaa4 is any amino acid.
   14. An N-terminally acylated peptide or oligopeptide according to any one of the preceding embodiments wherein Aaa4 is OEG, γGlu or βAsp.
   15. An N-terminally acylated peptide or oligopeptide according to any one of the preceding embodiments wherein Aaa5 is any amino acid.
   16. An N-terminally acylated peptide or oligopeptide according to any one of the preceding embodiments wherein Aaa6 is any amino acid.
   17. An N-terminally acylated peptide or oligopeptide according to any one of the preceding embodiments wherein Aaa7 is any amino acid.
   18. An N-terminally acylated peptide or oligopeptide according to any one of the preceding embodiments wherein Aaa8 is any amino acid.
   19. An N-terminally acylated peptide or oligopeptide according to any one of the preceding embodiments wherein Aaa9 is any amino acid.
   20. An N-terminally acylated peptide or oligopeptide according to any one of the preceding embodiments wherein Aaa4 is absent.
   21. An N-terminally acylated peptide or oligopeptide according to any one of the preceding embodiments wherein Aaa5 is absent.
   22. An N-terminally acylated peptide or oligopeptide according to any one of the preceding embodiments wherein Aaa6 is absent.
   23. An N-terminally acylated peptide or oligopeptide according to any one of the preceding embodiments wherein Aaa7 is absent.
   24. An N-terminally acylated peptide or oligopeptide according to any one of the preceding embodiments wherein Aaa8 is absent.
   25. An N-terminally acylated peptide or oligopeptide according to any one of the preceding embodiments wherein Aaa9 is absent.
   26. An N-terminally acylated peptide or oligopeptide according to any one of the preceding embodiments wherein Aaa10 is any amino acid except Lys.
   27. An N-terminally acylated peptide or oligopeptide according to any one of the preceding embodiments wherein Aaa10 is Leu, Thr, Lys, Arg, His, OEG, γGlu or βAsp.
   28. An N-terminally acylated peptide or oligopeptide according to any one of the preceding embodiments wherein Aaa10 is Leu, Thr, Lys, Arg or His.
   29. An N-terminally acylated peptide or oligopeptide according to any one of the preceding embodiments wherein Aaa10 is Leu, Lys, Arg or His.
   30. An N-terminally acylated peptide or oligopeptide according to any one of the preceding embodiments wherein Aaa10 is Leu, Thr, Arg or His.
   31. An N-terminally acylated peptide or oligopeptide according to any one of the preceding embodiments wherein Aaa10 is Lys, Arg or His.
   32. An N-terminally acylated peptide or oligopeptide according to any one of the preceding embodiments wherein Aaa10 is any amino acid except a basic amino acid.
   33. An N-terminally acylated peptide or oligopeptide according to any one of the preceding embodiments wherein Aaa10 is a basic amino acid.
   34. An N-terminally acylated peptide or oligopeptide according to any one of the preceding embodiments wherein Aaa10 is OEG, γGlu or βAsp.
   35. An N-terminally acylated peptide or oligopeptide according to any one of the preceding embodiments wherein Aaa8-9 are absent.
   36. An N-terminally acylated peptide or oligopeptide according to any one of the preceding embodiments wherein Aaa7-9 are absent.
   37. An N-terminally acylated peptide or oligopeptide according to any one of the preceding embodiments wherein Aaa6-9 are absent.
   38. An N-terminally acylated peptide or oligopeptide according to any one of the preceding embodiments wherein Aaa5-9 are absent.
   39. An N-terminally acylated peptide or oligopeptide according to any one of the preceding embodiments wherein Aaa-4-9 are absent.
   40. An N-terminally acylated peptide or oligopeptide according to any one of the preceding embodiments wherein Aaa3-9 are absent.
   41. An N-terminally acylated peptide or oligopeptide according to any one of the preceding embodiments wherein the amino acids are L or D amino acids.
   42. An N-terminally acylated peptide or oligopeptide according to any one of the preceding embodiments wherein the amino acids are L amino acids.
   43. An N-terminally acylated peptide or oligopeptide according to any one of the preceding embodiments wherein the amino acids are D amino acids.
   44. An N-terminally acylated peptide or oligopeptide according to any one of the preceding embodiments, wherein the length of the fatty acid is between 8-20 carbon atoms.
   45. An N-terminally acylated peptide or oligopeptide according to any one of the preceding embodiments, wherein the length of the fatty acid is between 10-20 carbon atoms.
   46. An N-terminally acylated peptide or oligopeptide according to any one of the preceding embodiments, wherein the length of the fatty acid is between 10-18 carbon atoms.
   47. An N-terminally acylated peptide or oligopeptide according to any one of the preceding embodiments, wherein the length of the fatty acid is between 10-16 carbon atoms.
   48. An N-terminally acylated peptide or oligopeptide according to any one of the preceding embodiments, wherein the length of the fatty acid is between 10-14 carbon atoms.
   49. An N-terminally acylated peptide or oligopeptide according to any one of the preceding embodiments, wherein the length of the fatty acid is between 12-20 carbon atoms.
   50. An N-terminally acylated peptide or oligopeptide according to any one of the preceding embodiments, wherein the length of the fatty acid is between 12-16 carbon atoms.
   51. An N-terminally acylated peptide or oligopeptide according to any one of the preceding embodiments, wherein the length of the fatty acid is between 12-14 carbon atoms.
   52. An N-terminally acylated peptide or oligopeptide according to any one of the preceding embodiments, wherein the length of the fatty acid is 14-16 carbon atoms.
   53. An N-terminally acylated peptide or oligopeptide according to any one of the preceding embodiments, wherein the length of the fatty acid is 20 carbon atoms.
   54. An N-terminally acylated peptide or oligopeptide according to any one of the preceding embodiments, wherein the length of the fatty acid is 18 carbon atoms.
   55. An N-terminally acylated peptide or oligopeptide according to any one of the preceding embodiments, wherein the length of the fatty acid is 16 carbon atoms.
   56. An N-terminally acylated peptide or oligopeptide according to any one of the preceding embodiments, wherein the length of the fatty acid is 14 carbon atoms.
   57. An N-terminally acylated peptide or oligopeptide according to any one of the preceding embodiments, wherein the length of the fatty acid is 12 carbon atoms.
   58. An N-terminally acylated peptide or oligopeptide according to any one of the preceding embodiments, wherein the length of the fatty acid is 10 carbon atoms.
   59. An N-terminally acylated peptide or oligopeptide according to any one of the preceding embodiments, wherein the length of the fatty acid is 16 carbon atoms and amino acids Aaa4-9 are absent.
   60. An N-terminally acylated peptide or oligopeptide according to any one of the preceding embodiments, wherein the length of the fatty acid is 16 carbon atoms and amino acids Aaa5-9 are absent.
   61. An N-terminally acylated peptide or oligopeptide according to any one of the preceding embodiments, wherein the length of the fatty acid is 16 carbon atoms and amino acids Aaa6-9 are absent.
   62. An N-terminally acylated peptide or oligopeptide according to any one of the preceding embodiments, wherein the length of the fatty acid is 14 carbon atoms and amino acids Aaa4-9 are absent.

63. An N-terminally acylated peptide or oligopeptide according to any one of the preceding embodiments, wherein the length of the fatty acid is 14 carbon atoms and amino acids Aaa5-9 are absent.

64. An N-terminally acylated peptide or oligopeptide according to any one of the preceding embodiments, wherein the length of the fatty acid is 14 carbon atoms and amino acids Aaa6-9 are absent.
65. An N-terminally acylated peptide or oligopeptide according to any one of the preceding embodiments, wherein the length of the fatty acid is 12 carbon atoms and amino acids Aaa4-9 are absent.
66. An N-terminally acylated peptide or oligopeptide according to any one of the preceding embodiments, wherein the length of the fatty acid is 12 carbon atoms and amino acids Aaa5-9 are absent.
67. An N-terminally acylated peptide or oligopeptide according to any one of the preceding embodiments, wherein the length of the fatty acid is 12 carbon atoms and amino acids Aaa6-9 are absent.
68. An N-terminally acylated peptide or oligopeptide according to any one of the preceding embodiments, which is an inhibitor of proteolytic activity in an extract from the gastrointestinal tract (GI tract).
69. An N-terminally acylated peptide or oligopeptide according to any one of the preceding embodiments, which is an inhibitor of proteolytic activity such as proteolytic activity of trypsin, chymotrypsin, elastase, carboxypeptidase and/or aminopeptidase.
70. An N-terminally acylated peptide or oligopeptide according to any one of the preceding embodiments, which is an inhibitor of proteolytic activity of trypsin, chymotrypsin, elastase and/or an extract from the GI tract.
71. An N-terminally acylated peptide or oligopeptide according to any one of the preceding embodiments, which is an inhibitor of proteolytic activity of trypsin, chymotrypsin and/or an extract from the GI tract.
72. An N-terminally acylated peptide or oligopeptide according to any one of the preceding embodiments, which is an inhibitor of chymotrypsin activity.
73. An N-terminally acylated peptide or oligopeptide according to any one of the preceding embodiments, which is an inhibitor of trypsin activity.
74. An N-terminally acylated peptide or oligopeptide according to any one of the preceding embodiments, which is an absorption enhancer useful for oral delivery of an active ingredient which is a peptide or protein.
75. An N-terminally acylated peptide or oligopeptide according to any one of the preceding embodiments, which is an absorption enhancer useful for oral delivery of an insulin peptide or a GLP-1 peptide.
76. An N-terminally acylated peptide or oligopeptide according to any one of the preceding embodiments, which is an absorption enhancer useful for oral delivery of an insulin peptide.
77. An N-terminally acylated peptide or oligopeptide according to any one of the preceding embodiments, which is an absorption enhancer useful for oral delivery of a GLP-1 peptide.
78. An oral pharmaceutical composition comprising an N-terminally acylated peptide or oligopeptide according to any one of the preceding embodiments.
79. An oral pharmaceutical composition according to embodiment 78 further comprising a pharmaceutically active ingredient which is a peptide or protein.
80. An oral pharmaceutical composition according to embodiment 78 further comprising a pharmaceutically active ingredient which is selected from the group consisting of: Insulin peptides and GLP-1 peptides.
81. An oral pharmaceutical composition according to embodiment 78 further comprising a pharmaceutically active ingredient which is an insulin peptide.
82. An oral pharmaceutical composition according to embodiment 78 further comprising a pharmaceutically active ingredient which is a GLP-1 peptide.
83. An oral pharmaceutical composition according to any one of embodiments 78-82, which is a liquid composition.
84. An oral pharmaceutical composition according to any one of embodiments 78-82, which is a solid composition.

### EXAMPLES

The following examples are offered by way of illustration, not by limitation.
The abbreviations used herein are the following:

γGlu: gamma L-glutamyl,

βAsp: beta L-aspartyl,

HCl: hydrochloric acid,

MeCN: acetonitrile,

OEG: [2-(2-aminoethoxy)ethoxy]ethylcarbonyl,

RPC: reverse phase chromatography,

RT: room temperature,

TFA: trifluoroacetic acid,

GI: gastro intestinal,

Fmoc: fluorenylmethyloxycarbonyl,

TRIS: tris(hydroxymethyl)aminomethane,

CH3CN: Acetonitril,

HPLC: High-performance liquid chromatography,

FPLC: Fast protein liquid chromatography,

RP: Reverse phase,

UV: Ultraviolet (light),

LC-MS: Liquid chromatography-mass spectrometry,

NMR: Nuclear magnetic resonance,

TLC: thin layer chromatography,

FRET: Förster resonance energy transfer,

MCA group: 7-methoxycoumarin-4-acetic acid,

DNP: 2,4-dinitrophenol,

GLP-1: Glucagon-like peptide-1,

GI juice: gastro-intestinal juice,

HI: Human insulin,

OtBu: tert-butyl ester,

Pbf: 2,2,4,6,7-pentamethyl-dihydrobenzofuran-5-sulfonyl.

The following examples and general procedures refer to intermediate compounds and final products identified in the specification and in the synthesis schemes. The preparation of the compounds of the present invention is described in detail using the following examples, but the chemical reactions described are disclosed in terms of their general applicability to the preparation of compounds of the invention. Occasionally, the reaction may not be applicable as described to each compound included within the disclosed scope of the invention. The compounds for which this occurs will be readily recognised by those skilled in the art. In these cases the reactions can be successfully performed by conventional modifications known to those skilled in the art, that is, by appropriate protection of interfering groups, by changing to other conventional reagents, or by routine modification of reaction conditions. Alternatively, other reactions disclosed herein or otherwise conventional will be applicable to the preparation of the corresponding compounds of the invention. In all preparative methods, all starting materials are known or may easily be prepared from known starting materials. All temperatures are set forth in degrees Celsius and unless otherwise indicated, all parts and percentages are by weight when referring to yields and all parts are by volume when referring to solvents and eluents.

### Solid Phase Peptide Synthesis—General Procedure 1

This is an example of a synthetic procedure that can be used to prepare oligopeptides of the invention. The exact conditions can be adjusted, for example, the scale of the synthesis can be adjusted to fit the required amounts and/or the resin with intermediate peptide can further be split into several portions followed by the addition of different amino acids to yield different peptides.

### Resin Washing and Coupling of the First Amino Acid.

2-Chlorotrityl resin 100-200 mesh 1.7 mmol/g (2.31 g, 3.93 mmol) was left to swell in dry dichloromethane (12 mL) for 20 min. A solution of Fmoc-protected amino acid (2.62 mmol) and N,N-diisopropylethylamine (1.74 mL, 9.96 mmol) in dry dichloromethane (4 mL) was added to resin and the mixture was shaken for 4 hrs. Resin was filtered and treated with a solution of N,N-diisopropylethylamine (0.91 mL, 5.24 mmol) in methanol/dichloromethane mixture (4:1, 2×20 mL, 2×5 min). Then resin was washed with N,N-dimethylformamide (2×20 mL), dichloromethane (2×20 mL) and N,N-dimethylformamide (3×20 mL). Typical site chain protecting groups were used, for example FMOC-Glu-OtBu, FMOC-Arg-Pbf-OH, FMOC-OEG-OH

Deprotection of the Resin and Coupling of Another Amino Acid (this Step was Repeated Until the Desired Sequence was Assembled on the Resin).

Fmoc group was removed by treatment with 20% piperidine in dimethylformamide (2×20 mL, 1×5 min, 1×30 min). Resin was washed with N,N-dimethylformamide (3×20 mL), 2-propanol (2×20 mL) and dichloromethane (3×20 mL). Solution of Fmoc protected amino acid (3.93 mmol), O-(6-chloro-benzotriazol-1-yl)-N,N,N′,N′-tetramethyluronium tetrafluoroborate (TCTU, 1.40 g, 3.93 mmol) and N,N-diisopropylethylamine (1.23 mL, 7.08 mmol) in N,N-dimethylformamide (10 mL) was added to resin and mixture was shaken for 1 hr. Resin was filtered and washed with N,N-dimethylformamide (2×20 mL), dichloromethane (2×20 mL) and N,N-dimethylformamide (20 mL).

### Deprotection of the Resin and Coupling of Fatty Acid.

Resin was divided in 2 equal parts. One half of the resin (1.31 mmol) was treated with 20% piperidine in dimethylformamide (2×20 mL, 1×5 min, 1×30 min). Resin was washed with N,N-dimethylformamide (3×20 mL), 2-propanol (2×20 mL) and dichloromethane (3×20 mL). Solution of fatty acid (monocarboxylic acid; 3.93 mmol), O-(6-chloro-benzotriazol-1-yl)-N,N,N′,N′-tetramethyluronium tetrafluoroborate (TCTU, 1.40 g, 3.93 mmol) and N,N-diisopropylethylamine (1.23 mL, 7.08 mmol) in dichloromethane/N,N-dimethylformamide mixture (4:1, 10 mL) was added to resin (1.31 mmol) and mixture was shaken for 1 hr. Resin was filtered and washed with N,N-dimethylformamide (3×20 mL), dichloromethane (2×20 mL), methanol (2×20 mL) and dichloromethane (7×20 mL).

Cleavage from the Resin—Method 1.

The product was cleaved from resin by treatment with 2,2,2-trifluoethanol (20 mL) for 18 hrs. Resin was filtered off and washed with dichloromethane (2×20 mL), 2-propanol/dichloromethane mixture (1:1, 2×20 mL), 2-propanol (20 mL) and dichloromethane (3×20 mL). The solvent was removed and hexanes (20 mL) were added to the residue. After stirring for 6 hrs; solid was filtered, washed with hexanes and dried in vacuo to yield the title product as white powder.

Cleavage from the Resin—Method 2.

The product was cleaved from resin (0.74 mmol) by treatment with the mixture of trifluoroacetic acid (9.25 mL), water (250 μL) and triethylsilane (500 μL) for 3 hrs. Resin was filtered off and washed with trifluoroacetic acid (20 mL). Product was precipitated from the solution by the addition of hexanes/diethylether mixture (1:2, 100 mL) and collected by filtration. Product was dissolved in chloroform (30 mL) and the solvent was removed. This procedure was repeated ten times to remove the traces of trifluoroacetic acid. Hexanes/diethylether (50 mL) was added to the residue, formed solid was filtered, washed with hexanes and dried in vacuo.

### Conversion of the Peptide Acid to Sodium Salt.

The peptide acid (275 mg, 357 μmol) was dissolved in 70% aqueous acetonitrile (50 mL) and neutralized with 0.1 M aqueous solution of sodium hydroxide (3.57 mL; the amount of sodium hydroxide was adjusted to fit the number of carboxylic acids in the peptide). Then the solution was freeze-dried to obtain sodium salt of the peptide as fine white powder.

### Parallel Solid Phase Peptide Synthesis—General Procedure 2

To 1 gram of trityl resin (Novabiochem) was coupled 10 eq Fmoc-Tyr(tbu)-OH (Novabiochem) with 1 eq Fmoc-Tyr(3-nitro)-OH in dichloromethane (DCM) and 20 eq of diisopropylamine (DIPEA) for 1 hour. The resin wash washed briefly with NMP and then distributed into a 96 well microtiter filter plate (Nunc). This filter plate was loaded to a Multipep RS instrument from Intavis (Germany). The synthesis steps were allowed to proceed as follows; 1) Deprotection: To each well was added 200 ul of 25% piperidine in NMP for 2+10 min with a multipipette manifold. Then each well was washed with NMP; first 1000 ml then 150 ul three times with multipipette manifold. 2) Coupling step: A given volume of Fmoc-AA-OH as a 0.3 M solution in 0.3M Oxyma Pure solution (Novabiochem) in NMP was preactivated with one-third volume of a 1M diisopropylcarbodiimide (DIC) solution in NMP and one-third volume of a 1M solution of collidine in NMP for 2 min. Then a total of 125 ul of the activated Fmoc-AA-OH was added to each well and allowed to couple for 30 min. This step was repeated twice albeit with the coupling times increased to 60 and 120 min, respectively. The amino acid used in the synthesis were as follows: Fmoc-Ala-OH, Fmoc-Gly-OH, Fmoc-Asn-OH(Novabiochem), Fmoc-Gln-OH(Novabiochem), Fmoc-Arg(Boc)₂-OH (IRIS biotech), Fmoc-Lys(Boc)-OH, Fmoc-Asp(tbu)-OH, Fmoc-Glu(tbu)-OH, Fmoc-His(Boc)-OH, Fmoc-Ser(tbu)-OH, Fmoc-Tyr(tbu)-OH, Fmoc-Tyr(tbu)-OH, Fmoc-Met-OH, Fmoc-Ile-OH, Fmco-Leu-OH, Fmoc-Val-OH, Fmoc-Pro-OH, Fmoc-Phe-OH, Fmoc-Trp(Boc)-OH (all from Protein Technologies unless otherwise indicated). After coupling each well was washed with 300 ul NMP and then three times with 200 ul NMP. The synthesis steps as described above were repeated until the desired length was achieved. Dodecanoic acid was coupled as described above for the Fmoc-amino acids using a 0.3M solution of dodecanoic acid in NMP and activated with one-third volume DIC and one-third volume collidine and then adding 125 ul to each well. The dodecanoic acid was allowed to couple 30 min, 60 min and 120 min (triple couplings). After the addition of the last building block the resin was washed with ethanol and dried.

Cleavage of the peptidyl resin: The dry resin in the 96 well filterplate was placed on top of a 2 ml deepwell polypropylene plate (Nunc). To each well was added 200 ul 95% TFA+5% H₂O (water). in the following intervals: 1 min, 1 min, 15 min, 15 min, 30 min, 30 min. The TFA peptide solution in the deepwell plate was then evaporated to dryness by argon flow. Dry peptides were then dissolved in 80% Dimethyl sulfoxide (DMSO) 20% H2O.

### Purification

Typically, N-terminally acylated peptide or oligopeptides of the invention prepared by solid phase peptide synthesis as described in General procedure 1 have sufficient purity for testing without further purification.

Reversed-phase HPLC purification can be performed as known in the art. Gradient conditions need to be adjusted to the specific compounds as commonly known in the field.

### Anion Exchange

### Typical Purification Procedures:

The HPLC system is a Gilson system consisting of the following: Model 215 Liquid handler, Model 322-H2 Pump and a Model 155 UV Dector. Detection is typically at 210 nm and 280 nm.

The Âkta Purifier FPLC system (GE) consists of the following: Model P-900 Pump, Model UV-900 UV detector, Model pH/C-900 pH and conductivity detector, Model Frac-950 Fraction collector. UV detection is typically at 214 nm, 254 nm and 276 nm.

Acidic HPLC:

Column: Macherey-Nagel SP 250/21 Nucleusil 300-7 C4

Flow: 8 ml/min

Buffer A: 0.1% TFA in acetonitrile

Buffer B: 0.1% TFA in water.

Gradient: 0.0-5.0 min: 10% A

5.00-30.0 min: 10% A to 90% A

30.0-35.0 min: 90% A

35.0-40.0 min: 100% A

Neutral HPLC:

Column: Phenomenex, Jupiter, C4 5 μm 250×10.00 mm, 300 Å

Flow: 6 ml/min

Buffer A: 5 mM TRIS, 7.5 mM (NH₄)₂SO₄, pH=7.3, 20% CH₃CN

Buffer B: 60% CH₃CN, 40% water

Gradient: 0-5 min: 10% B

5-65 min: 10-90% B

65-69 min: 90% B

69-80 min: 90% B

Desalting:

Column: HiPrep 26/10

Flow: 10 ml/min, 6 column volumes

Buffer: 10 mM NH₄HCO₃

### Analysis of the Synthesized Oligopeptides

The identity and purity of the N-terminally modified peptides or oligopeptides of the invention was confirmed by NMR (Bruker AVANCE DPX 200, magnet 300 UltraShield, probe: BBI 300 MHz S1), thin layer chromatography (TLC) and/or LC-MS; a Micromass Quatro micro API mass spectrometer was used to identify the mass of the sample after elution from an HPLC system composed of Waters 2525 binary gradient modul, Waters 2767 sample manager, Waters 2996 Photodiode Array Detector and Waters 2420 ELS Detector. Eluents: A: 0.1% Trifluoro acetic acid in water; B: 0.1% Trifluoro acetic acid in acetonitrile. Column: Sunfire 4.6 mm×100 mm.
The N-terminally modified peptides or oligopeptides of the examples are described as acids, however, when making stock solutions of these compounds in buffer these were converted into salts, such as sodium salt, potassium salt, etcetera.
All N-terminally modified peptides or oligopeptides of examples 1-197 were made according to general procedure 1 or general procedure 2, as listed for each compound.

### Example 1

### N-dodecanoyl-DAla-DAla-DPro-DPhe-OH, General Procedure 1

Alternative name: (R)-2-({(R)-1-[(R)-2-((R)-2-Dodecanoylamino-propionylamino)-propionyl]-pyrrolidine-2-carbonyl}-amino)-3-phenyl-propionic acid.

N-dodecanoyl-DAla-DAla-DPro-DPhe-OH was prepared according to solid phase peptide synthesis—general procedure 1.

¹H-NMR: (300 MHz, AcOD-d4, 80° C., dH): 7.35-7.18 (m, 5H); 4.91 (t, J=6.4 Hz, 1H); 4.78 (m, 1H); 4.62 (m, 2H); 3.87-3.42 (m, 2H); 3.20 (m, 2H); 2.30 (t, J=7.6 Hz, 2H); 2.20-1.88 (m, 4H); 1.64 (m, 2H); 1.46-1.21 (m, 22H); 0.91 (t, J=6.5 Hz, 3H).

LC-MS purity: 100% (ELSD).

LC-MS Rt (Sunfire 4.6 mm×100 mm, acetonitrile/water 70:30 to 100:0+0.1% FA): 3.58 min.

LC-MS m/z: 587.5 (M+H).

TLC: RF (SiO2, dichloromethane/methanol 4:1): 0.20.

### Example 2

### N-tetradecanoyl-DAla-DAla-DPro-DPhe-OH, General Procedure 1

Alternative name: (R)-3-Phenyl-2-({(R)-1-[(R)-2-((R)-2-tetradecanoylamino-propionylamino)-propionyl]-pyrrolidine-2-carbonyl}-amino)-propionic acid.

N-tetradecanoyl-DAla-DAla-DPro-DPhe-OH was prepared according to solid phase peptide synthesis—general procedure 1.

1H-NMR (300 MHz, AcOD-d4, 80° C., dH): 7.34-7.17 (m, 5H); 4.91 (t, J=6.4 Hz, 1H); 4.78 (m, 1H); 4.62 (q, J=6.8 Hz, 2H); 3.87-3.44 (m, 2H); 3.20 (m, 2H); 2.30 (t, J=7.5 Hz, 2H); 2.20-1.89 (m, 4H); 1.64 (m, 2H); 1.43-1.22 (m, 26H); 0.91 (t, J=6.6 Hz, 3H).

LC-MS purity: 100% (ELSD).

LC-MS Rt (Sunfire 4.6 mm×100 mm, acetonitrile/water 70:30 to 100:0+0.1% FA): 5.30 min.

LC-MS m/z: 615.6 (M+H).

TLC: RF (SiO2, dichloromethane/methanol 4:1): 0.20.

### Example 3

### N-tetradecanoyl-Ala-Ala-Pro-Phe-OH, General Procedure 1

Alternative name: (S)-3-Phenyl-2-({(S)-1-[(S)-2-((S)-2-tetradecanoylamino-propionylamino)-propionyl]-pyrrolidine-2-carbonyl}-amino)-propionic acid.

N-tetradecanoyl-Ala-Ala-Pro-Phe-OH was prepared according to solid phase peptide synthesis—general procedure 1.

1H-NMR (300 MHz, AcOD-d4, 80° C., dH): 7.34-7.17 (m, 5H); 4.91 (t, J=6.4 Hz, 1H); 4.78 (m, 1H); 4.62 (m, 2H); 3.87-3.44 (m, 2H); 3.20 (m, 2H); 2.30 (t, J=7.5 Hz, 2H); 2.20-1.89 (m, 4H); 1.64 (m, 2H); 1.43-1.22 (m, 26H); 0.91 (t, J=6.6 Hz, 3H).

LC-MS purity: 97% (ELSD).

LC-MS Rt (Sunfire 4.6 mm×100 mm, acetonitrile/water 70:30 to 100:0+0.1% FA): 5.22 min.

LC-MS m/z: 615.5 (M+H).

TLC: RF (SiO2, chloroform/methanol 4:1): 0.50.

### Example 4

### N-dodecanoyl-Ala-Ala-Pro-DPhe-OH, General Procedure 1

Alternative name: (R)-2-({(S)-1-[(S)-2-((S)-2-Dodecanoylamino-propionylamino)-propionyl]-pyrrolidine-2-carbonyl}-amino)-3-phenyl-propionic acid.

N-dodecanoyl-Ala-Ala-Pro-DPhe-OH was prepared according to solid phase peptide synthesis—general procedure 1.

1H-NMR (300 MHz, AcOD-d4, 80° C., dH): 7.37-7.14 (m, 5H); 4.92 (dd, J=7.8 Hz, 5.4, 1H); 4.76 (d, J=6.4 Hz, 1H); 4.61 (d, J=6.8 Hz, 1H); 3.76 (bs, 1H); 3.62 (bs, 1H); 3.37-3.14 (m, 1H); 3.07 (bs, 1H); 2.29 (t, J=7.6 Hz, 2H); 2.03-1.85 (m, 4H); 1.63 (m, 2H); 1.48-1.20 (m, 22H); 0.91 (t, J=6.6 Hz, 3H).

LC-MS purity: 100% (ELSD).

LC-MS Rt (Sunfire 4.6 mm×100 mm, acetonitrile/water 70:30 to 100:0+0.1% FA): 3.66 min.

LC-MS m/z: 587.5 (M+H).

TLC: RF (SiO2, dichloromethane/methanol 4:1): 0.20.

### Example 5

### N-tetradecanoyl-Ala-Ala-Pro-DPhe-OH, General Procedure 1

Alternative name: (R)-3-Phenyl-2-({(S)-1-[(S)-2-((S)-2-tetradecanoylamino-propionylamino)-propionyl]-pyrrolidine-2-carbonyl}-amino)-propionic acid.

N-tetradecanoyl-Ala-Ala-Pro-DPhe-OH was prepared according to solid phase peptide synthesis—general procedure 1.

1H-NMR (300 MHz, AcOD-d4, 80° C., dH): 7.35-7.15 (m, 5H); 4.92 (dd, J=7.9, 5.5 Hz, 1H); 4.76 (d, J=7.2 Hz, 1H); 4.61 (d, J=7.0 Hz, 2H); 3.76 (bs, 1H); 3.62 (bs, 1H); 3.36-3.19 (m, 1H); 3.07 (bs, 1H); 2.29 (t, J=7.5 Hz, 2H); 2.01-1.86 (m, 4H); 1.63 (m, 2H); 1.46-1.23 (m, 26H); 0.91 (t, J=6.6 Hz, 3H).

LC-MS purity: 100% (ELSD).

LC-MS Rt (Sunfire 4.6 mm×100 mm, acetonitrile/water 70:30 to 100:0+0.1% FA): 5.24 min.

LC-MS m/z: 615.6 (M+H).

TLC: RF (SiO2, dichloromethane/methanol 4:1): 0.20. (1A)

### Example 6

### N-dodecanoyl-βAla-βAla-Pro-Phe-Pro-OH, General Procedure 1

Alternative name: (S)-1-[(S)-2-({(S)-1-[3-(3-Dodecanoylamino-propionylamino)-propionyl]-pyrrolidine-2-carbonyl}-amino)-3-phenyl-propionyl]-pyrrolidine-2-carboxylic acid.

N-dodecanoyl-βAla-βAla-Pro-Phe-Pro-OH was prepared according to solid phase peptide synthesis—general procedure 1.

1H-NMR (300 MHz, AcOD-d4, 80° C., dH): 7.40-7.18 (m, 5H); 5.21-4.90 (m, 1H); 4.67-4.37 (m, 2H); 3.98-3.35 (m, 8H); 3.09 (m, 2H); 2.63 (m, 2H); 2.51 (t, J=5.3 Hz, 2H); 2.26 (t, J=7.6 Hz, 2H); 2.18-1.86 (m, 8H); 1.63 (m, 2H); 1.32 (bs, 16H); 0.91 (t, J=6.7 Hz, 3H).

LC-MS purity: 98% (ELSD).

LC-MS Rt (Sunfire 4.6 mm×100 mm, acetonitrile/water 70:30 to 100:0+0.1% FA): 2.66 min.

LC-MS m/z: 684.6 (M+H).

TLC: RF (SiO2, dichloromethane/methanol 4:1): 0.25.

### Example 7

### N-dodecanoyl-Aib-Aib-Pro-Phe-OH, General Procedure 1

Alternative name: (S)-2-({(S)-1-[2-(2-Dodecanoylamino-2-methyl-propionylamino)-2-methyl-propionyl]-pyrrolidine-2-carbonyl}-amino)-3-phenyl-propionic acid.

N-dodecanoyl-Aib-Aib-Pro-Phe-OH was prepared according to solid phase peptide synthesis—general procedure 1.

1H-NMR (300 MHz, AcOD-d4, 80° C., dH): 7.29-7.21 (m, 5H); 4.88 (m, 1H); 4.64 (m, 1H); 3.58-3.60 (m, 2H); 3.40-3.08 (m, 2H); 2.29 (t, J=7.6 Hz, 2H); 1.81 (m, 2H); 1.75-1.60 (m, 4H); 1.57 (d, J=13.5 Hz, 6H); 1.51 (d, J=13.5 Hz, 6H); 1.30 (m, 16H); 0.90 (t, J=6.5 Hz, 3H).

LC-MS purity: 100% (ELSD).

LC-MS Rt (Sunfire 4.6 mm×100 mm, acetonitrile/water 70:30 to 100:0+0.1% FA): 4.70 min.

LC-MS m/z: 615.4 (M+H).

TLC: RF (SiO2, dichloromethane/methanol 4:1): 0.20.

### Example 8

### N-dodecanoyl-βAla-Ala-Pro-Phe-OH, General Procedure 1

Alternative name: (S)-2-({(S)-1-[(S)-2-(3-Dodecanoylamino-propionylamino)-propionyl]-pyrrolidine-2-carbonyl}-amino)-3-phenyl-propionic acid.

N-dodecanoyl-βAla-Ala-Pro-Phe-OH was prepared according to solid phase peptide synthesis—general procedure 1.

1H-NMR (300 MHz, AcOD-d4, 80° C., dH): 7.29-7.21 (m, 5H); 4.90 (dd, J1=J2=6.3 Hz, 1H); 4.79 (m, 1H); 4.63 (m, 1H); 3.85-3.60 (m, 2H); 3.53 (t, J=6.2 Hz, 2H); 3.30-3.08 (m, 2H); 2.54 (t, J=6.2 Hz, 2H); 2.26 (t, J=7.6 Hz, 2H); 2.11 (m, 2H); 1.93 (m, 2H); 1.63 (m, 2H); 1.32 (m, 19H); 0.91 (t, J=6.5 Hz, 3H).

LC-MS purity: 99% (ELSD).

LC-MS Rt (Sunfire 4.6 mm×100 mm, acetonitrile/water 70:30 to 100:0+0.1% FA): 2.90 min.

LC-MS m/z: 587.3 (M+H).

### Example 9

### N-tetradecanoyl-βAla-Ala-Pro-Phe-OH, General Procedure 1

Alternative name: (S)-3-Phenyl-2-({(S)-1-[(S)-2-(3-tetradecanoylamino-propionylamino)-propionyl]-pyrrolidine-2-carbonyl}-amino)-propionic acid.

N-tetradecanoyl-βAla-Ala-Pro-Phe-OH was prepared according to solid phase peptide synthesis—general procedure 1.

1H-NMR (300 MHz, AcOD-d4, 80° C., dH): 7.29-7.21 (m, 5H); 4.91 (dd, J1=J2=6.3 Hz, 1H); 4.79 (m, 1H); 4.63 (m, 1H); 3.82-3.60 (m, 2H); 3.53 (t, J=6.2 Hz, 2H); 3.30-3.08 (m, 2H); 2.53 (t, J=6.2 Hz, 2H); 2.26 (t, J=7.6 Hz, 2H); 2.11 (m, 2H); 1.93 (m, 2H); 1.62 (m, 2H); 1.32 (m, 21H); 0.91 (t, J=6.5 Hz, 3H).

LC-MS purity: 100% (ELSD).

LC-MS Rt (Sunfire 4.6 mm×100 mm, acetonitrile/water 70:30 to 100:0+0.1% FA): 4.35 min.

LC-MS m/z: 615.4 (M+H).

TLC: RF (SiO2, dichloromethane/methanol 4:1): 0.20.

### Example 10

### N-dodecanoyl-βAla-βAla-Pro-Phe-OH, General Procedure 1

Alternative name: (S)-2-({(S)-1-[3-(3-Dodecanoylamino-propionylamino)-propionyl]-pyrrolidine-2-carbonyl}-amino)-3-phenyl-propionic acid.

N-dodecanoyl-βAla-βAla-Pro-Phe-OH was prepared according to solid phase peptide synthesis—general procedure 1.

1H-NMR (300 MHz, AcOD-d4, 80° C., dH): 7.29-7.21 (m, 5H); 4.92 (m, 1H); 4.52 (m, 1H); 3.53 (m, 6H); 3.30-3.08 (m, 2H); 2.62 (t, J=5.7 Hz, 2H); 2.50 (t, J=6.1 Hz, 2H); 2.26 (t, J=7.6 Hz, 2H); 2.07 (m, 2H); 1.93 (m, 2H); 1.62 (m, 2H); 1.32 (m, 16H); 0.91 (t, J=6.5 Hz, 3H).

LC-MS purity: 100% (ELSD).

LC-MS Rt (Sunfire 4.6 mm×100 mm, acetonitrile/water 70:30 to 100:0+0.1% FA): 3.15 min.

LC-MS m/z: 587.3 (M+H).

TLC: RF (SiO2, dichloromethane/methanol 4:1): 0.20.

### Example 11

### N-dodecanoyl-Ala-Ala-Pro-Leu-OH, General Procedure 1

Alternative name: (S)-2-({(S)-1-[(S)-2-((S)-2-Dodecanoylamino-propionylamino)-propionyl]-pyrrolidine-2-carbonyl}-amino)-4-methyl-pentanoic acid.

N-dodecanoyl-Ala-Ala-Pro-Leu-OH was prepared according to solid phase peptide synthesis—general procedure 1.

1H-NMR (300 MHz, AcOD-d4, 80° C., dH): 4.81 (m, 1H); 4.68-4.56 (m, 3H); 3.89-3.60 (m, 2H); 2.29 (t, J=7.5 Hz, 2H); 2.23-1.92 (m, 4H); 1.86-1.56 (m, 5H); 1.45-1.22 (m, 22H); 0.97 (t, J=6.5 Hz, 6H); 0.91 (t, J=6.7 Hz, 3H).

LC-MS purity: 100% (ELSD).

LC-MS Rt (Sunfire 4.6 mm×100 mm, acetonitrile/water 50:50 to 100:0+0.1% FA): 5.35 min.

LC-MS m/z: 553.5 (M+H).

TLC: RF (SiO2, dichloromethane/methanol 4:1): 0.20.

### Example 12

### N-dodecanoyl-γGlu-Ala-Ala-Pro-Phe-OH, General Procedure 1

Alternative name: (S)-4-((S)-1-{(S)-2-[(S)-2-((S)-1-Carboxy-2-phenyl-ethylcarbamoyl)-pyrrolidin-1-yl]-1-methyl-2-oxo-ethylcarbamoyl}-ethylcarbamoyl)-2-dodecanoylamino-butyric acid.

N-dodecanoyl-γGlu-Ala-Ala-Pro-Phe-OH was prepared according to solid phase peptide synthesis—general procedure 1.

1H-NMR (300 MHz, AcOD-d4, 80° C., dH): 7.37-7.19 (m, 5H); 4.91 (t, J=6.2 Hz, 1H); 4.78 (bs, 1H); 4.70-4.52 (m, 3H); 3.78 (bs, 1H); 3.63 (bs, 1H); 3.55-3.10 (m, 2H); 2.45 (t, J=7.3 Hz, 2H); 2.34 (t, J=7.6 Hz, 2H); 2.34-1.85 (m, 6H); 1.74-1.57 (m, 2H); 1.46-1.21 (m, 22H); 0.91 (t, J=6.5 Hz, 3H).

LC-MS purity: 95% (ELSD)

LC-MS Rt (Sunfire 4.6 mm×100 mm, acetonitrile/water 35:65 to 100:0+0.1% FA): 6.54 min.

LC-MS m/z: 716.6 (M+H).

TLC: RF (SiO2, dichloromethane/methanol 4:1): 0.10.

### Example 13

### N-tetradecanoyl-Glu-Ala-Ala-Pro-Phe-OH, General Procedure 1

Alternative name: (S)-4-((S)-1-{(S)-2-[(S)-2-((S)-1-Carboxy-2-phenyl-ethylcarbamoyl)-pyrrolidin-1-yl]-1-methyl-2-oxo-ethylcarbamoyl}-ethylcarbamoyl)-2-tetradecanoylamino-butyric acid.

N-tetradecanoyl-Glu-Ala-Ala-Pro-Phe-OH was prepared according to solid phase peptide synthesis—general procedure 1.

1H-NMR300 MHz, AcOD-d4, 80° C., dH): 7.37-7.19 (m, 5H); 4.92 (t, J=6.2 Hz, 1H); 4.79 (bs, 1H); 4.71-4.53 (m, 3H); 3.80 (bs, 1H); 3.65 (bs, 1H); 3.55-3.20 (m, 2H); 2.47 (t, J=8.3 Hz, 2H); 2.35 (t, J=7.5 Hz, 2H); 2.31-2.06 (m, 6H); 1.74-1.57 (m, 2H); 1.46-1.21 (m, 24H); 0.91 (t, J=6.5 Hz, 3H).

LC-MS purity: 100% (ELSD)

LC-MS Rt (Sunfire 4.6 mm×100 mm, acetonitrile/water 35:65 to 100:0+0.1% FA): 7.75 min.

LC-MS m/z: 744.4 (M+H).

### Example 14

### Ala-Ala-Pro-Phe-OH, General Procedure 1

Alternative name: (S)-2-({(S)-1-[(S)-2-((S)-2-Amino-propionylamino)-propionyl]-pyrrolidine-2-carbonyl}-amino)-3-phenyl-propionic acid.

Ala-Ala-Pro-Phe-OH was prepared according to solid phase peptide synthesis—general procedure 1.

1H-NMR (300 MHz, AcOD-d4, 80° C., dH): 7.34-7.17 (m, 5H); 4.91 (t, J=6.4 Hz, 1H); 4.81 (m, 1H); 4.62 (m, 1H); 4.29 (m, 1H); 3.87-3.44 (m, 2H); 3.19 (m, 2H); 2.26-1.96 (m, 4H); 1.58 (m, 3H); 1.38 (m, 3H).

LC-MS purity: 100% (ELSD).

LC-MS Rt (Sunfire 4.6 mm×100 mm, acetonitrile/water 5:95 to 100:0+0.1% FA): 4.84 min.

LC-MS m/z: 405.1 (M+H).

TLC: RF (SiO2, chloroform/methanol 4:1): 0.05.

### Example 15

### N-dodecandioyl-Ala-Ala-Pro-Phe-OH, General Procedure 1

Alternative name: 11-((S)-1-{(S)-2-[(S)-2-((S)-1-Carboxy-2-phenyl-ethylcarbamoyl)-pyrrolidin-1-yl]-1-methyl-2-oxo-ethylcarbamoyl}-ethylcarbamoyl)-undecanoic acid.

N-dodecandioyl-Ala-Ala-Pro-Phe-OH was prepared according to solid phase peptide synthesis—general procedure 1.

1H-NMR (300 MHz, AcOD-d4, 80° C., dH): 7.34-7.16 (m, 5H); 4.91 (t, J=6.4 Hz, 1H); 4.78 (m, 1H); 4.61 (m, 2H); 3.87-3.44 (m, 2H); 3.20 (m, 2H); 2.33 (m, 4H); 2.17-1.90 (m, 4H); 1.64 (m, 4H); 1.46-1.22 (m, 18H).

LC-MS purity: 100% (ELSD).

LC-MS Rt (Sunfire 4.6 mm×100 mm, acetonitrile/water 35:65 to 100:0+0.1% FA): 4.67 min.

LC-MS m/z: 617.3 (M+H).

TLC: RF (SiO2, chloroform/methanol 4:1): 0.50.

### Example 16

### N-tetradecandioyl-Ala-Ala-Pro-Phe-OH, General Procedure 1

Alternative name: 13-((S)-1-{(S)-2-[(S)-2-((S)-1-Carboxy-2-phenyl-ethylcarbamoyl)-pyrrolidin-1-yl]-1-methyl-2-oxo-ethylcarbamoyl}-ethylcarbamoyl)-tridecanoic acid.

N-tetradecandioyl-Ala-Ala-Pro-Phe-OH was prepared according to solid phase peptide synthesis—general procedure 1.

1H-NMR (300 MHz, AcOD-d4, 80° C., dH): 7.34-7.15 (m, 5H); 4.91 (t, J=6.4 Hz, 1H); 4.78 (m, 1H); 4.61 (m, 2H); 3.88-3.49 (m, 2H); 3.20 (m, 2H); 2.33 (m, 4H); 2.20-1.94 (m, 4H); 1.64 (m, 4H); 1.45-1.24 (m, 22H).

LC-MS purity: 100% (ELSD).

LC-MS Rt (Sunfire 4.6 mm×100 mm, acetonitrile/water 35:65 to 100:0+0.1% FA): 5.35 min.

LC-MS m/z: 645.3 (M+H).

TLC: RF (SiO2, chloroform/methanol 4:1): 0.45.

### Example 17

### N-dodecanoyl-Ala-Ala-Pro-Tyr-OH, General Procedure 1

N-dodecanoyl-Ala-Ala-Pro-Tyr-OH was prepared according to solid phase peptide synthesis—general procedure 1.

### Example 18

### N-dodecanoyl-Ala-Ala-Ala-Pro-Phe-OH, General Procedure 1

N-dodecanoyl-Ala-Ala-Ala-Pro-Phe-OH was prepared according to solid phase peptide synthesis—general procedure 1.

### Example 19

### N-dodecanoyl-Ala-Ala-Ala-Ala-Pro-Phe-OH, General Procedure 1

N-dodecanoyl-Ala-Ala-Ala-Ala-Pro-Phe-OH was prepared according to solid phase peptide synthesis—general procedure 1.

### Example 20

### N-decanoyl-Ala-Ala-Pro-Arg-OH, General Procedure 1

N-decanoyl-Ala-Ala-Pro-Arg-OH was prepared according to solid phase peptide synthesis—general procedure 1.

### Example 21

### N-dodecanoyl-γGlu-Ala-Pro-Arg-OH, General Procedure 1

N-dodecanoyl-γGlu-Ala-Pro-Arg-OH was prepared according to solid phase peptide synthesis—general procedure 1.

### Example 22

### N-dodecanoyl-γGlu-Ala-Pro-Phe-OH, General Procedure 1

N-dodecanoyl-γGlu-Ala-Pro-Phe-OH was prepared according to solid phase peptide synthesis—general procedure 1.

### Example 23

### N-tetradecanoyl-γGlu-Ala-Pro-Phe-OH, General Procedure 1

N-tetradecanoyl-γGlu-Ala-Pro-Phe-OH was prepared according to solid phase peptide synthesis—general procedure 1.

### Example 24

### N-dodecanoyl-Ala-Ala-Pro-Phe-Pro-OH, General Procedure 1

N-dodecanoyl-Ala-Ala-Pro-Phe-Pro-OH was prepared according to solid phase peptide synthesis—general procedure 1.

### Example 25

### N-dodecanoyl-γGlu-Ala-Ala-Pro-Arg-OH, General Procedure 1

N-dodecanoyl-γGlu-Ala-Ala-Pro-Arg-OH was prepared according to solid phase peptide synthesis—general procedure 1.

### Example 26

### N-dodecanoyl-Ala-Ala-Pro-Trp-OH, General Procedure 1

N-dodecanoyl-Ala-Ala-Pro-Trp-OH was prepared according to solid phase peptide synthesis—general procedure 1.

### Example 27

### N-dodecanoyl-γGlu-Ala-Ala-Pro-Arg-Pro-OH, General Procedure 1

N-dodecanoyl-γGlu-Ala-Ala-Pro-Arg-Pro-OH was prepared according to solid phase peptide synthesis—general procedure 1.

### Example 28

### N-eicosanoyl-Ala-Ala-Pro-Phe-OH, General Procedure 1

Alternative name: (S)-2-({(S)-1-[(S)-2-((S)-2-Icosanoylamino-propionylamino)-propionyl]-pyrrolidine-2-carbonyl}-amino)-3-phenyl-propionic acid.

N-eicosanoyl-Ala-Ala-Pro-Phe-OH was prepared according to solid phase peptide synthesis—general procedure 1.

1H-NMR (300 MHz, CDCl3, dH): 8.14 and 7.77 (d, J=7.9 and 7.4 Hz, 1H); 7.36-6.98 (m, 5H); 6.77-6.46 (m, 1H); 4.86-4.21 (m, 4H); 3.74-3.00 (m, 4H); 2.36-2.12 (m, 3H); 2.09-1.81 (m, 3H); 1.70-1.51 (m, 2H); 1.39-1.10 (m, 38H); 0.89 (t, J=6.6 Hz, 3H).

(300 MHz, CDCl3, H): 8.16 and 7.74 (d, J=7.9 and 7.4 Hz, 1H); 7.33-7.00 (m, 5H); 6.68-6.42 (m, 2H); 4.84-4.23 (m, 4H); 3.74-3.03 (m, 4H); 2.33-2.14 (m, 3H); 2.07-1.89 (m, 3H); 1.67-1.53 (m, 2H); 1.42-1.10 (m, 30H); 0.88 (t, J=6.6 Hz, 3H).

LC-MS purity: 100% (ELSD).

LC-MS Rt (Sunfire 4.6 mm×100 mm, acetonitrile/water 95:5 to 100:0+0.1% FA): 11.38 min.

LC-MS m/z: 699.5 (M+H).

LC-MS purity: 97% (ELSD).

LC-MS Rt (Sunfire 4.6 mm×100 mm, acetonitrile/water 95:5 to 100:0+0.1% FA): 5.19 min.

LC-MS m/z: 643.4 (M+H).

### Example 29

### N-hexadecanoyl-Ala-Ala-Pro-Phe-OH, General Procedure 1

Alternative name: (S)-2-({(S)-1-[(S)-2-((S)-2-Hexadecanoylamino-propionylamino)-propionyl]-pyrrolidine-2-carbonyl}-amino)-3-phenyl-propionic acid.

N-hexadecanoyl-Ala-Ala-Pro-Phe-OH was prepared according to solid phase peptide synthesis—general procedure 1.

### Example 30

### N-octadecanoyl-Ala-Ala-Pro-Phe-OH, General Procedure 1

Alternative name: (S)-2-({(S)-1-[(S)-2-((S)-2-Octadecanoylamino-propionylamino)-propionyl]-pyrrolidine-2-carbonyl}-amino)-3-phenyl-propionic acid.

N-octadecanoyl-Ala-Ala-Pro-Phe-OH was prepared according to solid phase peptide synthesis—general procedure 1.

1H-NMR (300 MHz, AcOD-d4, dH): 7.37-7.14 (m, 5H); 4.93 (t, J=6.0 Hz, 1H); 4.77 and 4.37 (q and m, J=7.0 Hz, 1H); 4.67-4.54 (m, 2H); 4.43-4.33 (m, 1H); 3.91-3.05 (m, 4H); 2.28 (t, J=7.6 Hz, 2H); 2.20-1.92 (m, 6H); 1.68-1.52 (m, 2H); 1.45-1.20 (m, 34H); 0.96-0.84 (m, 3H).

LC-MS purity: 100% (ELSD).

LC-MS Rt (Sunfire 4.6 mm×100 mm, acetonitrile/water 95:5 to 100:0+0.1% FA): 5.52 min.

LC-MS m/z: 671.4 (M+H).

### Example 31

### N-tetradecanoyl-Arg-Ala-Ala-Pro-Phe-OH, General Procedure 1

Alternative name: (S)-2-[((S)-1-{(S)-2-[(S)-2-((S)-5-Guanidino-2-tetradecanoylamino-pentanoylamino)-propionylamino]-propionyl}-pyrrolidine-2-carbonyl)amino]-3-phenyl-propionic acid.

N-tetradecanoyl-Arg-Ala-Ala-Pro-Phe-OH was prepared according to solid phase peptide synthesis—general procedure 1.

1H-NMR (300 MHz, AcOD, 80° C., dH): 7.36-7.16 (m, 5H); 4.97-4.40 (m, 5H); 3.89-3.43 (m, 2H); 3.38-3.05 (m, 4H); 2.42-2.21 (m, 2H); 2.20-1.54 (m, 10H); 1.49-1.03 (m, 28H); 0.98-0.81 (m, 3H).

LC-MS purity: 100% (ELSD).

LC-MS Rt (Sunfire 4.6 mm×100 mm, acetonitrile/water 35:65 to 100:0+0.1% FA): 4.52 min.

LC-MS m/z: 771.5 (M+H).

### Example 32

### N-hexadecanoyl-γGlu-Ala-Ala-Pro-Phe-OH, General Procedure 1

Alternative name: (S)-4-((S)-1-{(S)-2-[(S)-2-((S)-1-Carboxy-2-phenyl-ethylcarbamoyl)-pyrrolidin-1-yl]-1-methyl-2-oxo-ethylcarbamoyl}-ethylcarbamoyl)-2-hexadecanoylamino-butyric acid.

N-hexadecanoyl-γGlu-Ala-Ala-Pro-Phe-OH was prepared according to solid phase peptide synthesis—general procedure 1.

1H-NMR (300 MHz, AcOD, 80° C., dH): 7.35-7.18 (m, 5H); 4.97-4.47 (m, 5H); 3.89-3.45 (m, 2H); 3.37-3.04 (m, 2H); 2.55-1.96 (m, 10H); 1.76-1.58 (m, 2H); 1.47-1.11 (m, 30H); 0.97-0.83 (m, 3H).

LC-MS purity: 100% (ELSD).

LC-MS Rt (Sunfire 4.6 mm×100 mm, acetonitrile/water 70:30 to 100:0+0.1% FA):

6.00 min.

LC-MS m/z: 772.4 (M+H).

### Example 33

### N-decanoyl-Ala-Ala-Pro-Phe-OH, General Procedure 1

Alternative name: N-decanoyl-Ala-Ala-Pro-Phe-OH was prepared according to solid phase peptide synthesis—general procedure 1.
Example 34

### N-dodecanoyl-Ala-Ala-Pro-Phe-OH, General Procedure 1

Alternative name: (S)-2-({(S)-1-[(S)-2-((S)-2-Dodecanoylamino-propionylamino)-propionyl]-pyrrolidine-2-carbonyl}-amino)-3-phenyl-propionic acid.

N-dodecanoyl-Ala-Ala-Pro-Phe-OH was prepared according to solid phase peptide synthesis—general procedure 1.

1H-NMR (300 MHz, AcOD-d4, 80° C., dH): 7.39-7.11 (m, 5H); 4.91 (t, J=6.2 Hz, 1H); 4.78 (m, 1H); 4.62 (m, 2H); 3.87-3.42 (m, 2H); 3.20 (m, 2H); 2.30 (t, J=7.6 Hz, 2H); 2.20-1.88 (m, 4H); 1.64 (m, 2H); 1.46-1.21 (m, 22H); 0.91 (t, J=6.5 Hz, 3H).

LC-MS purity: 100% (ELSD).

LC-MS Rt (Sunfire 4.6 mm×100 mm, acetonitrile/water 70:30 to 100:0+0.1% FA): 3.58 min.

LC-MS m/z: 587.3 (M+H).

TLC: RF (SiO2, chloroform/methanol 4:1): 0.70.

### Example 35

### N-dodecanoyl-Ala-Pro-Phe-OH, General Procedure 1

N-dodecanoyl-Ala-Pro-Phe-OH was prepared according to solid phase peptide synthesis—general procedure 1.

### Example 36

### N-dodecanoyl-Gly-Ala-Ala-Pro-Tyr-OH, General Procedure 2

N-dodecanoyl-Gly-Ala-Ala-Pro-Tyr-OH was prepared according to solid phase peptide synthesis—general procedure 2.

### Example 37

### N-dodecanoyl-Gly-Ala-Pro-Tyr-OH, General Procedure 2

N-dodecanoyl-Gly-Ala-Pro-Tyr-OH was prepared according to solid phase peptide synthesis—general procedure 2.

### Example 38

### N-dodecanoyl-His-Ala-Ala-Pro-Tyr-OH, General Procedure 2

N-dodecanoyl-His-Ala-Ala-Pro-Tyr-OH was prepared according to solid phase peptide synthesis—general procedure 2.

### Example 39

### N-dodecanoyl-His-Ala-Pro-Tyr-OH, General Procedure 2

N-dodecanoyl-His-Ala-Pro-Tyr-OH was prepared according to solid phase peptide synthesis—general procedure 2.

### Example 40

### N-dodecanoyl-Ile-Ala-Ala-Pro-Tyr-OH, General Procedure 2

N-dodecanoyl-Ile-Ala-Ala-Pro-Tyr-OH was prepared according to solid phase peptide synthesis—general procedure 2.

### Example 41

### N-dodecanoyl-Ile-Ala-Pro-Tyr-OH, General Procedure 2

N-dodecanoyl-Ile-Ala-Pro-Tyr-OH was prepared according to solid phase peptide synthesis—general procedure 2.

### Example 42

### N-dodecanoyl-Leu-Ala-Ala-Pro-Tyr-OH, General Procedure 2

N-dodecanoyl-Leu-Ala-Ala-Pro-Tyr-OH was prepared according to solid phase peptide synthesis—general procedure 2.

### Example 43

### N-dodecanoyl-Leu-Ala-Pro-Tyr-OH, General Procedure 2

N-dodecanoyl-Leu-Ala-Pro-Tyr-OH was prepared according to solid phase peptide synthesis—general procedure 2.

### Example 44

### N-dodecanoyl-Lys-Ala-Ala-Pro-Tyr-OH, General Procedure 2

N-dodecanoyl-Lys-Ala-Ala-Pro-Tyr-OH was prepared according to solid phase peptide synthesis—general procedure 2.

### Example 45

### N-dodecanoyl-Lys-Ala-Pro-Tyr-OH, General Procedure 2

N-dodecanoyl-Lys-Ala-Pro-Tyr-OH was prepared according to solid phase peptide synthesis—general procedure 2.

### Example 46

### N-dodecanoyl-Met-Ala-Ala-Pro-Tyr-OH, General Procedure 2

N-dodecanoyl-Met-Ala-Ala-Pro-Tyr-OH was prepared according to solid phase peptide synthesis—general procedure 2.

### Example 47

### N-dodecanoyl-Met-Ala-Pro-Tyr-OH, General Procedure 2

N-dodecanoyl-Met-Ala-Pro-Tyr-OH was prepared according to solid phase peptide synthesis—general procedure 2.

### Example 48

### N-dodecanoyl-Pro-Ala-Ala-Pro-Tyr-OH, General Procedure 2

N-dodecanoyl-Pro-Ala-Ala-Pro-Tyr-OH was prepared according to solid phase peptide synthesis—general procedure 2.

### Example 49

### N-dodecanoyl-Pro-Ala-Pro-Tyr-OH, General Procedure 2

N-dodecanoyl-Pro-Ala-Pro-Tyr-OH was prepared according to solid phase peptide synthesis—general procedure 2.

### Example 50

### N-dodecanoyl-Ser-Ala-Ala-Pro-Tyr-OH, General Procedure 2

N-dodecanoyl-Ser-Ala-Ala-Pro-Tyr-OH was prepared according to solid phase peptide synthesis—general procedure 2.

### Example 51

### N-dodecanoyl-Ser-Ala-Pro-Tyr-OH, General Procedure 2

N-dodecanoyl-Ser-Ala-Pro-Tyr-OH was prepared according to solid phase peptide synthesis—general procedure 2.

### Example 52

### N-dodecanoyl-Thr-Ala-Ala-Pro-Tyr-OH, General Procedure 2

N-dodecanoyl-Thr-Ala-Ala-Pro-Tyr-OH was prepared according to solid phase peptide synthesis—general procedure 2.

### Example 53

### N-dodecanoyl-Thr-Ala-Pro-Tyr-OH, General Procedure 2

N-dodecanoyl-Thr-Ala-Pro-Tyr-OH was prepared according to solid phase peptide synthesis—general procedure 2.

### Example 54

### N-dodecanoyl-Val-Ala-Ala-Pro-Tyr-OH, General Procedure 2

N-dodecanoyl-Val-Ala-Ala-Pro-Tyr-OH was prepared according to solid phase peptide synthesis—general procedure 2.

### Example 55

### N-dodecanoyl-Val-Ala-Pro-Tyr-OH, General Procedure 2

N-dodecanoyl-Val-Ala-Pro-Tyr-OH was prepared according to solid phase peptide synthesis—general procedure 2.

### Example 56

### N-dodecanoyl-Ala-Ala-Ala-Pro-Tyr-OH, General Procedure 2

N-dodecanoyl-Ala-Ala-Ala-Pro-Tyr-OH was prepared according to solid phase peptide synthesis—general procedure 2.

### Example 57

### N-dodecanoyl-Ala-Ala-Ala-Tyr-OH, General Procedure 2

N-dodecanoyl-Ala-Ala-Ala-Tyr-OH was prepared according to solid phase peptide synthesis—general procedure 2.

### Example 58

### N-dodecanoyl-Ala-Ala-Arg-Tyr-OH, General Procedure 2

N-dodecanoyl-Ala-Ala-Arg-Tyr-OH was prepared according to solid phase peptide synthesis—general procedure 2.

### Example 59

### N-dodecanoyl-Ala-Ala-Asn-Tyr-OH, General Procedure 2

N-dodecanoyl-Ala-Ala-Asn-Tyr-OH was prepared according to solid phase peptide synthesis—general procedure 2.

### Example 60

### N-dodecanoyl-Ala-Ala-Asp-Tyr-OH, General Procedure 2

N-dodecanoyl-Ala-Ala-Asp-Tyr-OH was prepared according to solid phase peptide synthesis—general procedure 2.

### Example 61

### N-dodecanoyl-Ala-Ala-Gln-Tyr-OH, General Procedure 2

N-dodecanoyl-Ala-Ala-Gln-Tyr-OH was prepared according to solid phase peptide synthesis—general procedure 2.

### Example 62

### N-dodecanoyl-Ala-Ala-Glu-Tyr-OH, General Procedure 2

N-dodecanoyl-Ala-Ala-Glu-Tyr-OH was prepared according to solid phase peptide synthesis—general procedure 2.

### Example 63

### N-dodecanoyl-Ala-Ala-Gly-Tyr-OH, General Procedure 2

N-dodecanoyl-Ala-Ala-Gly-Tyr-OH was prepared according to solid phase peptide synthesis—general procedure 2.

### Example 64

### N-dodecanoyl-Ala-Ala-His-Tyr-OH, General Procedure 2

N-dodecanoyl-Ala-Ala-His-Tyr-OH was prepared according to solid phase peptide synthesis—general procedure 2.

### Example 65

### N-dodecanoyl-Ala-Ala-Ile-Tyr-OH, General Procedure 2

N-dodecanoyl-Ala-Ala-Ile-Tyr-OH was prepared according to solid phase peptide synthesis—general procedure 2.

### Example 66

### N-dodecanoyl-Ala-Ala-Leu-Tyr-OH, General Procedure 2

N-dodecanoyl-Ala-Ala-Leu-Tyr-OH was prepared according to solid phase peptide synthesis—general procedure 2.

### Example 67

### N-dodecanoyl-Ala-Ala-Lys-Tyr-OH, General Procedure 2

N-dodecanoyl-Ala-Ala-Lys-Tyr-OH was prepared according to solid phase peptide synthesis—general procedure 2.

### Example 68

### N-dodecanoyl-Ala-Ala-Met-Tyr-OH, General Procedure 2

N-dodecanoyl-Ala-Ala-Met-Tyr-OH was prepared according to solid phase peptide synthesis—general procedure 2.

### Example 69

### N-dodecanoyl-Ala-Ala-Pro-Tyr-OH, General Procedure 2

N-dodecanoyl-Ala-Ala-Pro-Tyr-OH was prepared according to solid phase peptide synthesis—general procedure 2.

### Example 70

### N-dodecanoyl-Ala-Ala-Ser-Tyr-OH, General Procedure 2

N-dodecanoyl-Ala-Ala-Ser-Tyr-OH was prepared according to solid phase peptide synthesis—general procedure 2.

### Example 71

### N-dodecanoyl-Ala-Ala-Thr-Tyr-OH, General Procedure 2

N-dodecanoyl-Ala-Ala-Thr-Tyr-OH was prepared according to solid phase peptide synthesis—general procedure 2.

### Example 72

### N-dodecanoyl-Ala-Ala-Val-Tyr-OH, General Procedure 2

N-dodecanoyl-Ala-Ala-Val-Tyr-OH was prepared according to solid phase peptide synthesis—general procedure 2.

### Example 73

### N-dodecanoyl-Ala-Arg-Pro-Tyr-OH, General Procedure 2

N-dodecanoyl-Ala-Arg-Pro-Tyr-OH was prepared according to solid phase peptide synthesis—general procedure 2.

### Example 74

### N-dodecanoyl-Ala-Asn-Pro-Tyr-OH, General Procedure 2

N-dodecanoyl-Ala-Asn-Pro-Tyr-OH was prepared according to solid phase peptide synthesis—general procedure 2.

### Example 75

### N-dodecanoyl-Ala-Asp-Pro-Tyr-OH, General Procedure 2

N-dodecanoyl-Ala-Asp-Pro-Tyr-OH was prepared according to solid phase peptide synthesis—general procedure 2.

### Example 76

### N-dodecanoyl-Ala-Gln-Pro-Tyr-OH, General Procedure 2

N-dodecanoyl-Ala-Gln-Pro-Tyr-OH was prepared according to solid phase peptide synthesis—general procedure 2.

### Example 77

### N-dodecanoyl-Ala-Glu-Pro-Tyr-OH, General Procedure 2

N-dodecanoyl-Ala-Glu-Pro-Tyr-OH was prepared according to solid phase peptide synthesis—general procedure 2.

### Example 78N-dodecanoyl-Ala-Gly-Pro-Tyr-OH, General Procedure 2

N-dodecanoyl-Ala-Gly-Pro-Tyr-OH was prepared according to solid phase peptide synthesis—general procedure 2.

### Example 79

### N-dodecanoyl-Ala-His-Pro-Tyr-OH, General Procedure 2

N-dodecanoyl-Ala-His-Pro-Tyr-OH was prepared according to solid phase peptide synthesis—general procedure 2.

### Example 80

### N-dodecanoyl-Ala-Ile-Pro-Tyr-OH, General Procedure 2

N-dodecanoyl-Ala-Ile-Pro-Tyr-OH was prepared according to solid phase peptide synthesis—general procedure 2.

### Example 81

### N-dodecanoyl-Ala-Leu-Pro-Tyr-OH, General Procedure 2

N-dodecanoyl-Ala-Leu-Pro-Tyr-OH was prepared according to solid phase peptide synthesis—general procedure 2.

### Example 82

### N-dodecanoyl-Ala-Lys-Pro-Tyr-OH, General Procedure 2

N-dodecanoyl-Ala-Lys-Pro-Tyr-OH was prepared according to solid phase peptide synthesis—general procedure 2.

### Example 83

### N-dodecanoyl-Ala-Met-Pro-Tyr-OH, General Procedure 2

N-dodecanoyl-Ala-Met-Pro-Tyr-OH was prepared according to solid phase peptide synthesis—general procedure 2.

### Example 84

### N-dodecanoyl-Ala-Phe-Pro-Tyr-OH, General Procedure 2

N-dodecanoyl-Ala-Phe-Pro-Tyr-OH was prepared according to solid phase peptide synthesis—general procedure 2.

### Example 85

### N-dodecanoyl-Ala-Pro-Pro-Tyr-OH, General Procedure 2

N-dodecanoyl-Ala-Pro-Pro-Tyr-OH was prepared according to solid phase peptide synthesis—general procedure 2.

### Example 86

### N-dodecanoyl-Ala-Ser-Pro-Tyr-OH, General Procedure 2

N-dodecanoyl-Ala-Ser-Pro-Tyr-OH was prepared according to solid phase peptide synthesis—general procedure 2.

### Example 87

### N-dodecanoyl-Ala-Thr-Pro-Tyr-OH, General Procedure 2

N-dodecanoyl-Ala-Thr-Pro-Tyr-OH was prepared according to solid phase peptide synthesis—general procedure 2.

### Example 88

### N-dodecanoyl-Ala-Trp-Pro-Tyr-OH, General Procedure 2

N-dodecanoyl-Ala-Trp-Pro-Tyr-OH was prepared according to solid phase peptide synthesis—general procedure 2

### Example 89

### N-dodecanoyl-Ala-Tyr-Pro-Tyr-OH, General Procedure 2

N-dodecanoyl-Ala-Tyr-Pro-Tyr-OH was prepared according to solid phase peptide synthesis—general procedure 2.

### Example 90

### N-dodecanoyl-Ala-Val-Pro-Tyr-OH, General Procedure 2

N-dodecanoyl-Ala-Val-Pro-Tyr-OH was prepared according to solid phase peptide synthesis—general procedure 2.

### Example 91

### N-dodecanoyl-Arg-Ala-Ala-Pro-Tyr-OH, General Procedure 2

N-dodecanoyl-Arg-Ala-Ala-Pro-Tyr-OH was prepared according to solid phase peptide synthesis—general procedure 2.

### Example 92

### N-dodecanoyl-Arg-Ala-Pro-Tyr-OH, General Procedure 2

N-dodecanoyl-Arg-Ala-Pro-Tyr-OH was prepared according to solid phase peptide synthesis—general procedure 2.

### Example 93

### N-dodecanoyl-Asn-Ala-Ala-Pro-Tyr-OH, General Procedure 2

N-dodecanoyl-Asn-Ala-Ala-Pro-Tyr-OH was prepared according to solid phase peptide synthesis—general procedure 2.

### Example 94

### N-dodecanoyl-Asn-Ala-Pro-Tyr-OH, General Procedure 2

N-dodecanoyl-Asn-Ala-Pro-Tyr-OH was prepared according to solid phase peptide synthesis—general procedure 2.

### Example 95

### N-dodecanoyl-Asp-Ala-Ala-Pro-Tyr-OH, General Procedure 2

N-dodecanoyl-Asp-Ala-Ala-Pro-Tyr-OH was prepared according to solid phase peptide synthesis—general procedure 2.

### Example 96

### N-dodecanoyl-Asp-Ala-Pro-Tyr-OH, General Procedure 2

N-dodecanoyl-Asp-Ala-Pro-Tyr-OH was prepared according to solid phase peptide synthesis—general procedure 2.

### Example 97

### N-dodecanoyl-γGlu-Ala-Ala-Pro-Tyr-OH, General Procedure 2

N-dodecanoyl-γGlu-Ala-Ala-Pro-Tyr-OH was prepared according to solid phase peptide synthesis—general procedure 2.

### Example 98

### N-dodecanoyl-γGlu-Ala-Pro-Tyr-OH, General Procedure 2

N-dodecanoyl-γGlu-Ala-Pro-Tyr-OH was prepared according to solid phase peptide synthesis—general procedure 2.

### Example 99

### N-dodecanoyl-γGlu-γGlu-Ala-Ala-Pro-Tyr-OH, General Procedure 2

N-dodecanoyl-γGlu-γGlu-Ala-Ala-Pro-Tyr-OH was prepared according to solid phase peptide synthesis—general procedure 2.

### Example 100

### N-dodecanoyl-γGlu-Pro-Tyr-OH, General Procedure 2

N-dodecanoyl-γGlu-Pro-Tyr-OH was prepared according to solid phase peptide synthesis—general procedure 2.

### Example 101

### N-dodecanoyl-γGlu-Tyr-OH, General Procedure 2

N-dodecanoyl-γGlu-Tyr-OH was prepared according to solid phase peptide synthesis—general procedure 2.

### Example 102

### N-dodecanoyl-Gln-Ala-Ala-Pro-Tyr-OH, General Procedure 2

N-dodecanoyl-Gln-Ala-Ala-Pro-Tyr-OH was prepared according to solid phase peptide synthesis—general procedure 2.

### Example 103

### N-dodecanoyl-Gln-Ala-Pro-Tyr-OH, General Procedure 2

N-dodecanoyl-Gln-Ala-Pro-Tyr-OH was prepared according to solid phase peptide synthesis—general procedure 2.

### Example 104

### N-dodecanoyl-Glu-Ala-Ala-Pro-Tyr-OH, General Procedure 2

N-dodecanoyl-Glu-Ala-Ala-Pro-Tyr-OH was prepared according to solid phase peptide synthesis—general procedure 2.

### Example 105

### N-dodecanoyl-Glu-Ala-Pro-Tyr-OH, General Procedure 2

N-dodecanoyl-Glu-Ala-Pro-Tyr-OH was prepared according to solid phase peptide synthesis—general procedure 2.

### Example 106

### N-dodecanoyl-Pro-Pro-Tyr-OH, General Procedure 2

N-dodecanoyl-Pro-Pro-Tyr-OH was prepared according to solid phase peptide synthesis—general procedure 2.

### Example 107

### N-dodecanoyl-Ser-Pro-Tyr-OH, General Procedure 2

N-dodecanoyl-Ser-Pro-Tyr-OH was prepared according to solid phase peptide synthesis—general procedure 2.

### Example 108

### N-dodecanoyl-Thr-Pro-Tyr-OH, General Procedure 2

N-dodecanoyl-Thr-Pro-Tyr-OH was prepared according to solid phase peptide synthesis—general procedure 2.

### Example 109

### N-dodecanoyl-Trp-Pro-Tyr-OH, General Procedure 2

N-dodecanoyl-Trp-Pro-Tyr-OH was prepared according to solid phase peptide synthesis—general procedure 2.

### Example 110

### N-dodecanoyl-Tyr-Pro-Tyr-OH, General Procedure 2

N-dodecanoyl-Tyr-Pro-Tyr-OH was prepared according to solid phase peptide synthesis—general procedure 2.

### Example 111

### N-dodecanoyl-Val-Pro-Tyr-OH, General Procedure 2

N-dodecanoyl-Val-Pro-Tyr-OH was prepared according to solid phase peptide synthesis—general procedure 2.

### Example 112

### N-dodecanoyl-Ala-Val-Tyr-OH, General Procedure 2

N-dodecanoyl-Ala-Val-Tyr-OH was prepared according to solid phase peptide synthesis—general procedure 2.

### Example 113

### N-dodecanoyl-Arg-Pro-Tyr-OH, General Procedure 2

N-dodecanoyl-Arg-Pro-Tyr-OH was prepared according to solid phase peptide synthesis—general procedure 2.

### Example 114

### N-dodecanoyl-Asn-Pro-Tyr-OH, General Procedure 2

N-dodecanoyl-Asn-Pro-Tyr-OH was prepared according to solid phase peptide synthesis—general procedure 2.

### Example 115

### N-dodecanoyl-Asp-Pro-Tyr-OH, General Procedure 2

N-dodecanoyl-Asp-Pro-Tyr-OH was prepared according to solid phase peptide synthesis—general procedure 2.

### Example 116

### N-dodecanoyl-Gln-Pro-Tyr-OH, General Procedure 2

N-dodecanoyl-Gln-Pro-Tyr-OH was prepared according to solid phase peptide synthesis—general procedure 2.

### Example 117

### N-dodecanoyl-Glu-Pro-Tyr-OH, General Procedure 2

N-dodecanoyl-Glu-Pro-Tyr-OH was prepared according to solid phase peptide synthesis—general procedure 2.

### Example 118

### N-dodecanoyl-Gly-Pro-Tyr-OH, General Procedure 2

N-dodecanoyl-Gly-Pro-Tyr-OH was prepared according to solid phase peptide synthesis—general procedure 2.

### Example 119

### N-dodecanoyl-His-Pro-Tyr-OH, General Procedure 2

N-dodecanoyl-His-Pro-Tyr-OH was prepared according to solid phase peptide synthesis—general procedure 2.

### Example 120

### N-dodecanoyl-Ile-Pro-Tyr-OH, General Procedure 2

N-dodecanoyl-Ile-Pro-Tyr-OH was prepared according to solid phase peptide synthesis—general procedure 2.

### Example 121

### N-dodecanoyl-Leu-Pro-Tyr-OH, General Procedure 2

N-dodecanoyl-Leu-Pro-Tyr-OH was prepared according to solid phase peptide synthesis—general procedure 2.

### Example 122

### N-dodecanoyl-Lys-Pro-Tyr-OH, General Procedure 2

N-dodecanoyl-Lys-Pro-Tyr-OH was prepared according to solid phase peptide synthesis—general procedure 2.

### Example 123

### N-dodecanoyl-Met-Pro-Tyr-OH, General Procedure 2

N-dodecanoyl-Met-Pro-Tyr-OH was prepared according to solid phase peptide synthesis—general procedure 2.

### Example 124

### N-dodecanoyl-Phe-Pro-Tyr-OH, General Procedure 2

N-dodecanoyl-Phe-Pro-Tyr-OH was prepared according to solid phase peptide synthesis—general procedure 2.

### Example 125

### N-dodecanoyl-γGlu-OEG-Ala-Ala-Pro-Phe-OH, General Procedure 1

Alternative name: (S)-4-(2-{2-[((S)-1-{(S)-2-[(S)-2-((S)-1-Carboxy-2-phenyl-ethylcarbamoyl)-pyrrolidin-1-yl]-1-methyl-2-oxo-ethylcarbamoyl}-ethylcarbamoyl)-methoxy]-ethoxy}-ethylcarbamoyl)-2-dodecanoylamino-butyric acid.

N-dodecanoyl-γGlu-OEG-Ala-Ala-Pro-Phe-OH was prepared according to solid phase peptide synthesis—general procedure 1.

1H-NMR (300 MHz, AcOD-d4, 80° C., dH): 7.37-7.18 (m, 5H); 4.90 (t, J=6.2 Hz, 1H); 4.80 (m, 1H); 4.70-4.55 (m, 3H); 4.12 (s, 2H); 3.87-3.39 (m, 10H); 3.20 (m, 2H); 2.44 (t, J=6.2 Hz, 2H); 2.34 (t, J=7.5 Hz, 2H); 2.30-1.91 (m, 6H); 1.67 (m, 2H); 1.42 (d, J=7.0 Hz, 3H); 1.41-1.25 (m, 19H); 0.91 (t, J=6.5 Hz, 3H).

LC-MS purity: 95% (ELSD).

LC-MS Rt (Sunfire 4.6 mm×100 mm, acetonitrile/water 35:65 to 100:0+0.1% FA): 6.03 min.

LC-MS m/z: 861.6 (M+H).

TLC: RF (SiO2, dichloromethane/methanol 4:1): 0.10.

### Example 126

### N-tetradecanoyl-γGlu-OEG-Ala-Ala-Pro-Phe-OH, General Procedure 1

Alternative name: (S)-4-(2-{2-[((S)-1-{(S)-2-[(S)-2-((S)-1-Carboxy-2-phenyl-ethylcarbamoyl)-pyrrolidin-1-yl]-1-methyl-2-oxo-ethylcarbamoyl}-ethylcarbamoyl)-methoxy]-ethoxy}-ethylcarbamoyl)-2-tetradecanoylamino-butyric acid.

N-tetradecanoyl-γGlu-OEG-Ala-Ala-Pro-Phe-OH was prepared according to solid phase peptide synthesis—general procedure 1.

1H-NMR (300 MHz, AcOD-d4, 80° C., H): 7.38-7.19 (m, 5H); 4.90 (t, J=6.3 Hz, 1H); 4.80 (m, 1H); 4.71-4.55 (m, 3H); 4.12 (s, 2H); 3.86-3.42 (m, 10H); 3.20 (m, 2H); 2.45 (t, J=6.6 Hz, 2H); 2.34 (t, J=7.6 Hz, 2H); 2.30-1.93 (m, 6H); 1.66 (m, 2H); 1.42 (d, J=7.0 Hz, 3H); 1.41-1.24 (m, 23H); 0.91 (t, J=6.5 Hz, 3H).

LC-MS purity: 96% (ELSD).

LC-MS Rt (Sunfire 4.6 mm×100 mm, acetonitrile/water 35:65 to 100:0+0.1% FA): 6.80 min.

LC-MS m/z: 889.7 (M+H).

TLC: RF (SiO2, dichloromethane/methanol 4:1): 0.10.

### Example 127

### N-dodecanoyl-γGlu-OEG-Pro-Arg-OH, General Procedure 1

Alternative name: (S)-2-{[(S)-1-(2-{2-[2-((S)-4-Carboxy-4-dodecanoylamino-butyrylamino)-ethoxy]-ethoxy}-acetyl)-pyrrolidine-2-carbonyl]-amino}-5-guanidino-pentanoic acid.

N-dodecanoyl-γGlu-OEG-Pro-Arg-OH was prepared according to solid phase peptide synthesis—general procedure 1.

1H-NMR (300 MHz, AcOD-d4, 80° C., dH): 4.73-4.52 (m, 3H); 4.31 (s, 3H); 3.81-3.39 (m, 10H); 3.31 (t, J=6.1 Hz, 2H); 2.45 (t, J=6.9 Hz, 2H); 2.35 (t, J=7.6 Hz, 2H); 2.29-1.58 (m, 12H); 1.32 (bs, 16H); 0.91 (t, J=6.0 Hz, 3H).

LC-MS purity: 100% (ELSD).

LC-MS Rt (Sunfire 4.6 mm×100 mm, acetonitrile/water 20:80 to 100:0+0.1% FA): 5.13 min.

LC-MS m/z: 728.6 (M+H).

TLC: RF (SiO2, dichloromethane/methanol 4:1): 0.10.

### Example 128

### N-dodecanoyl-OEG-OEG-Phe-OH, General Procedure 1

Alternative name: (S)-2-{2-[2-(2-{2-[2-(2-Dodecanoylamino-ethoxy)-ethoxy]-acetylamino}-ethoxy)-ethoxy]-acetylamino}-3-phenyl-propionic acid.

N-dodecanoyl-OEG-OEG-Phe-OH was prepared according to solid phase peptide synthesis—general procedure 1.

1H-NMR (300 MHz, CDCl3, H): 7.56 (d, J=8.1 Hz, 1H); 7.37 (t, J=5.7 Hz, 1H); 7.34-7.15 (m, 5H); 6.49 (t, J=5.3 Hz, 1H); 4.97 (m, 1H); 4.04 (s, 2H); 4.01 (s, 2H); 3.71-3.03 (m, 18H); 2.21 (t, J=7.7 Hz, 2H); 1.61 (m, 2H); 1.26 (bs, 16H); 0.88 (t, J=6.7 Hz, 3H).

LC-MS purity: 97% (ELSD).

LC-MS Rt (Sunfire 4.6 mm×100 mm, acetonitrile/water 20:80 to 100:0+0.1% FA): 7.81 min.

LC-MS m/z: 638.5 (M+H).

TLC: RF (SiO2, dichloromethane/methanol 4:1): 0.10.

### Example 129

### N-dodecanoyl-OEG-OEG-DPhe-OH, General Procedure 1

Alternative name: (R)-2-{2-[2-(2-{2-[2-(2-Dodecanoylamino-ethoxy)-ethoxy]-acetylamino}-ethoxy)-ethoxy]-acetylamino}-3-phenyl-propionic acid.

N-dodecanoyl-OEG-OEG-DPhe-OH was prepared according to solid phase peptide synthesis—general procedure 1.

1H-NMR (300 MHz, CDCl3, dH): 7.47 (d, J=8.1 Hz, 1H); 7.31-7.14 (m, 6H); 6.39 (t, J=5.2 Hz, 1H); 4.96 (m, 1H); 4.00 (s, 2H); 3.98 (s, 2H); 3.70-3.05 (m, 18H); 2.19 (t, J=7.6 Hz, 2H); 1.61 (m, 2H); 1.26 (bs, 16H); 0.88 (t, J=6.7 Hz, 3H).

LC-MS purity: 100% (ELSD).

LC-MS Rt (Sunfire 4.6 mm×100 mm, acetonitrile/water 20:80 to 100:0+0.1% FA): 8.13 min.

LC-MS m/z: 638.5 (M+H).

TLC: RF (SiO2, dichloromethane/methanol 4:1): 0.10.

### Example 130

### N-dodecanoyl-OEG-OEG-Phe-OEG-OH, General Procedure 1

Alternative name: {2-[2-((S)-2-{2-[2-(2-{2-[2-(2-Dodecanoylamino-ethoxy)-ethoxy]-acetylamino}-ethoxy)-ethoxy]-acetylamino}-3-phenyl-propionylamino)-ethoxy]-ethoxy}-acetic acid.

N-dodecanoyl-OEG-OEG-Phe-OEG-OH was prepared according to solid phase peptide synthesis—general procedure 1.

1H-NMR (300 MHz, CDCl3, dH): 7.54 (d, J=8.7 Hz, 1H); 7.42 (t, J=4.8 Hz, 1H); 7.33-7.17 (m, 5H); 7.00 (t, J=5.1 Hz, 1H); 6.37 (t, J=5.3 Hz, 1H); 4.89 (q, J=7.8 Hz, 1H); 4.13 (s, 2H); 4.04 (s, 2H); 3.92 (m, 2H); 3.74-3.22 (m, 24H); 3.07 (m, 2H); 2.20 (t, J=7.6 Hz, 2H); 1.61 (m, 2H); 1.25 (bs, 16H); 0.88 (t, J=6.7 Hz, 3H).

LC-MS purity: 100% (ELSD).

LC-MS Rt (Sunfire 4.6 mm×100 mm, acetonitrile/water 20:80 to 100:0+0.1% FA): 7.47 min.

LC-MS m/z: 783.6 (M+H).

TLC: RF (SiO2, dichloromethane/methanol 4:1): 0.10.

### Example 131

### N-dodecanoyl-OEG-OEG-DPhe-OEG-OH, General Procedure 1

Alternative name: {2-[2-((R)-2-{2-[2-(2-{2-[2-(2-Dodecanoylamino-ethoxy)-ethoxy]-acetylamino}-ethoxy)-ethoxy]-acetylamino}-3-phenyl-propionylamino)-ethoxy]-ethoxy}-acetic acid.

N-dodecanoyl-OEG-OEG-DPhe-OEG-OH was prepared according to solid phase peptide synthesis—general procedure 1.

1H-NMR (300 MHz, CDCl3, dH): 7.53 (d, J=8.3 Hz, 1H); 7.40 (bs, 1H); 7.33-7.17 (m, 5H); 6.98 (bs, 1H); 6.34 (bs, 1H); 4.90 (q, J=7.7 Hz, 1H); 4.14 (s, 2H); 4.04 (s, 2H); 3.93 (m, 2H); 3.75-3.21 (m, 24H); 3.08 (m, 2H); 2.20 (t, J=7.6 Hz, 2H); 1.62 (m, 2H); 1.25 (bs, 16H); 0.88 (t, J=6.6 Hz, 3H).

LC-MS purity: 100% (ELSD).

LC-MS Rt (Sunfire 4.6 mm×100 mm, acetonitrile/water 20:80 to 100:0+0.1% FA): 7.46 min.

LC-MS m/z: 783.6 (M+H).

TLC: RF (SiO2, dichloromethane/methanol 4:1): 0.10.

### Example 132

### N-dodecanoyl-γGlu-OEG-OEG-Arg-OH, General Procedure 1

Alternative name: (S)-2-(2-{2-[2-(2-{2-[2-((S)-4-Carboxy-4-dodecanoylamino-butyrylamino)-ethoxy]-ethoxy}-acetylamino)-ethoxy]-ethoxy}-acetylamino)-5-guanidino-pentanoic acid.

N-dodecanoyl-γGlu-OEG-OEG-Arg-OH was prepared according to solid phase peptide synthesis—general procedure 1.

1H-NMR (300 MHz, AcOD-d4, 80° C., dH): 4.69 (t, J=6.0 Hz, 1H); 4.60 (t, J=6.4 Hz, 1H); 4.16 (s, 2H); 4.11 (s, 2H); 3.82-3.61 (m, 12H); 3.58-3.43 (m, 4H); 3.33 (t, J=6.1 Hz, 2H); 2.45 (t, J=7.0 Hz, 2H); 2.34 (t, J=7.4 Hz, 2H); 2.31-1.84 (m, 4H); 1.80 (m, 2H); 1.66 (m, 2H); 1.32 (bs, 16H); 0.90 (t, J=5.9 Hz, 3H).

LC-MS purity: 100% (ELSD).

LC-MS Rt (Sunfire 4.6 mm×100 mm, acetonitrile/water 20:80 to 100:0+0.1% FA): 5.07 min.

LC-MS m/z: 776.6 (M+H).

TLC: RF (SiO2, dichloromethane/methanol 4:1): 0.10.

### Example 133

### N-dodecanoyl-γGlu-OEG-OEG-DArg-OH, General Procedure 1

Alternative name: (R)-2-(2-{2-[2-(2-{2-[2-((S)-4-Carboxy-4-dodecanoylamino-butyrylamino)-ethoxy]-ethoxy}-acetylamino)-ethoxy]-ethoxy}-acetylamino)-5-guanidino-pentanoic acid.

N-dodecanoyl-γGlu-OEG-OEG-DArg-OH was prepared according to solid phase peptide synthesis—general procedure 1.

1H-NMR (300 MHz, AcOD-d4, 80° C., dH): 4.70 (dd, J=7.6 and 5.4 Hz, 1H); 4.61 (dd, J=8.0 and 5.2 Hz, 1H); 4.16 (s, 2H); 4.12 (s, 2H); 3.82-3.61 (m, 12H); 3.59-3.44 (m, 4H); 3.33 (td, J=6.7 and 1.6 Hz, 2H); 2.45 (t, J=7.0 Hz, 2H); 2.35 (t, 6.7 Hz, 3H).

LC-MS purity: 100% (ELSD).

LC-MS Rt (Sunfire 4.6 mm×100 mm, acetonitrile/water 20:80 to 100:0+0.1% FA): 5.04 min.

LC-MS m/z: 776.7 (M+H).

TLC: RF (SiO2, dichloromethane/methanol 4:1): 0.10.

### Example 134

### N-hexadecanoyl-γGlu-OEG-Ala-Ala-Pro-Phe-OH, General Procedure 1

Alternative name: (S)-4-(2-{2-[((S)-1-{(S)-2-[(S)-2-((S)-1-Carboxy-2-phenyl-ethylcarbamoyl)-pyrrolidin-1-yl]-1-methyl-2-oxo-ethylcarbamoyl}-ethylcarbamoyl)-methoxy]-ethoxy}-ethylcarbamoyl)-2-hexadecanoylaminobutyric acid.

N-hexadecanoyl-γGlu-OEG-Ala-Ala-Pro-Phe-OH was prepared according to solid phase peptide synthesis—general procedure 1.

1H-NMR (300 MHz, AcOD-d4, 80° C., dH): 7.35-7.18 (m, 5H); 4.97-4.52 (m, 5H); 4.12 (s 2H); 3.87-3.42 (m, 10H); 3.31-3.08 (m, 2H); 2.51-1.98 (m, 10H); 1.74-1.61 (m, 2H); 1.46-1.27 (m, 30H); 0.95-0.85 (m, 3H).

LC-MS purity: 98% (ELSD).

LC-MS Rt (Sunfire 4.6 mm×100 mm, acetonitrile/water 70:30 to 100:0+0.1% FA): 4.85 min.

LC-MS m/z: 917.8 (M+H).

### Example 135

### N-hexadecanoyl-γGlu-Ala-Ala-Pro-Phe-OHGeneral procedure 1

Alternative name: (S)-4-((S)-1-{(S)-2-[(S)-2-((S)-1-Carboxy-2-phenyl-ethylcarbamoyl)-pyrrolidin-1-yl]-1-methyl-2-oxo-ethylcarbamoyl}-ethylcarbamoyl)-4-hexadecanoylamino-butyric acid.

N-hexadecanoyl-γGlu-Ala-Ala-Pro-Phe-OH was prepared according to solid phase peptide synthesis—general procedure 1.

1H-NMR (300 MHz, AcOD-d4, 80° C., dH): 7.34-7.18 (m, 5H); 4.98-4.41 (m, 5H); 3.88-3.43 (m, 2H); 3.35-3.05 (m, 2H); 2.48 (t, J=7.5 Hz, 2H); 2.32 (t, J=7.5 Hz, 2H); 2.23-1.90 (m, 6H); 1.72-1.55 (m, 2H); 1.46-1.17 (m, 30H); 0.96-0.84 (m, 3H).

LC-MS purity: 98% (ELSD).

LC-MS Rt (Sunfire 4.6 mm×100 mm, acetonitrile/water 70:30 to 100:0+0.1% FA): 4.55 min. LC-MS m/z: 772.5 (M+H).

### Example 136

### N-tetradecanoyl-βAla-βAla-Pro-Phe-OHGeneral procedure 1

Alternative name: (S)-3-Phenyl-2-({(S)-1-[3-(3-tetradecanoylamino-propionylamino)-propionyl]-pyrrolidine-2-carbonyl}-amino)-propionic acid.

N-tetradecanoyl-βAla-βAla-Pro-Phe-OH was prepared according to solid phase peptide synthesis—general procedure 1.

1H-NMR (300 MHz, AcOD-d4, 80° C., dH): 7.37-7.17 (m, 5H); 5.11-4.41 (m, 2H); 3.63-2.97 (m, 8H); 2.69-1.70 (m, 10H); 1.70-1.56 (m, 2H); 1.32 (s, 20H); 0.99-0.83 (m, 3H).

LC-MS purity: 100% (ELSD).

LC-MS Rt (Sunfire 4.6 mm×100 mm, acetonitrile/water 70:30 to 100:0+0.1% FA): 4.83 min.

LC-MS m/z: 615.4 (M+H).

### Example 137

### N-tetradecanoyl-βAla-βAla-βAla-βAla-Pro-Phe-OHGeneral procedure 1

Alternative name: (S)-3-Phenyl-2-{[(S)-1-(3-{3-[3-(3-tetradecanoylamino-propionylamino)-propionylamino]-propionylamino}-propionyl)-pyrrolidine-2-carbonyl]-amino}-propionic acid.

N-tetradecanoyl-βAla-βAla-βAla-βAla-Pro-Phe-OH was prepared according to solid phase peptide synthesis—general procedure 1.

1H-NMR (300 MHz, AcOD-d4, 80° C., dH): 7.37-7.17 (m, 5H); 5.08-4.39 (m, 2H); 3.66-3.00 (m, 12H); 2.73-1.71 (m, 14H); 1.69-1.54 (m, 2H); 1.32 (s, 20H); 1.00-0.81 (m, 3H).

LC-MS purity: 98% (ELSD).

LC-MS Rt (Sunfire 4.6 mm×100 mm, acetonitrile/water 70:30 to 100:0+0.1% FA): 2.90 min. LC-MS m/z: 757.5 (M+H).

### Example 138

### N-tetradecanoyl-βAla-βAla-βAla-Pro-Phe-OHGeneral procedure 1

Alternative name: (S)-3-phenyl-2-[((S)-1-{3-[3-(3-tetradecanoylamino-propionylamino)-propionylamino]-propionyl}-pyrrolidine-2-carbonyl)-amino]-propionic acid.

N-tetradecanoyl-βAla-βAla-βAla-Pro-Phe-OH was prepared according to solid phase peptide synthesis—general procedure 1.

1H-NMR (300 MHz, AcOD-d4, 80° C., dH): 7.35-7.18 (m, 5H); 5.07-4.40 (m, 2H); 3.63-3.03 (m, 10H); 2.69-1.68 (m, 12H); 1.69-1.54 (m, 2H); 1.32 (s, 20H); 0.98-0.83 (m, 3H).

LC-MS purity: 100% (ELSD).

LC-MS Rt (Sunfire 4.6 mm×100 mm, acetonitrile/water 70:30 to 100:0+0.1% FA): 3.62 min.

LC-MS m/z: 686.4 (M+H).

### Example 139

### N-tetradecanoyl-γGlu-βAla-βAla-Pro-Phe-OH General Procedure 1

Alternative name: (S)-4-[2-(2-{3-[(S)-2-((S)-1-Carboxy-2-phenyl-ethylcarbamoyl)-pyrrolidin-1-yl]-3-oxo-propylcarbamoyl}-ethylcarbamoyl)-ethylcarbamoyl]-2-tetradecanoylamino-butyric acid.

N-tetradecanoyl-γGlu-βAla-βAla-Pro-Phe-OH was prepared according to solid phase peptide synthesis—general procedure 1.

1H-NMR (300 MHz, AcOD-d4, 80° C.dH): 7.39-7.14 (m, 5H); 5.08-4.36 (m, 3H); 3.68-2.97 (m, 10H); 2.73-1.74 (m, 16H); 1.72-1.58 (m, 2H); 1.32 (s, 20H); 0.97-0.84 (m, 3H).

LC-MS purity: 100% (ELSD).

LC-MS Rt (Sunfire 4.6 mm×100 mm, acetonitrile/water 70:30 to 100:0+0.1% FA): 2.44 min.

LC-MS m/z: 815.5 (M+H).

### Example 140

### N-tetradecanoyl-Ala-Ala-Ala-Ala-Pro-Phe-OH, General Procedure 1

Alternative name: (S)-3-Phenyl-2-{[(S)-1-((S)-2-{(S)-2-[(S)-2-((S)-2-tetradecanoylamino-propionylamino)-propionylamino]-propionylamino}-propionyl)-pyrrolidine-2-carbonyl]-amino}-propionic acid

N-tetradecanoyl-Ala-Ala-Ala-Ala-Pro-Phe-OH was prepared according to solid phase peptide synthesis—general procedure 1

1H-NMR (300 MHz, AcOD, 80° C., dH): 7.39-7.15 (m, 5H); 4.99-4.45 (m, 6H); 3.93-3.46 (m, 2H); 3.42-3.05 (m, 2H); 2.31 (t, J=7.4 Hz, 2H); 2.21-1.91 (m, 4H); 1.76-1.55 (m, 2H); 1.50-1.19 (m, 32H); 1.01-0.83 (m, 3H).

LC-MS purity: 98% (ELSD)

LC-MS Rt (Sunfire 4.6 mm×100 mm, acetonitrile/water 70:30 to 100:0+0.1% FA): 4.32 min

LC-MS m/z: 757.5 (M+H).

### Example 141

### N-dodecanoyl-Ala-Ala-Ala-Ala-Ala-Pro-Phe-OH, General Procedure 1

Alternative name: (S)-2-({(S)-1-[(S)-2-((S)-2-{(S)-2-[(S)-2-((S)-2-Dodecanoylamino-propionylamino)-propionylamino]-propionylamino}-propionylamino)-propionyl]-pyrrolidine-2-carbonyl}-amino)-3-phenyl-propionic acid

N-dodecanoyl-Ala-Ala-Ala-Ala-Ala-Pro-Phe-OH was prepared according to solid phase peptide synthesis—general procedure 1

1H-NMR (300 MHz, AcOD, 80° C., dH): 7.35-7.19 (m, 5H); 4.98-4.42 (m, 7H); 3.89-3.43 (m, 2H); 3.39-3.06 (m, 2H); 2.37-1.89 (m, 6H); 1.70-1.53 (m, 2H); 1.51-1.06 (m, 31H); 0.91 (t, J=6.4 Hz, 3H).

LC-MS purity: 98% (ELSD).

LC-MS Rt (Sunfire 4.6 mm×100 mm, acetonitrile/water 50:50 to 100:0+0.1% FA): 4.94 min.

LC-MS m/z: 800.5 (M+H).

### Example 142

### N-tetradecanoyl-Leu-Ala-Ala-Pro-Tyr-OH, General Procedure 1

Alternative name: N{1}-tetradecanoyl-Leu-Ala-Ala-Pro-Tyr

N-tetradecanoyl-Leu-Ala-Ala-Pro-Tyr-OH was prepared according to solid phase peptide synthesis—general procedure 1

1H-NMR (300 MHz, AcOD-d4, 80° C., dH): 7.15-7.01 (m, 2H); 6.82-6.73 (m, 2H); 4.90-4.72 (m, 2H); 4.71-4.46 (m, 3H); 3.85-3.69 (m, 1H); 3.69-3.54 (m, 1H); 3.25-2.94 (m, 2H); 2.36-2.21 (m, 2H); 2.19-1.92 (m, 4H); 1.75-1.51 (m, 5H); 1.46-1.19 (m, 26H); 0.99-0.82 (m, 9H).

LC-MS purity: 100% (ELSD).

LC-MS Rt (Sunfire 4.6 mm×100 mm, acetonitrile/water 70:30 to 100:0+0.1% FA): 4.78 min.

LC-MS m/z: 743.5 (M+H).

### Example 143

### N-tetradecanoyl-Glu-Ala-Ala-Pro-Trp-OH, General Procedure 1

Alternative name: N{1}-tetradecanoyl-Glu-Ala-Ala-Pro-Trp-OH N-tetradecanoyl-Glu-Ala-Ala-Pro-Trp-OH was prepared according to solid phase peptide synthesis—general procedure 1

1H-NMR (300 MHz, AcOD-d4, 80° C., dH): 7.61 (d, J=7.9 Hz, 1H); 7.36 (d, J=7.5 Hz, 1H); 7.20-7.00 (m, 3H); 4.96 (t, J=6.2 Hz, 1H); 4.80-4.47 (m, 4H); 3.82-3.65 (m, 1H); 3.59-3.45 (m, 1H); 3.44-3.30 (m, 2H); 2.48 (t, J=7.8 Hz, 2H); 2.32 (t, J=7.5 Hz, 2H); 2.22-1.91 (m, 6H); 1.72-1.57 (m, 2H); 1.49-1.16 (m, 26H); 0.95-0.85 (m, 3H).

LC-MS purity: 100% (ELSD).

LC-MS Rt (Sunfire 4.6 mm×100 mm, acetonitrile/water 70:30 to 100:0+0.1% FA): 3.58 min.

LC-MS m/z: 782.4 (M+H).

### Example 144

### N-hexadecanoyl-Glu-Ala-Ala-Pro-D-Phe-OH, General Procedure 1

Alternative name: N{1}-hexadecanoyl-Glu-Ala-Ala-Pro-D-Phe-OH

N-hexadecanoyl-Glu-Ala-Ala-Pro-D-Phe-OH was prepared according to solid phase peptide synthesis—general procedure 1

1H-NMR (300 MHz, AcOD-d4, 80° C., dH): 7.35-7.16 (m, 5H); 4.98-4.85 (m, 1H); 4.85-4.71 (m, 1H); 4.72-4.47 (m, 3H); 3.84-3.69 (m, 1H); 3.68-3.53 (m, 1H); 3.35-3.19 (m, 1H); 3.13-2.99 (m, 1H); 2.48 (t, J=7.4 Hz, 2H); 2.31 (t, J=7.4 Hz, 2H); 2.23-1.76 (m, 6H); 1.72-1.55 (m, 2H); 1.42-1.21 (m, 30H); 0.96-0.84 (m, 3H).

LC-MS purity: 98% (ELSD).

LC-MS Rt (Sunfire 4.6 mm×100 mm, acetonitrile/water 70:30 to 100:0+0.1% FA): 5.45 min.

LC-MS m/z: 794.5 (M+Na)+.

### Example 145

### N-tetradecanoyl-Leu-betaAla-Ala-Pro-DPhe-OH, General Procedure 1

Alternative name: N{1}-[(2R)-5-[[(2S)-4-methyl-2-(tetradecanoylamino)pentanoyl]amino]-3-oxopentan-2-yl]carbamoyl-Pro-D-Phe-OH

N-tetradecanoyl-Leu-betaAla-Ala-Pro-DPhe-OH was prepared according to solid phase peptide synthesis—general procedure 1

1H-NMR (300 MHz, AcOD-d4, 80° C., dH): 7.34-7.15 (m, 5H); 4.99-4.84 (m, 1H); 4.83-4.70 (m, 1H); 4.65-4.51 (m, 2H); 3.86-3.72 (m, 1H); 3.69-3.57 (m, 1H); 3.57-3.47 (m, 2H); 3.34-3.20 (m, 1H); 3.13-2.98 (m, 1H); 2.58-2.46 (m, 2H); 2.35-2.22 (m, 2H); 2.10-1.85 (m, 4H); 1.73-1.53 (m, 5H); 1.42-1.21 (m, 23H); 0.99-0.82 (m, 9H).

LC-MS purity: 97% (ELSD).

LC-MS Rt (Sunfire 4.6 mm×100 mm, acetonitrile/water 70:30 to 100:0+0.1% FA): 5.73 min.

LC-MS m/z: 727.5 (M+H).

### Example 146

### N-tetradecanoyl-Arg-Pro-Leu-bAla-Ala-Pro-D-Phe-OH, General procedure 1

Alternative name: N{Alpha-1}-tetradecanoyl-Arg-Pro-Leu-bAla-Ala-Pro-D-Phe-OHN-tetradecanoyl-Arg-Pro-Leu-bAla-Ala-Pro-D-Phe-OH was prepared according to solid phase peptide synthesis—general procedure 1

1H-NMR (300 MHz, AcOD-d4, 80° C., dH): 7.35-7.16 (m, 5H); 4.98-4.84 (m, 2H); 4.83-4.67 (m, 1H); 4.65-4.42 (m, 3H); 3.97-3.83 (m, 1H); 3.83-3.68 (m, 2H); 3.68-3.45 (m, 3H); 3.34-3.21 (m, 3H); 3.13-2.99 (m, 1H); 2.59-2.47 (m, 2H); 2.36-2.26 (m, 2H); 2.27-1.85 (m, 8H); 1.85-1.50 (m, 9H); 1.47-1.15 (m, 23H); 0.99-0.85 (m, 9H).

LC-MS purity: 97% (ELSD).

LC-MS Rt (Sunfire 4.6 mm×100 mm, acetonitrile/water 50:50 to 100:0+0.1% FA): 2.02 min.

LC-MS m/z: 980.6 (M+H).

### Example 147

### N-hexadecanoyl-Ala-Ala-Pro-D-Phe-OH, General Procedure 1

Alternative name: N{1}-hexadecanoyl-Ala-Ala-Pro-D-Phe-OH

N-hexadecanoyl-Ala-Ala-Pro-D-Phe-OH was prepared according to solid phase peptide synthesis—general procedure 1

1H-NMR (300 MHz, AcOD-d4, 80° C., dH): 7.34-7.18 (m, 5H); 4.92 (dd, J=7.9 and 5.3 Hz, 1H); 4.82-4.69 (m, 1H); 4.67-4.54 (m, 2H); 3.85-3.69 (m, 1H); 3.69-3.54 (m, 1H); 3.34-3.22 (m, 1H); 3.14-2.98 (m, 1H); 2.29 (t, J=7.6 Hz, 2H); 2.11-1.79 (m, 4H); 1.72-1.55 (m, 2H); 1.41-1.23 (m, 30H); 0.97-0.85 (m, 3H).

LC-MS purity: 100% (ELSD).

LC-MS Rt (Sunfire 4.6 mm×100 mm, acetonitrile/water 70:30 to 100:0+0.1% FA): 7.11 min.

LC-MS m/z: 642.3 (M+H)+

### Example 148

### N-tetradecanoyl-γGlu-DAla-DPro-DPhe-OH, General Procedure 1

Alternative name: N{1}-[(4S)-4-carboxy-4-(tetradecanoylamino)butanoyl]-D-Ala-D-Pro-D-Phe-OH

N-tetradecanoyl-γGlu-DAla-DPro-DPhe-OH was prepared according to solid phase peptide synthesis—general procedure 1

1H-NMR (300 MHz, AcOD-d4, 80° C.): 7.39-7.01 (m, 5H), 5.00-4.43 (m, 4H), 3.93-3.01 (m, 4H), 2.55-2.06 (m, 10H), 1.82-1.57 (m, 2H), 1.51-1.20 (m, 23H), 1.02-0.74 (m, 3H).

LC-MS purity: 100%.

LC-MS Rt (Sunfire 4.6 mm×100 mm, acetonitrile/water 70:30 to 100:0+0.1% FA): 4.70 min.

LC-MS m/z: 673.9 (M+H)⁺.

### Example 149

### N-hexadecanoyl-γGlu-Ala-Pro-Phe-OH, General Procedure 1

Alternative name: (S)-4-{(S)-2-[(S)-2-((S)-1-Carboxy-2-phenyl-ethylcarbamoyl)-pyrrolidin-1-yl]-1-methyl-2-oxo-ethylcarbamoyl}-2-hexadecanoylamino-butyric acid

N-hexadecanoyl-γGlu-Ala-Pro-Phe-OH was prepared according to solid phase peptide synthesis—general procedure 1

1H-NMR (300 MHz, AcOD-d4, 80° C., dH): 7.36-7.16 (m, 5H); 5.01-4.43 (m, 4H); 3.93-3.40 (m, 2H); 3.33-3.05 (m, 2H); 2.54-1.89 (m, 10H); 1.77-1.58 (m, 2H); 1.47-1.18 (m, 27H); 0.99-0.82 (m, 3H).

LC-MS purity: 100% (ELSD).

LC-MS Rt (Sunfire 4.6 mm×100 mm, acetonitrile/water 70:30 to 100:0+0.1% FA): 6.25 min.

LC-MS m/z: 701.5 (M+H).

### Example 150

### N-octadecanoyl-γGlu-Ala-Pro-Phe-OH, General Procedure 1

Alternative name: (S)-4-{(S)-2-[(S)-2-((S)-1-Carboxy-2-phenyl-ethylcarbamoyl)-pyrrolidin-1-yl]-1-methyl-2-oxo-ethylcarbamoyl}-2-octadecanoylamino-butyric acid

N-octadecanoyl-γGlu-Ala-Pro-Phe-OH was prepared according to solid phase peptide synthesis—general procedure 1

1H-NMR (300 MHz, AcOD-d4, 80° C., dH): 7.38-7.20 (m, 5H); 5.00-4.52 (m, 4H); 3.93-3.45 (m, 2H); 3.35-3.06 (m, 2H); 2.56-1.93 (m, 10H); 1.77-1.62 (m, 2H); 1.47-1.24 (m, 31H); 0.98-0.86 (m, 3H).

LC-MS purity: 100% (ELSD).

LC-MS Rt (Sunfire 4.6 mm×100 mm, acetonitrile/water 70:30 to 100:0+0.1% FA): 6.68 min.

LC-MS m/z: 729.5 (M+H).

### Example 151

### N-icosanoyl-γGlu-Ala-Pro-Phe-OH, General Procedure 1

Alternative name: (S)-4-{(S)-2-[(S)-2-((S)-1-Carboxy-2-phenyl-ethylcarbamoyl)-pyrrolidin-1-yl]-1-methyl-2-oxo-ethylcarbamoyl}-2-icosanoylamino-butyric acid

N-icosanoyl-γGlu-Ala-Pro-Phe-OH was prepared according to solid phase peptide synthesis—general procedure 1

1H-NMR (300 MHz, AcOD-d4, 80° C., dH): 7.36-7.18 (m, 5H); 4.97-4.52 (m, 4H); 3.93-3.43 (m, 2H); 3.34-3.07 (m, 2H); 2.58-1.93 (m, 10H); 1.76-1.59 (m, 2H); 1.46-1.23 (m, 35H); 0.99-0.85 (m, 3H).

LC-MS purity: 100% (ELSD).

LC-MS Rt (Sunfire 4.6 mm×100 mm, acetonitrile/water 95:5 to 100:0+0.1% FA): 10.33 min.

LC-MS m/z: 757.6 (M+H).

### Example 152

### N-tetradecanoyl-Glu-Ala-Pro-Phe-OH, General Procedure 1

Alternative name: (S)-4-{(S)-2-[(S)-2-((S)-1-Carboxy-2-phenyl-ethylcarbamoyl)-pyrrolidin-1-yl]-1-methyl-2-oxo-ethylcarbamoyl}-4-tetradecanoylamino-butyric acid

N-tetradecanoyl-Glu-Ala-Pro-Phe-OH was prepared according to solid phase peptide synthesis—general procedure 1

1H-NMR (300 MHz, AcOD-d4, 80° C., dH): 7.36-7.14 (m, 5H); 4.99-4.37 (m, 4H); 3.91-3.39 (m, 2H); 3.36-3.02 (m, 2H); 2.48 (t, J=7.4 Hz, 2H); 2.32 (t, J=7.6 Hz, 2H); 2.23-1.88 (m, 6H); 1.74-1.56 (m, 2H); 1.44-1.20 (m, 23H); 0.95-0.85 (m, 3H).

LC-MS purity: 100% (ELSD).

LC-MS Rt (Sunfire 4.6 mm×100 mm, acetonitrile/water 70:30 to 100:0+0.1% FA): 3.88 min.

LC-MS m/z: 673.5 (M+H).

### Example 153

### N-tetradecanoyl-Trp-Pro-Tyr-OH, General Procedure 1

Alternative name: N{Alpha-1}-tetradecanoyl-Trp-Pro-Tyr N-tetradecanoyl-Trp-Pro-Tyr-OH was prepared according to solid phase peptide synthesis—general procedure 1

1H-NMR (300 MHz, AcOD-d4, 80° C., dH): 7.69-7.49 (m, 1H); 7.36 (d, J=7.0 Hz, 1

H); 7.23-6.98 (m, 5H); 6.78 (d, J=7.9 Hz, 2H); 5.25-5.10 (m, 1H); 4.94-4.53 (m, 2H); 3.91-3.72 (m, 1H); 3.45-2.90 (m, 5H); 2.38-2.14 (m, 2H); 2.10-1.76 (m, 4H); 1.74-1.42 (m, 2H); 1.41-1.13 (m, 20H); 0.97-0.83 (m, 3H).

LC-MS purity: 100% (ELSD).

LC-MS Rt (Sunfire 4.6 mm×100 mm, acetonitrile/water 70:30 to 100:0+0.1% FA): 5.08 min.

LC-MS m/z: 674.3 (M+H)⁺.

### Example 154

### N-dodecanoyl-Leu-Thr-Trp-Pro-Tyr-OH, General Procedure 1

Alternative name: N{1}-dodecanoyl-Leu-Thr-Trp-Pro-Tyr-OH N-dodecanoyl-Leu-Thr-Trp-Pro-Tyr-OH was prepared according to solid phase peptide synthesis—general procedure 1

1H-NMR (300 MHz, AcOD-d4, 80° C., dH): 7.70-7.32 (m, 2H), 7.20-7.00 (m, 5H), 6.78 (d, J=6.4 Hz, 2H), 5.21-4.52 (m, 4H), 4.27 (bs, 1H), 3.80-3.42 (m, 1H), 3.40-2.87 (m, 5H), 2.31 (bs, 2H), 1.91-1.78 (m, 1H), 1.78-1.55 (m, 5H), 1.46-1.10 (m, 18H), 1.06-0.79 (m, 9H).

LC-MS purity: 95% (ELSD).

LC-MS Rt (Sunfire 4.6 mm×100 mm, acetonitrile/water 5:95 to 100:0+0.1% FA): 9.08 min.

LC-MS m/z: 861.6 (M+H).

### Example 155

### N-hexadecanoyl-γGlu-DAla-DPro-DPhe-OH, General Procedure 1

Alternative name: N{1}-[(4S)-4-carboxylato-4-(hexadecanoylamino)butanoyl]-D-Ala-D-Pro-D-Phe-OH

N-hexadecanoyl-γGlu-DAla-DPro-DPhe-OH was prepared according to solid phase peptide synthesis—general procedure 1

1H-NMR (300 MHz, AcOD-d4, 80° C.): 7.40-7.14 (m, 5H), 4.97-4.53 (m, 4H), 3.91-3.58 (m, 2H), 3.28-3.07 (m, 2H), 2.51-2.07 (m, 10H), 1.75-1.59 (m, 2H), 1.44-1.24 (m, 27H), 0.98-0.84 (m, 3H).

LC-MS purity: 100%.

LC-MS Rt (Sunfire 4.6 mm×100 mm, acetonitrile/water 70:30 to 100:0+0.1% FA): 6.56 min.

LC-MS m/z: 702.0 (M+H)⁺.

### Example 156

### N-tetradecanoyl-γGlu-D-Ala-D-Ala-D-Pro-D-Phe-OH, General Procedure 1

Alternative name: N{1}-[(4S)-4-carboxylato-4-(tetradecanoylamino)butanoyl]-D-Ala-D-Ala-D-Pro-D-Phe-OH

N-tetradecanoyl-γGlu-D-Ala-D-Ala-D-Pro-D-Phe-OH was prepared according to solid phase peptide synthesis—general procedure 1

1H-NMR (300 MHz, AcOD-d4, 80° C.): 7.37-7.14 (m, 5H), 4.98-4.43 (m, 5H), 3.89-3.51 (m, 2H), 3.36-3.04 (m, 2H), 2.56-2.39 (m, 2H), 2.34 (t, J=8.0 Hz, 2H), 2.29-2.06 (m, 6H), 1.73-1.55 (m, 2H), 1.46-1.21 (m, 26H), 1.01-0.81 (m, 3H).

LC-MS purity: 100%.

LC-MS Rt (Sunfire 4.6 mm×100 mm, acetonitrile/water 70:30 to 100:0+0.1% FA): 3.81 min.

LC-MS m/z: 745.0 (M+H)+.

### Example 157

### N-hexadecanoyl-γGlu-D-Ala-D-Ala-D-Pro-D-Phe-OH, General Procedure 1

Alternative name: N{1}-[(4S)-4-carboxylato-4-(hexadecanoylamino)butanoyl]-D-Ala-D-Ala-D-Pro-D-Phe-OH

N-hexadecanoyl-γGlu-D-Ala-D-Ala-D-Pro-D-Phe-OH was prepared according to solid phase peptide synthesis—general procedure 1

1H-NMR (300 MHz, AcOD-d4, 80° C.): 7.40-7.13 (m, 5H), 4.98-4.51 (m, 5H), 3.87-3.54 (m, 2H), 3.35-3.07 (m, 2H), 2.52-2.39 (m, 2H), 2.34 (t, J=7.82 Hz, 2H), 2.29-2.05 (m, 6H), 1.73-1.59 (m, 2H), 1.47-1.14 (m, 30H), 0.99-0.81 (m, 3H).

LC-MS purity: 100%.

LC-MS Rt (Sunfire 4.6 mm×100 mm, acetonitrile/water 70:30 to 100:0+0.1% FA): 5.92 min.

LC-MS m/z: 773.0 (M+H)⁺.

### Example 158

### N-tetradecanoyl-Thr-Ala-Ala-Pro-Tyr-OH, General Procedure 1

Alternative name: (S)-3-(4-Hydroxy-phenyl)-2-[((S)-1-{(S)-2-[(S)-2-((2S,3R)-3-hydroxy-2-tetradecanoylamino-butyrylamino)-propionylamino]-propionyl}-pyrrolidine-2-carbonyl)-amino]-propionic acid

N-tetradecanoyl-Thr-Ala-Ala-Pro-Tyr-OH was prepared according to solid phase peptide synthesis—general procedure 1

1H-NMR (300 MHz, AcOD, 80° C., dH): 7.20-6.99 (m, 2H); 6.89-6.70 (m, 2H); 4.94-4.47 (m, 5H); 4.43-4.26 (m, 1H); 3.88-3.43 (m, 2H); 3.30-2.94 (m, 2H); 2.39 (t, J=7.4 Hz, 2H); 2.22-1.95 (m, 4H); 1.76-1.58 (m, 2H); 1.46-1.13 (m, 29H); 0.91 (t, J=6.8 Hz, 3H).

LC-MS Purity: 96% (ELSD).

LC-MS Rt (Sunfire 4.6 mm×100 mm, acetonitrile/water 70:30 to 100:0+0.1% FA): 2.72 min.

LC-MS m/z: 732.5 (M+H).

### Example 159

### N-tetradecanoyl-Leu-Ala-Ala-Pro-Tyr-OH, General Procedure 1

Alternative name: (S)-3-(4-Hydroxy-phenyl)-2-[((S)-1-{(S)-2-[(S)-2-((S)-4-methyl-2-tetradecanoylamino-pentanoylamino)-propionylamino]-propionyl}-pyrrolidine-2-carbonyl)-amino]-propionic acid

N-tetradecanoyl-Leu-Ala-Ala-Pro-Tyr-OH was prepared according to solid phase peptide synthesis—general procedure 1

1H-NMR (300 MHz, AcOD, 80° C., dH): 7.19-6.98 (m, 2H); 6.88-6.72 (m, 2H); 4.93-4.44 (m, 5H); 3.90-3.43 (m, 2H); 3.29-2.96 (m, 2H); 2.30 (t, J=7.6 Hz, 2H); 2.20-1.93 (m, 4H); 1.74-1.50 (m, 5H); 1.44-1.22 (m, 26H); 1.01-0.82 (m, 9H).

LC-MS purity: 100% (ELSD).

LC-MS Rt (Sunfire 4.6 mm×100 mm, acetonitrile/water 70:30 to 100:0+0.1% FA):

3.94 min.

LC-MS m/z: 744.5 (M+H).

### Example 160

### N-octadecanoyl-γGlu-Ala-Ala-Pro-Phe-OH, General Procedure 1

Alternative name: (S)-4-((S)-1-{(S)-2-[(S)-2-((S)-1-Carboxy-2-phenyl-ethylcarbamoyl)-pyrrolidin-1-yl]-1-methyl-2-oxo-ethylcarbamoyl}-ethylcarbamoyl)-2-octadecanoylamino-butyric acid

N-octadecanoyl-γGlu-Ala-Ala-Pro-Phe-OH was prepared according to solid phase peptide synthesis—general procedure 1

1H-NMR (300 MHz, AcOD, 80° C., dH): 7.37-7.15 (m, 5H); 5.02-4.47 (m, 5H); 3.94-3.42 (m, 2H); 3.37-3.08 (m, 2H); 2.46 (t, J=7.2 Hz, 2H); 2.39-1.92 (m, 8H); 1.76-1.58 (m, 2

H); 1.48-1.19 (m, 34H); 0.91 (t, J=6.0 Hz, 3H).

LC-MS purity: 100% (ELSD).

LC-MS Rt (SynergiMaxRP 4.6 mm×50 mm, acetonitrile/water 50:50 to 100:0+0.1% FA): 5.03 min.

LC-MS m/z: 800.6 (M+H).

### Example 161

### N-icosanoyl-γGlu-Ala-Ala-Pro-Phe-OH, General Procedure 1

Alternative name: (S)-4-((S)-1-{(S)-2-[(S)-2-((S)-1-Carboxy-2-phenyl-ethylcarbamoyl)-pyrrolidin-1-yl]-1-methyl-2-oxo-ethylcarbamoyl}-ethylcarbamoyl)-2-icosanoylamino-butyric acid

N-icosanoyl-γGlu-Ala-Ala-Pro-Phe-OH was prepared according to solid phase peptide synthesis—general procedure 1

1H-NMR (300 MHz, AcOD, 80° C., dH): 7.37-7.14 (m, 5H); 4.98-4.47 (m, 5H); 3.91-3.42 (m, 2H); 3.36-3.04 (m, 2H); 2.46 (t, J=7.9 Hz, 2H); 2.39-1.92 (m, 8H); 1.76-1.57 (m, 2H); 1.44-1.22 (m, 38H); 0.98-0.83 (m, 3H).

LC-MS purity: 100% (ELSD).

LC-MS Rt (SynergiMaxRP 4.6 mm×50 mm, acetonitrile/water 70:30 to 100:0+0.1% FA): 4.14 min.

LC-MS m/z: 828.7 (M+H).

### Example 162

### N-tetradecanoyl-Glu-Ala-Ala-Pro-Phe-OH, General Procedure 1

Alternative name: (S)-4-((S)-1-{(S)-2-[(S)-2-((S)-1-Carboxy-2-phenyl-ethylcarbamoyl)-pyrrolidin-1-yl]-1-methyl-2-oxo-ethylcarbamoyl}-ethylcarbamoyl)-4-tetradecanoylamino-butyric acid

N-tetradecanoyl-Glu-Ala-Ala-Pro-Phe-OH was prepared according to solid phase peptide synthesis—general procedure 1

1H-NMR (300 MHz, AcOD, 80° C., dH): 7.37-7.14 (m, 5H); 5.00-4.46 (m, 5H); 3.93-3.50 (m, 2H); 3.36-3.04 (m, 2H); 2.48 (t, J=7.3 Hz, 2H); 2.32 (t, J=7.4 Hz, 2H); 2.23-1.91 (m, 6H); 1.64 (t, J=6.3, 2H); 1.47-1.20 (m, 36H); 0.98-0.82 (m, 3H).

LC MS purity: 100% (ELSD)

LC-MS Rt (Sunfire 4.6 mm×100 mm, acetonitrile/water 70:30 to 100:0+0.1% FA): 3.13 min

LC-MS m/z: 744.5 (M+H).

### Example 163

### N-tetradecanoyl-Glu-Ala-Ala-Pro-Phe-OH, General Procedure 1

Alternative name: (S)—N—((S)-1-{(S)-2-[(S)-2-((S)-1-Carboxy-2-phenyl-ethylcarbamoyl)-pyrrolidin-1-yl]-1-methyl-2-oxo-ethylcarbamoyl}-ethyl)-3-tetradecanoylamino-succinamic acid

N-tetradecanoyl-Glu-Ala-Ala-Pro-Phe-OH was prepared according to solid phase peptide synthesis—general procedure 1

1H-NMR (300 MHz, AcOD, 80° C., dH): 7.37-7.15 (m, 5H); 5.03-4.46 (m, 5H); 3.88-3.40 (m, 2H); 3.35-3.03 (m, 2H); 3.01-2.77 (m, 2H); 2.32 (t, J=7.5 Hz, 2H); 2.20-1.90 (m, 4H); 1.73-1.56 (m, 2H); 1.46-1.21 (m, 26H); 0.91 (t, J=6.4 Hz, 3H).

LC-MS purity: 100% (ELSD).

LC-MS Rt (Sunfire 4.6 mm×100 mm, acetonitrile/water 70:30 to 100:0+0.1% FA): 4.26 min.

LC-MS m/z: 730.5 (M+H).

### Example 164

### N-tetradecanoyl-bAsp-Ala-Ala-Pro-Phe-OH, General Procedure 1

Alternative name: (S)—N—((S)-1-{(S)-2-[(S)-2-((S)-1-Carboxy-2-phenyl-ethylcarbamoyl)-pyrrolidin-1-yl]-1-methyl-2-oxo-ethylcarbamoyl}-ethyl)-2-tetradecanoylamino-succinamic acid

N-tetradecanoyl-bAsp-Ala-Ala-Pro-Phe-OH was prepared according to solid phase peptide synthesis—general procedure 1

1H-NMR (300 MHz, AcOD, 80° C., dH): 7.38-7.17 (m, 5H); 4.97-4.46 (m, 5H); 3.89-3.46 (m, 2H); 3.36-3.08 (m, 2H); 3.07-2.81 (m, 2H); 2.34 (t, J=7.6 Hz, 2H); 2.20-1.94 (m, 4H); 1.72-1.57 (m, 2H); 1.42-1.22 (m, 26H); 0.91 (t, J=6.0 Hz, 3H).

LC-MS purity: 100% (ELSD)

LC-MS Rt (Sunfire 4.6 mm×100 mm, acetonitrile/water 70:30 to 100:0+0.1% FA): 4.26 min

LC-MS m/z: 730.5 (M+H).

### Example 165

### N-tetradecanoyl-bAsp-Ala-Pro-Phe-OH, General Procedure 1

Alternative name: (S)—N-{(S)-2-[(S)-2-((S)-1-Carboxy-2-phenylethylcarbamoyl)-pyrrolidin-1-yl]-1-methyl-2-oxo-ethyl}-2-tetradecanoylamino-succinamic acid

N-tetradecanoyl-bAsp-Ala-Pro-Phe-OH was prepared according to solid phase peptide synthesis—general procedure 1

1H-NMR (300 MHz, AcOD-d4, 80° C., dH): 7.36-7.17 (m, 5H); 4.99-4.42 (m, 4H);

3.89-3.40 (m, 2H); 3.38-3.06 (m, 2H); 3.05-2.79 (m, 2H); 2.33 (t, J=7.4 Hz, 2H); 2.20-1.93 (m, 4H); 1.73-1.57 (m, 2H); 1.44-1.23 (m, 23H); 0.96-0.85 (m, 3H).

LC-MS purity: 100% (ELSD).

LC-MS Rt (Sunfire 4.6 mm×100 mm, acetonitrile/water 70:30 to 100:0+0.1% FA): 4.53 min.

LC-MS m/z: 659.5 (M+H).

### Example 166

### N-tetradecanoyl-His-Ala-Ala-Pro-Phe-OH, General Procedure 1

Alternative name: (S)-2-{[(S)-1-((S)-2-{(S)-2-[(S)-3-(3H-Imidazol-4-yl)-2-tetradecanoylaminopropionylamino]-propionylamino}-propionyl)-pyrrolidine-2-carbonyl]-amino}-3-phenylpropionic acid

N-tetradecanoyl-His-Ala-Ala-Pro-Phe-OH was prepared according to solid phase peptide synthesis—general procedure 1

1H-NMR (300 MHz, AcOD, 80° C., dH): 8.74 (br. s, 1H); 7.46-7.10 (m, 6H); 5.09-4.42 (m, 5H); 3.95-3.45 (m, 2H); 3.45-3.05 (m, 4H); 2.30 (t, J=7.4 Hz, 2H); 2.22-1.91 (m, 4H); 1.68-1.52 (m, 2H); 1.45-1.21 (m, 26H); 0.90 (t, J=6.0 Hz, 3H).

LC-MS purity: 100% (ELSD).

LC-MS Rt (Sunfire 4.6 mm×100 mm, acetonitrile/water 35:65 to 100:0+0.1% FA): 4.49 min.

LC-MS m/z: 752.6 (M+H). (1A)

### Example 167

### PEG12-Ala-Ala-Pro-Phe-OH, General Procedure 1

Alternative name: N{1}-3-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]ethoxy]ethoxy]propanoyl-Ala-Ala-Pro-Phe-OH

PEG12-Ala-Ala-Pro-Phe-OH was prepared according to solid phase peptide synthesis—general procedure 1

1H-NMR (300 MHz, AcOD, 80° C., dH): 7.36-7.16 (m, 5H); 5.01-4.49 (m, 4H); 3.91-3.54 (m, 48H); 3.40 (s, 3H); 3.33-3.06 (m, 2H); 2.59 (t, J=6.0 Hz, 2H); 2.21-1.94 (m, 4H); 1.42-1.27 (m, 6H).

LC-MS purity: 100% (ELSD).

LC-MS Rt (Sunfire 4.6 mm×100 mm, acetonitrile/water 35:65 to 100:0+0.1% FA): 2.29 min.

LC-MS m/z: 975.8 (M+H).

### Example 168

### N-tetradecanoyl-γGlu-Ala-Pro-D-Phe-OH, General Procedure 1

Alternative name: N{1}-[(4S)-4-carboxy-4-(tetradecanoylamino)butanoyl]-Ala-Pro-D-Phe-OH

N-tetradecanoyl-γGlu-Ala-Pro-D-Phe-OH was prepared according to solid phase peptide synthesis—general procedure 1

1H-NMR (300 MHz, AcOD-d4, 80° C.): 7.36-7.13 (m, 5H), 4.96-4.52 (m, 4H), 3.85-2.99 (m, 4H), 2.50-2.05 (m, 10H), 1.72-1.60 (m, 2H), 1.42-1.24 (m, 23H), 0.97-0.83 (m, 3H).

LC-MS purity: 100%.

LC-MS Rt (Sunfire 4.6 mm×100 mm, acetonitrile/water 70:30 to 100:0+0.1% FA):

3.65 min.

LC-MS m/z: 674.0 (M+H)⁺.

### Example 169

### N-tetradecanoyl-γGlu-γGlu-Ala-Pro-Phe-OH, General Procedure 1

Alternative name: N{1}-[(4S)-4-carboxy-4-[[(4S)-4-carboxy-4-(tetradecanoylamino)butanoyl]amino]butanoyl]-Ala-Pro-Phe-OH

N-tetradecanoyl-γGlu-γGlu-Ala-Pro-Phe-OH was prepared according to solid phase peptide synthesis—general procedure 1

1H-NMR (300 MHz, AcOD-d4, 80° C.): 7.38-7.10 (m, 5H), 5.00-4.51 (m, 5H), 3.88-3.10 (m, 4H), 2.58-2.06 (m, 14H), 1.74-1.60 (m, 2H), 1.32 (s, 23H), 0.97-0.82 (m, 3H).

LC-MS purity: 98%.

LC-MS Rt (Sunfire 4.6 mm×100 mm, acetonitrile/water 70:30 to 100:0+0.1% FA): 3.51 min.

LC-MS m/z: 803.0 (M+H)⁺.

### Example 170

### N-tetradecanoyl-γGlu-γGlu-Pro-Phe-OH, General Procedure 1

Alternative name: N{1}-[(4S)-4-carboxy-4-[[(4S)-4-carboxy-4-(tetradecanoylamino)butanoyl]amino]butanoyl]-Pro-Phe-OH

N-tetradecanoyl-γGlu-γGlu-Pro-Phe-OH was prepared according to solid phase peptide synthesis—general procedure 1

1H-NMR (300 MHz, AcOD-d4, 80° C.): 7.37-7.17 (m, 5H), 5.04-4.47 (m, 4H), 3.63-3.05 (m, 4H), 2.64-2.06 (m, 14H), 1.73-1.60 (m, 2H), 1.43-1.26 (m, 20H), 0.90 (t, J=6.31 Hz, 3H).

LC-MS purity: 100%.

LC-MS Rt (Sunfire 4.6 mm×100 mm, acetonitrile/water 70:30 to 100:0+0.1% FA): 4.45 min.

LC-MS m/z: 732.0 (M+H)⁺.

### Example 171

### N-tetradecanoyl-γGlu-γGlu-Phe-Phe-OH, General Procedure 1

Alternative name: N{1}-[(4S)-4-carboxy-4-[[(4S)-4-carboxy-4-(tetradecanoylamino)butanoyl]amino]butanoyl]-Phe-Phe-OH

N-tetradecanoyl-γGlu-γGlu-Phe-Phe-OH was prepared according to solid phase peptide synthesis—general procedure 1

1H-NMR (300 MHz, AcOD-d4, 80° C.): 7.31-7.12 (m, 10H), 4.91-4.49 (m, 4H), 3.29-2.88 (m, 4H), 2.56-2.07 (m, 10H), 1.72-1.60 (m, 2H), 1.32 (s, 20H), 0.90 (t, J=6.69 Hz, 3H).

LC-MS purity: 99%.

LC-MS Rt (Sunfire 4.6 mm×100 mm, acetonitrile/water 70:30 to 100:0+0.1% FA): 4.73 min.

LC-MS m/z: 782.0 (M+H)⁺.

### Example 172

### N-dodecanoyl-Thr-Ala-Ala-Pro-Phe-OH, General Procedure 1

Alternative name: N{1}-dodecanoyl-Thr-Ala-Ala-Pro-Phe-OH

N-dodecanoyl-Thr-Ala-Ala-Pro-Phe-OH was prepared according to solid phase peptide synthesis—general procedure 1

1H-NMR (300 MHz, AcOD-d4, 80° C., dH): 7.35-7.16 (m, 5H); 4.96-4.70 (m, 2H); 4.69-4.47 (m, 3H); 4.42-4.26 (m, 1H); 3.86-3.69 (m, 1H); 3.70-3.53 (m, 1H); 3.33-3.05 (m, 2H); 2.29-2.46 (m, 2H); 2.20-1.89 (m, 4H); 1.74-1.56 (m, 2H); 1.49-1.05 (m, 25H); 0.96-0.83 (m, 3H).

LC-MS purity: 96% (ELSD).

LC-MS Rt (Sunfire 4.6 mm×100 mm, acetonitrile/water 70:30 to 100:0+0.1% TFA): 2.84 min.

LC-MS m/z: 687.0 (M+H)⁺.

### Example 173

### N-tetradecanoyl-Thr-Ala-Ala-Pro-Phe-OH, General Procedure 1

Alternative name: N{1}-tetradecanoyl-Thr-Ala-Ala-Pro-Phe-OH

N-tetradecanoyl-Thr-Ala-Ala-Pro-Phe-OH was prepared according to solid phase peptide synthesis—general procedure 1

1H-NMR (300 MHz, AcOD-d4, 80° C., dH): 7.35-7.17 (m, 5H); 4.96-4.72 (m, 2H); 4.69-4.49 (m, 3H); 4.41-4.27 (m, 1H); 3.86-3.71 (m, 1H); 3.70-3.55 (m, 1H); 3.32-3.05 (m, 2H); 2.45-2.31 (m, 2H); 2.20-1.90 (m, 4H); 1.75-1.57 (m, 2H); 1.47-1.14 (m, 29H); 0.95-0.84 (m, 3H).

LC-MS purity: 95% (ELSD).

LC-MS Rt (Sunfire 4.6 mm×100 mm, acetonitrile/water 70:30 to 100:0+0.1% TFA): 4.44 min.

LC-MS m/z: 715.4 (M+H)⁺.

### Example 174

### N-tetradecanoyl-Glu-Ala-Ala-Pro-D-Phe-OH, General Procedure 1

Alternative name: N{1}-tetradecanoyl-Glu-Ala-Ala-Pro-D-Phe-OH

N-tetradecanoyl-Glu-Ala-Ala-Pro-D-Phe-OH was prepared according to solid phase peptide synthesis—general procedure 1

1H-NMR (300 MHz, AcOD-d4, 80° C., dH): 7.35-7.15 (m, 5H); 4.97-4.85 (m, 1H); 4.85-4.72 (m, 1H); 4.72-4.45 (m, 3H); 3.83-3.69 (m, 1H); 3.68-3.52 (m, 1H); 3.35-3.19 (m, 1H); 3.16-2.97 (m, 1H); 2.54-2.40 (m, 2H); 2.37-2.23 (m, 2H); 2.22-1.79 (m, 6H); 1.72-1.53 (m, 2H); 1.43-1.14 (m, 26H); 0.96-0.83 (m, 3H).

LC-MS purity: 100% (ELSD).

LC-MS Rt (Sunfire 4.6 mm×100 mm, acetonitrile/water 70:30 to 100:0+0.1% FA): 3.16 min.

LC-MS m/z: 743.5 (M+H)⁺.

### Example 175

### N-hexadecanoyl-Thr-Ala-Ala-Pro-Phe-OH, General Procedure 1

Alternative name: N{1}-hexadecanoyl-Thr-Ala-Ala-Pro-Phe-OH

N-hexadecanoyl-Thr-Ala-Ala-Pro-Phe-OH was prepared according to solid phase peptide synthesis—general procedure 1

1H-NMR (300 MHz, AcOD-d4, 80° C., dH): 7.36-7.14 (m, 5H); 4.96-4.85 (m, 1H); 4.85-4.70 (m, 1H); 4.68-4.48 (m, 3H); 4.40-4.28 (m, 1H); 3.87-3.70 (m, 1H); 3.70-3.54 (m, 1H); 3.33-3.05 (m, 2H); 2.44-2.30 (m, 2H); 2.20-1.86 (m, 4H); 1.76-1.57 (m, 2H); 1.49-1.02 (m, 33H); 0.97-0.82 (m, 3H).

LC-MS purity: 96% (ELSD).

LC-MS Rt (Sunfire 4.6 mm×100 mm, acetonitrile/water 70:30 to 100:0+0.1% TFA): 5.60 min.

LC-MS m/z: 743.4 (M+H)⁺.

### Example 176

### N-tetradecanoyl-γGlu-Ala-Pro-Trp-OH, General Procedure 1

Alternative name: N{1}-[(4S)-4-carboxy-4-(tetradecanoylamino)butanoyl]-Ala-Pro-Trp-OH

N-tetradecanoyl-γGlu-Ala-Pro-Trp-OH was prepared according to solid phase peptide synthesis—general procedure 1

1H-NMR (300 MHz, AcOD-d4, 80° C.): 7.80-6.81 (m, 6H), 5.10-4.52 (m, 4H), 3.89-3.31 (m, 4H), 2.53-2.06 (m, 10H), 1.75-1.55 (m, 2H), 1.34-1.14 (m, 23H), 1.01-0.85 (m, 3H).

LC-MS purity: 100%.

LC-MS Rt (Sunfire 4.6 mm×100 mm, acetonitrile/water 70:30 to 100:0+0.1% FA): 4.02 min.

LC-MS m/z: 712.0 (M+H)⁺.

### Example 177

### N-tetradecanoyl-γGlu-Ala-Pro-D-Trp-OH, General Procedure 1

Alternative name: N{1}-[(4S)-4-carboxy-4-(tetradecanoylamino)butanoyl]-Ala-Pro-D-Trp-OH

N-tetradecanoyl-γGlu-Ala-Pro-D-Trp-OH was prepared according to solid phase peptide synthesis—general procedure 1

1H-NMR1H NMR spectrum (300 MHz, AcOD-d4, 80 C): 7.74-6.93 (m, 6H), 5.01-4.52 (m, 4H), 3.94-3.18 (m, 4H), 2.53-1.87 (m, 10H), 1.75-1.59 (m, 2H), 1.43-1.20 (m, 23H), 0.97-0.89 (m, 3H).

LC-MS purity: 97%.

LC-MS Rt (Sunfire 4.6 mm×100 mm, acetonitrile/water 70:30 to 100:0+0.1% FA): 4.04 min.

LC-MS m/z: 713.0 (M+H)+.

### Example 178

### N-tetradecanoyl-His-Ala-Arg-Pro-Phe-OH, General Procedure 1

Alternative name: N{Alpha-1}-tetradecanoyl-His-Ala-Arg-Pro-Phe-OH

N-tetradecanoyl-His-Ala-Arg-Pro-Phe-OH was prepared according to solid phase peptide synthesis—general procedure 1

1H-NMR (300 MHz, AcOD-d4, 80° C., H): 8.83-8.64 (m, 1H); 7.42-7.16 (m, 6H); 5.07-4.70 (m, 3H); 4.70-4.42 (m, 2H); 3.89-3.04 (m, 8H); 2.39-1.66 (m, 12H); 1.66-1.03 (m, 23H); 0.96-0.83 (m, 3H).

LC-MS purity: 100% (ELSD).

LC-MS Rt (Sunfire 4.6 mm×100 mm, acetonitrile/water 70:30 to 100:0+0.1% FA): 0.87 min.

LC-MS m/z: 836.5 (M+H)+.

### Example 179

### N-tetradecanoyl-γGlu-Ala-Arg-Pro-Phe-OH, General Procedure 1

Alternative name: N{1}-[(4S)-4-carboxy-4-(tetradecanoylamino)butanoyl]-Ala-Arg-Pro-Phe-OH

N-tetradecanoyl-γGlu-Ala-Arg-Pro-Phe-OH was prepared according to solid phase peptide synthesis—general procedure 1

1H-NMR (300 MHz, AcOD-d4, 80° C., dH): 7.36-7.19 (m, 5H); 4.91-4.75 (m, 2H); 4.68-4.46 (m, 3H); 3.88-3.74 (m, 1H); 3.73-3.60 (m, 1H); 3.34-3.08 (m, 4H); 2.53-2.40 (m, 2H); 2.40-2.29 (m, 2H); 2.29-1.87 (m, 6H); 1.87-1.57 (m, 6H); 1.45-1.22 (m, 23H); 0.97-0.84 (m, 3H).

LC-MS purity: 100% (ELSD).

LC-MS Rt (Sunfire 4.6 mm×100 mm, acetonitrile/water 70:30 to 100:0+0.1% FA): 1.24 min.

LC-MS m/z: 828.5 (M+H)+.

### Example 180

### N-dodecanoyl-Ala-Ala-Pro-His-OH, General Procedure 1

Alternative name: N{1}-dodecanoyl-Ala-Ala-Pro-His-OH

N-dodecanoyl-Ala-Ala-Pro-His-OH was prepared according to solid phase peptide synthesis—general procedure 1

1H-NMR (300 MHz, AcOD-d4, 80° C.): 9.14-8.29 (m, 1H), 7.57-7.31 (m, 1H), 5.14-4.41 (m, 4H), 3.94-3.17 (m, 4H), 2.43-2.11 (m, 6H), 1.70-1.55 (m, 2H), 1.48-1.14 (m, 22H), 1.01-0.76 (m, 3H).

LC-MS purity: 100%.

LC-MS Rt (Sunfire 4.6 mm×100 mm, acetonitrile/water 35:65 to 100:0+0.1% FA): 4.12 min.

LC-MS m/z: 578.0 (M+H)⁺.

### Example 181

### N-tetradecanoyl-γGlu-Ala-Pro-Phe-OH, General Procedure 1

Alternative name: (S)-4-{(S)-2-[(S)-2-((S)-1-Carboxy-2-phenyl-ethylcarbamoyl)-pyrrolidin-1-yl]-1-methyl-2-oxo-ethylcarbamoyl}-2-tetradecanoylamino-butyric acid

N-tetradecanoyl-γGlu-Ala-Pro-Phe-OH was prepared according to solid phase peptide synthesis—general procedure 1

1H-NMR1H NMR spectrum (300 MHz, AcOD-d4 dH): 7.34-7.17 (m, 5H); 5.00-4.397 (m, 4H); 3.91-3.38 (m, 2H); 3.39-3.02 (m, 2H); 2.56-1.91 (m, 10H); 1.76-1.59 (m, 2H); 1.48-1.20 (m, 23H); 0.95-0.84 (m, 3H).

LC-MS purity: 99%.

LC-MS Rt (Sunfire 4.6 mm×100 mm, acetonitrile/water 70:30 to 100:0+0.1% FA): 4.76 min.

LC-MS m/z: 673.7 (M+H)⁺.

### Example 182

### N-hexadecanoyl-γGlu-D-Ala-D-Pro-D-Phe-OH, General Procedure 1

Alternative name: N{1}-[(4S)-4-carboxy-4-(hexadecanoylamino)butanoyl]-D-Ala-D-Pro-D-Phe-OH

### N-hexadecanoyl-γGlu-D-Ala-D-Pro-D-Phe-OH was prepared according to solid phase peptide synthesis—general procedure 1

1H-NMR (300 MHz, AcOD-d4, 80° C.): 7.36-7.15 (m, 5H), 4.98-4.37 (m, 4H), 3.89-3.06 (m, 4H), 2.53-2.05 (m, 10H), 1.73-1.57 (m, 2H), 1.40-1.21 (m, 27H), 0.94-0.84 (m, 3H).

LC-MS purity: 96%.

LC-MS Rt (Sunfire 4.6 mm×100 mm, acetonitrile/water 70:30 to 100:0+0.1% FA):

5.28 min.

LC-MS m/z: 702 (M+H)⁺.

### Example 183

### N-tetradecanoyl-γGlu-D-Ala-D-Pro-D-Phe-OH, General Procedure 1

Alternative name: N{1}-[(4S)-4-carboxy-4-(tetradecanoylamino)butanoyl]-D-Ala-D-Pro-D-Phe-OH

N-tetradecanoyl-γGlu-D-Ala-D-Pro-D-Phe-OH was prepared according to solid phase peptide synthesis—general procedure 1

1H-NMR (300 MHz, AcOD-d4, 80° C.): 7.34-7.16 (m, 5H), 4.95-4.56 (m, 4H), 3.88-3.09 (m, 4H), 2.52-2.06 (m, 10H), 1.75-1.59 (m, 2H), 1.45-1.24 (m, 23H), 0.97-0.84 (m, 3H).

LC-MS purity: 100%.

LC-MS Rt (Sunfire 4.6 mm×100 mm, acetonitrile/water 70:30 to 100:0+0.1% FA): 4.71 min.

LC-MS m/z: 674 (M+H)+.

### Example 184

### N-tetradecanoyl-D-Ala-D-Ala-D-Pro-D-Phe-OH, General Procedure 1

Alternative name: N{1}-tetradecanoyl-D-Ala-D-Ala-D-Pro-D-Phe-OH

N-tetradecanoyl-D-Ala-D-Ala-D-Pro-D-Phe-OH was prepared according to solid phase peptide synthesis—general procedure 1

1H-NMR (300 MHz, AcOD-d4, 80° C.): 7.34-7.15 (m, 5H), 4.95-4.53 (m, 4H), 3.86-3.06 (m, 4H), 2.35-2.06 (m, 6H), 1.70-1.56 (m, 2H), 1.41-1.21 (m, 26H), 0.96-0.83 (m, 3H).

LC-MS purity: 100%.

LC-MS Rt (Sunfire 4.6 mm×100 mm, acetonitrile/water 70:30 to 100:0+0.1% FA): 4.79 min.

LC-MS m/z: 616 (M+H)+.

### Example 185

### N-tetradecanoyl-Ala-Ala-Pro-D-Phe-OH, General Procedure 1

Alternative name: (R)-3-Phenyl-2-({(S)-1-[(S)-2-((S)-2-tetradecanoylamino-propionylamino)-propionyl]-pyrrolidine-2-carbonyl}-amino)-propionic acid

N-tetradecanoyl-Ala-Ala-Pro-D-Phe-OH was prepared according to solid phase peptide synthesis—general procedure 1

1H-NMR (300 MHz, AcOD-d4, 80° C., dH): 7.35-7.14 (m, 5H); 4.99-4.85 (m, 1H); 4.83-4.70 (m, 1H); 4.67-4.53 (m, 2H); 3.86-3.71 (m, 1H); 3.68-3.53 (m, 1H); 3.34-3.21 (m, 1H); 3.14-2.99 (m, 1H); 2.35-2.22 (m, 2H); 2.12-1.79 (m, 4H); 1.71-1.54 (m, 2H); 1.44-1.17 (m, 26H); 0.95-0.85 (m, 3H).

LC-MS purity: 100% (ELSD).

LC-MS Rt (Sunfire 4.6 mm×100 mm, acetonitrile/water 70:30 to 100:0+0.1% FA): 5.34 min.

LC-MS m/z: 615.0 (M+H)⁺.

### Example 186

### N-hexadecanoyl-Glu-Ala-Ala-Pro-D-Phe-OH, General Procedure 1

Alternative name: N{1}-hexadecanoyl-Glu-Ala-Ala-Pro-D-Phe-OH

N-hexadecanoyl-Glu-Ala-Ala-Pro-D-Phe-OH was prepared according to solid phase peptide synthesis—general procedure 1

1H-NMR (300 MHz, AcOD-d4, 80° C., dH): 7.33-7.19 (m, 5H); 4.96-4.87 (m, 1H); 4.84-4.74 (m, 1H); 4.72-4.48 (m, 3H); 3.85-3.70 (m, 1H); 3.68-3.55 (m, 1H); 3.33-3.22 (m, 1H); 3.13-3.00 (m, 1H); 2.48 (t, J=7.4 Hz, 2H); 2.31 (t, J=7.5 Hz, 2H); 2.24-1.81 (m, 6H); 1.71-1.58 (m, 2H); 1.42-1.24 (m, 30H); 0.94-0.86 (m, 3H).

LC-MS purity: 100% (ELSD).

LC-MS Rt (Sunfire 4.6 mm×100 mm, acetonitrile/water 70:30 to 100:0+0.1% FA): 5.62 min.

LC-MS m/z: 771.0 (M+H)⁺.

### Example 187

### N-tetradecanoyl-Glu-Ala-Ala-Pro-Trp-OH, General Procedure 1

Alternative name: N{1}-tetradecanoyl-Glu-Ala-Ala-Pro-Trp-OH N-tetradecanoyl-Glu-Ala-Ala-Pro-Trp-OH was prepared according to solid phase peptide synthesis—general procedure 1

1H-NMR1H NMR spectrum (300 MHz, AcOD-d4, 80 C, dH): 7.61 (d, J=7.5 Hz, 1H); 7.36 (d, J=8.1 Hz, 1H); 7.21-7.00 (m, 3H); 5.01-4.90 (m, 1H); 4.81-4.46 (m, 4H); 3.83-3.66 (m, 1H); 3.59-3.29 (m, 3H); 2.48 (t, J=7.4 Hz, 2H); 2.32 (t, J=7.4 Hz, 2H); 2.24-1.81 (m, 6H); 1.72-1.57 (m, 2H); 1.41-1.19 (m, 26H); 0.95-0.86 (m, 3H).

LC-MS purity: 100% (ELSD).

LC-MS Rt (Sunfire 4.6 mm×100 mm, acetonitrile/water 70:30 to 100:0+0.1% FA): 3.69 min.

LC-MS m/z: 782.0 (M+H)⁺.

### Example 188

### N-hexadecanoyl-γGlu-Ala-Ala-Pro-Phe-OH, General Procedure 1

Alternative name: (S)-4-((S)-1-{(S)-2-[(S)-2-((S)-1-Carboxy-2-phenyl-ethylcarbamoyl)-pyrrolidin-1-yl]-1-methyl-2-oxo-ethylcarbamoyl}-ethylcarbamoyl)-2-hexadecanoylamino-butyric acid

N-hexadecanoyl-γGlu-Ala-Ala-Pro-Phe-OH was prepared according to solid phase peptide synthesis—general procedure 1

1H-NMR1H NMR spectrum (300 MHz, AcOD-d4, 80 C, dH): 7.39-7.12 (m, 5H); 4.99-4.36 (m, 5H); 3.92-3.39 (m, 2H); 3.40-2.96 (m, 2H); 2.60-1.86 (m, 10H); 1.77-1.53 (m, 2H); 1.46-1.09 (m, 30H); 0.92 (m, 3H).

LC-MS purity: 98%.

LC-MS Rt (Sunfire 4.6 mm×100 mm, acetonitrile/water 70:30 to 100:0+0.1% FA): 5.73 min.

LC-MS m/z: 772.0 (M+H)+.

### Example 189

### N-icosanoyl-γGlu-Ala-Pro-Phe-OH, General Procedure 1

Alternative name: (S)-4-{(S)-2-[(S)-2-((S)-1-Carboxy-2-phenyl-ethylcarbamoyl)-pyrrolidin-1-yl]-1-methyl-2-oxo-ethylcarbamoyl}-2-icosanoylamino-butyric acid

N-icosanoyl-γGlu-Ala-Pro-Phe-OH was prepared according to solid phase peptide synthesis—general procedure 1

1H-NMR1H NMR spectrum (300 MHz, AcOD-d4 dH): 7.37-7.17 (m, 5H); 4.99-4.51 (m, 4H); 3.95-3.02 (m, 4H); 2.60-1.88 (m, 10H); 1.78-1.58 (m, 2H); 1.43-1.25 (m, 35H); 0.92 (t, J=6.4 Hz, 3H).

LC-MS purity: 96%.

LC-MS Rt (Synergi max-RP 4.6 mm×50 mm, acetonitrile/water 70:30 to 100:0+0.1% FA): 5.21 min.

LC-MS m/z: 756.6 (M+H)+.

### Example 190

### N-dodecanoyl-γGlu-His-Ala-Ala-Pro-Tyr-OH, General Procedure 1

Alternative name: N{Alpha-1}-[(4S)-4-carboxy-4-(dodecanoylamino)butanoyl]-His-Ala-Ala-Pro-Tyr-OH

N-dodecanoyl-γGlu-His-Ala-Ala-Pro-Tyr-OH was prepared according to solid phase peptide synthesis—general procedure 1

1H-NMR (300 MHz, AcOD-d4, 80° C., dH): 8.76-8.67 (m, 1H); 7.43-7.34 (m, 1H); 7.08 (d, J=7.7 Hz, 2H); 6.79 (d, J=8.3 Hz, 2H); 5.01-4.72 (m, 3H); 4.66-4.40 (m, 3H); 3.89-3.72 (m, 1H); 3.70-3.57 (m, 1H); 3.45-3.31 (m, 1H); 3.30-2.99 (m, 3H); 2.53-2.40 (m, 2H); 2.39-2.27 (m, 2H); 2.25-1.79 (m, 6H); 1.74-1.56 (m, 2H); 1.53-1.11 (m, 22H); 0.94-0.86 (m, 3H).

LC-MS purity: 100% (ELSD).

LC-MS Rt (Sunfire 4.6 mm×100 mm, acetonitrile/water 5:95 to 100:0+0.1% FA): 6.41 min.

LC-MS m/z: 869.0 (M+H)+.

### Example 191

### N-tetradecanoy-γGlu-His-Ala-Ala-Pro-Tyr-OH, General Procedure 1

Alternative name: N{Alpha-1}-[(4S)-4-carboxy-4-(tetradecanoylamino)butanoyl]-His-Ala-Ala-Pro-Tyr-OH

N-tetradecanoy-γGlu-His-Ala-Ala-Pro-Tyr-OH was prepared according to solid phase peptide synthesis—general procedure 1

1H-NMR (300 MHz, AcOD-d4, 80° C., dH): 8.76-8.66 (m, 1H); 7.44-7.33 (m, 1H); 7.08 (d, J=7.4 Hz, 2H); 6.79 (d, J=7.5 Hz, 2H); 5.02-4.70 (m, 3H); 4.67-4.41 (m, 3H); 3.87-3.71 (m, 1H); 3.70-3.59 (m, 1H); 3.47-2.97 (m, 4H); 2.53-2.39 (m, 2H); 2.39-2.28 (m, 2H); 2.23-1.76 (m, 6H); 1.74-1.56 (m, 2H); 1.51-1.11 (m, 26H); 0.94-0.86 (m, 3H).

LC-MS purity: 100% (ELSD).

LC-MS Rt (Sunfire 4.6 mm×100 mm, acetonitrile/water 5:95 to 100:0+0.1% FA): 7.02 min.

LC-MS m/z: 897.0 (M+H)+.

### Example 192

### N-tetradecanoyl-His-Ala-Trp-Pro-Phe-OH, General Procedure 1

Alternative name: N{Alpha-1}-tetradecanoyl-His-Ala-Trp-Pro-Phe-OH

N-tetradecanoyl-His-Ala-Trp-Pro-Phe-OH was prepared according to solid phase peptide synthesis—general procedure 1

1H-NMR (300 MHz, AcOD-d4, 80° C., dH): 8.79-8.67 (m, 1H); 7.69-7.58 (m, 1H); 7.40-7.01 (m, 10H); 5.21-5.04 (m, 1H); 5.02-4.70 (m, 2H); 4.69-4.42 (m, 2H); 3.83-2.99 (m, 8H); 2.38-2.18 (m, 2H); 2.17-1.73 (m, 4H); 1.70-1.00 (m, 25H); 0.95-0.83 (m, 3H).

LC-MS Purity: 100% (ELSD).

LC-MS Rt (Sunfire 4.6 mm×100 mm, acetonitrile/water 5:95 to 100:0+0.1% FA): 7.56 min.

LC-MS m/z: 867.0 (M+H)+

### Example 193

### N-tetradecanoyl-Lys-Ala-Arg-Pro-Phe-OH, General Procedure 1

Alternative name: (S)-2-[((S)-1-{(S)-2-[(S)-2-((S)-6-Amino-2-tetradecanoylamino-hexanoylamino)-propionylamino]-5-guanidino-pentanoyl}-pyrrolidine-2-carbonyl)-amino]-3-phenyl-propionic acid

N-tetradecanoyl-Lys-Ala-Arg-Pro-Phe-OH was prepared according to solid phase peptide synthesis—general procedure 1

1H-NMR (300 MHz, AcOD-d4, 80° C., dH): 7.36-7.21 (m, 5H); 4.96-4.43 (m, 5H); 3.89-3.60 (m, 2H); 2.32 (t, J=7.4 Hz, 2H); 2.25-1.45 (m, 16H); 1.44-1.25 (m, 23H); 0.97-0.85 (m, 3H).

LC-MS purity: 95% (ELSD).

LC-MS Rt (Sunfire 4.6 mm×100 mm, acetonitrile/water 5:95 to 100:0+0.1% TFA): 6.39 min.

LC-MS m/z: 828.9 (M+H).

### Example 194

### N-tetradecanoyl-γGlu-His-Ala-Arg-Pro-Phe-OH, General Procedure 1

Alternative name: N{Alpha-1}-[(4S)-4-carboxy-4-(tetradecanoylamino)butanoyl]-His-Ala-Arg-Pro-Phe-OH

N-tetradecanoyl-γGlu-His-Ala-Arg-Pro-Phe-OH was prepared according to solid phase peptide synthesis—general procedure 1

1H-NMR1H NMR spectrum (300 MHz, AcOD-d4, 80 C): 8.80-8.61 (m, 1H), 7.47-7.17 (m, 6H), 4.99-4.43 (m, 6H), 3.78-3.50 (m, 6H), 3.31-3.19 (m, 2H), 2.53-2.13 (m, 10H), 1.71-1.58 (m, 2H), 1.39-1.24 (m, 27H), 0.98-0.82 (m, 3H).

LC-MS purity: 100%.

LC-MS Rt (Sunfire 4.6 mm×100 mm, acetonitrile/water 20:80 to 100:0+0.1% FA): 5.05 min.

LC-MS m/z: 484.0 (M+H)+/2.

### Example 195

### N-tetradecanoyl-D-His-D-Ala-D-Arg-D-Pro-D-Phe-OH, General procedure 1

Alternative name: N{Alpha-1}-tetradecanoyl-D-His-D-Ala-D-Arg-D-Pro-D-Phe-OH

N-tetradecanoyl-D-His-D-Ala-D-Arg-D-Pro-D-Phe-OH was prepared according to solid phase peptide synthesis—general procedure 1

1H-NMR1H NMR spectrum (300 MHz, AcOD-d4, 80 C): 8.80 (bs, 1H), 7.35-7.22 (m, 6H), 5.04-4.47 (m, 5H), 3.91-3.10 (m, 8H), 2.45-2.08 (m, 6H), 1.98-1.81 (m, 2H), 1.75-1.20 (m, 27H), 0.93-0.83 (m, 3H).

LC-MS purity: 100%.

LC-MS Rt (Sunfire 4.6 mm×100 mm, acetonitrile/water 20:80 to 100:0+0.1% FA): 4.86 min.

LC-MS m/z: 838.0 (M+H)+.

### Example 196

### N-tetradecanoyl-eLys-His-Ala-Arg-Pro-Phe-OH, General Procedure 1

Alternative name: N{Epsilon}-tetradecanoylLys-His-Ala-Arg-Pro-Phe-OH

N-tetradecanoyl-eLys-His-Ala-Arg-Pro-Phe-OH was prepared according to solid phase peptide synthesis—general procedure 1

1H NMR spectrum (300 MHz, AcOD-d4, 80 C): 8.78-8.62 (m, 1H), 7.45-7.21 (m, 6H), 4.89-4.19 (m, 6H), 3.49-3.17 (m, 8H), 2.32-2.07 (m, 12H), 1.99-1.89 (m, 2H), 1.85-1.54 (m, 6H), 1.52-1.26 (m, 27H), 0.97-0.84 (m, 3H).

LC-MS purity: 97%.

LC-MS Rt (Sunfire 4.6 mm×100 mm, acetonitrile/water 20:80 to 100:0+0.1% FA): 4.86 min.

LC-MS m/z: 484.0 (M+H)+/2.

### Example 197

### N-tetradecanoyl-Arg-His-Ala-Arg-Pro-Phe-OH, General Procedure 1

Alternative name: N{Alpha-1}-tetradecanoyl-Arg-His-Ala-Arg-Pro-Phe-OH

N-tetradecanoyl-Arg-His-Ala-Arg-Pro-Phe-OH was prepared according to solid phase peptide synthesis—general procedure 1

1H-NMR (300 MHz, AcOD-d4, 80° C.): 8.80-8.70 (m, 1H), 7.43-7.21 (m, 6H), 4.94-4.44 (m, 6H), 3.83-3.19 (m, 10H), 2.34-2.18 (m, 6H), 1.99-1.92 (m, 2H), 1.80-1.23 (m, 31H), 0.94-0.84 (m, 3H).

LC-MS purity: 100%.

LC-MS Rt (Sunfire 4.6 mm×100 mm, acetonitrile/water 20:80 to 100:0+0.1% FA): 4.62 min.

LC-MS m/z: 498.0 (M+H)+/2.

### Example 198

### Inhibition of Enzymatic Degradation of a Model GLP-1

The use of Förster resonance energy transfer (FRET), also known as fluorescence resonance energy transfer, substrates to monitor activity of proteolytic enzymes is known in the field (for example Anjuere, F. et al. (1993). Biochem J 291 (Pt 3), 869-73).

Model GLP-1 analogue was designed as FRET substrate by incorporation of 7-Methoxycoumarin-4-acetic acid (MCA) group as the donor chromophore and dinitrophenol group (DNP) as the acceptor chromophore.

An assay following the increase in fluorescence as a function of time was established in 96 well format using Varioskan Flash Multimode Meter (Thermo Scientific). Each well contained 70 μl of Dulbecco's phosphate buffer saline (Invitrogen catalogue #14190-094), 10 μl of 100 μM GLP-1 FRET substrate, 10 μl of N-terminally acylated peptide or oligopeptide of the invention in varying concentration and 10 μl of a stock solution of an enzyme (chymotrypsin, trypsin, elastase, etc.). The incubations were performed at 37° C. Fluorescence (320 nm excitation wavelength and 405 nm emission wavelength) was measured immediately after addition of the enzyme to the 96 well plate and also every minute for at least the next 30 minutes. The concentration of the enzyme was optimized to allow determination of slopes for the time course of initial fluorescence increase with and without the N-terminally acylated peptides or oligopeptides of the invention. The slopes were determined by linear regression of the linear part of the fluorescence trace (for example, the first 10 min of the reaction). Each assay was performed in duplicate and average of the two traces was included in the calculations. The relative effect of N-terminally acylated peptide or oligopeptides of the invention on enzymatic degradation of GLP-1 FRET substrate was obtained by comparison of the slopes achieved by the same concentration of the N-terminally acylated peptide or oligopeptides. The inhibition effect was also expressed as the concentration of the N-terminally acylated peptide or oligopeptide of the invention at which the slope of the fluorescence trace equals to 50% of uninhibited reaction (EC50). This was done by plotting the slopes achieved with different concentrations of the N-terminally acylated peptide or oligopeptides of the invention as a function of their concentrations and fitting the experimental results using, for example, sigmoidal logistic regression (2 parameters, Sigma Plot v 11). Inhibition constants for the interaction between the N-terminally acylated peptide or oligopeptides of the invention and proteolytic enzymes were also obtained by performing the assay described above with varying concentrations of the inhibitor and substrate and analyzing the results, for example, by double reciprocal transformation as known to the person skilled in the art and described for example in Hubalek, F. et al., J. Med. Chem. 47, 1760-1766 (2004).

The EC₅₀ was determined for the following compounds. EC₅₀±standard error is reported if the results of at least 3 independent measurements were available.

TABLE 1

| Compound from | EC₅₀ Chymotrypsin | EC₅₀ Trypsin |
|---|---|---|
| example # | (0.001 mg/ml) | (0.001 mg/ml) |
| 1 | 476 | >500 |
| 2 | 85 | 112 |
| 3 | 57 ± 2 | 74 |
| 5 | 105 | |
| 18 | 167 | |
| 19 | 121 | 38 |
| 20 | >500 | |
| 21 | >500 | |
| 22 | >500 | |
| 23 | 144 | 44 |
| 24 | 379 | |
| 25 | >500 | |
| 26 | 29 ± 2 | 413 |
| 27 | >500 | |
| 29 | 23 ± 2 | >500 |
| 32 | 29 ± 4 | 66 |
| 34 | 200 ± 26 | 317 |
| 35 | 182 | |
| 126 | 102 | >500 |
| 129 | 341 | 391 |
| 134 | 48 | 46 |
| 135 | 45 | 40 |
| 136 | 106 | 13 |
| 137 | >500 | |
| 138 | 154 | 39 |
| 139 | >500 | |
| 140 | 28 | 374 |
| 141 | 57 | 187 |
| 142 | 20 | 37 |
| 143 | 14 | 388 |
| 144 | 40 | 72 |
| 145 | 83 | 43 |
| 148 | 322 | |
| 149 | 48 | 57 |
| 150 | 13 | 22 |
| 151 | 9 | 13 |
| 152 | 293 | |
| 153 | 38 | 43 |
| 154 | 27 | 151 |
| 155 | 56 | 76 |
| 156 | 428 | >500 |
| 157 | 57 | 88 |
| 158 | 58 | 112 |
| 159 | 35 | 122 |
| 160 | 17 | 16 |
| 161 | 4 | 7 |
| 162 | 94 | 58 |
| 163 | 94 | 271 |
| 164 | 54 | 343 |
| 165 | 154 | 120 |
| 166 | 61 | 69 |
| 167 | >500 | |
| 168 | 377 | |
| 169 | 287 | |
| 170 | >500 | |
| 171 | 452 | |
| 172 | 132 | 401 |
| 173 | 51 | 93 |
| 174 | 211 | |
| 175 | 17 | 31 |
| 176 | 105 | 63 |
| 177 | 95 | 200 |
| 178 | 32 | 102 |
| 179 | 84 | 80 |
| 180 | >500 | |
| 181 | 300 | |
| 182 | 67 | 33 |
| 183 | 386 | |
| 184 | 69 | 38 |
| 185 | 73 | 44 |
| 186 | 56 | |
| 187 | 14 | 130 |
| 188 | 71 | 67 |
| 189 | 9 | 13 |
| 190 | >500 | |
| 191 | 136 | 177 |
| 192 | 34 | 303 |
| 193 | 90 | 169 |
| 194 | 19 | 84 |
| 195 | 17 | 7 |
| 196 | 6 | 75 |
| 197 | 2 | 16 |

The following compounds were tested (1 mM) as described above:

TABLE 2

| | Slope of the initial increase | Slope of the initial increase in |
|---|---|---|
| Compound | in fluorescence obtained | fluorescence obtained during |
| from | during incubation with | incubation with 0.001 mg/ml |
| example # | 0.001 mg/ml Chymotrypsin | Trypsin |
| 1 | 0.41 | 0.39 |
| 2 | 0.05 | 0.15 |
| 3 | 0.01 | 0.37 |
| 4 | 0.08 | 0.83 |
| 5 | 0.09 | 0.35 |
| 6 | 0.24 | 0.35 |
| 7 | 0.34 | 0.49 |
| 8 | 0.03 | 0.70 |
| 9 | 0.01 | 0.21 |
| 10 | 0.05 | 3.29 |
| 11 | 0.77 | 3.06 |
| 12 | 0.76 | 0.30 |
| 13 | 0.00 | 0.12 |
| 14 | 3.53 | 4.46 |
| 15 | 2.07 | 4.17 |
| 16 | 0.14 | 3.23 |
| 34 | 0.07 | 0.31 |
| 125 | 0.75 | 0.38 |
| 126 | 0.05 | 0.30 |
| 127 | 0.62 | 0.72 |
| 128 | 0.86 | 0.35 |
| 129 | 0.16 | 0.38 |
| 130 | 0.54 | 0.67 |
| 131 | 0.53 | 0.62 |
| 132 | 0.35 | 0.26 |
| 133 | 0.27 | 0.09 |

The following compounds were tested (0.1 mM) as described above with buffer containing 8% DMSO

TABLE 3

| | Slope of the initial increase | Slope of the initial increase in |
|---|---|---|
| Compound | in fluorescence obtained | fluorescence obtained during |
| from | during incubation with | incubation with 0.001 mg/ml |
| example # | 0.001 mg/ml Chymotrypsin | Trypsin |
| 36 | 0.16 | 1.47 |
| 37 | 0.66 | 1.78 |
| 38 | 0.02 | 1.66 |
| 39 | 0.27 | 1.84 |
| 40 | 0.41 | 1.31 |
| 41 | 0.84 | 1.96 |
| 42 | 0.00 | 0.89 |
| 43 | 0.21 | 1.36 |
| 44 | 0.00 | 0.96 |
| 45 | 0.89 | 1.84 |
| 46 | 0.09 | 1.76 |
| 47 | 0.48 | 1.77 |
| 48 | 0.07 | 1.55 |
| 49 | 0.57 | 1.96 |
| 50 | 0.09 | 1.48 |
| 51 | 0.41 | 1.66 |
| 52 | 0.01 | 1.68 |
| 53 | 0.16 | 1.80 |
| 54 | 0.95 | 1.95 |
| 55 | 0.42 | 1.88 |
| 56 | 0.59 | 1.92 |
| 57 | 1.65 | 1.59 |
| 58 | 0.28 | 1.98 |
| 59 | 1.79 | 1.72 |
| 60 | 0.91 | 1.73 |
| 61 | 2.20 | 2.07 |
| 62 | 1.35 | 1.66 |
| 63 | 1.89 | 1.90 |
| 64 | 1.23 | 2.23 |
| 65 | 2.04 | 1.60 |
| 66 | 0.18 | 1.98 |
| 67 | 0.94 | 1.89 |
| 68 | 0.60 | 1.73 |
| 69 | 0.43 | 1.83 |
| 70 | 1.50 | 1.45 |
| 71 | 0.91 | 1.76 |
| 72 | 1.18 | 1.52 |
| 73 | 0.08 | 1.67 |
| 74 | 1.66 | 2.27 |
| 75 | 1.77 | 1.81 |
| 76 | 0.85 | 2.22 |
| 77 | 1.56 | 1.55 |
| 78 | 1.03 | 1.87 |
| 79 | 0.51 | 1.93 |
| 80 | 0.10 | 1.83 |
| 81 | 0.40 | 1.90 |
| 82 | 0.24 | 1.50 |
| 83 | 0.32 | 1.64 |
| 84 | 0.14 | 1.43 |
| 85 | 1.81 | 1.92 |
| 86 | 0.33 | 1.89 |
| 87 | 0.48 | 1.91 |
| 88 | 0.11 | 0.80 |
| 89 | 0.80 | 1.86 |
| 90 | 0.35 | 1.25 |
| 91 | 0.00 | 1.06 |
| 92 | 0.23 | 1.44 |
| 93 | 0.14 | 1.90 |
| 94 | 1.83 | 1.90 |
| 95 | 0.10 | 1.82 |
| 96 | 1.63 | 1.78 |
| 97 | 0.55 | 1.31 |
| 98 | 1.39 | 1.53 |
| 99 | 0.35 | 1.52 |
| 100 | 1.75 | 1.69 |
| 102 | 0.14 | 1.66 |
| 103 | 1.13 | 1.78 |
| 104 | 0.16 | 1.63 |
| 105 | 1.31 | 1.56 |
| 106 | 1.72 | 1.75 |
| 107 | 0.89 | 1.99 |
| 108 | 0.59 | 1.92 |
| 109 | 0.05 | 0.73 |
| 110 | 0.35 | 1.77 |
| 111 | 0.31 | 1.93 |
| 112 | 1.44 | 2.35 |
| 113 | 0.18 | 2.00 |
| 114 | 2.01 | 1.99 |
| 115 | 2.04 | 1.73 |
| 116 | 1.48 | 1.75 |
| 117 | 1.82 | 1.66 |
| 118 | 1.94 | 1.84 |
| 119 | 0.70 | 2.13 |
| 120 | 0.17 | 1.08 |
| 121 | 0.34 | 1.35 |
| 122 | 0.26 | 1.68 |
| 123 | 0.82 | 2.11 |
| 124 | 0.12 | 0.63 |

### Example 199

### Inhibition of Enzymatic Degradation of a Model Insulin

The use of Förster resonance energy transfer (FRET), also known as fluorescence resonance energy transfer, substrates to monitor activity of proteolytic enzymes is known in the field (for example Anjuere, F. et al. (1993). Biochem J 291 (Pt 3), 869-73).

Model insulin analogue is designed as Förster resonance energy transfer (FRET) substrate by incorporation of MCA group at the N-terminus of the A-chain as the donor chromophore and DNP group attached to B29 lysine via hexanoyl linker as the acceptor chromophore to obtain the insulin FRET substrate, e.g. A1N-7-methoxycoumarin-4-acetyl B29N(eps)-2,4-dinitrophenylamino-hexanoyl A14E B25H desB30 human insulin.

An assay following the increase in fluorescence as a function of time is established in 96 well format using Varioskan Flash Multimode Meter (Thermo Scientific). Each well contained 70 μl of Dulbecco's phosphate buffer saline (Invitrogen catalogue #14190-094), 10 μl of 100 μM insulin FRET substrate, 10 μl of N-terminally acylated peptide or oligopeptide of the invention in varying concentration and 10 μl of a stock solution of an enzyme (chymotrypsin, trypsin or elastase). The incubations are performed at 37° C. Fluorescence (320 nm excitation wavelength and 405 nm emission wavelength) is measured immediately after addition of the enzyme to the 96 well plate and also every minute for the next 80 minutes. The concentration of the enzyme is optimized to allow determination of slopes for the time course of initial fluorescence increase with and without the N-terminally acylated peptides or oligopeptides of the invention. The slopes are determined by linear regression of the linear part of the fluorescence trace (for example, the first 10 min of the reaction). Each assay is typically performed in duplicate and average of the two traces is included in the calculations. The relative effect of N-terminally acylated peptide or oligopeptides of the invention on enzymatic degradation of insulin FRET substrate is obtained by comparison of the slopes achieved by the same concentration of the N-terminally acylated peptide or oligopeptides. The inhibition effect is also expressed as the concentration of the N-terminally acylated peptide or oligopeptide of the invention at which the slope of the fluorescence trace equals to 50% of uninhibited reaction (EC50). This is done by plotting the slopes achieved with different concentrations of the N-terminally acylated peptide or oligopeptides of the invention as a function of their concentrations and fitting the experimental results using, for example, sigmoidal logistic regression (2 parameters, Sigma Plot v 11). Inhibition constants for the interaction between the N-terminally acylated peptide or oligopeptides of the invention and proteolytic enzymes are also obtained by performing the assay described above with varying concentrations of the inhibitor and substrate and analyzing the results, for example, by double reciprocal transformation as known to the person skilled in the art and described for example in Hubalek, F. et al J. Med. Chem. 47, 1760-1766 (2004).

### Example 200

### Inhibition of GI Juice Degradation of a Model Insulin and GLP-1

96 well plates were coated by incubating with 0.4% Bovine serum albumin (BSA) solution for minimum of 60 min. To each well, 210 μl of buffer (Hank's balanced salt solution-4-(2-hydroxyethyl)-1-piperazineethanesulfonic acid (HBSS-HEPES) buffer) with 0.005% Tween 20 and 0.001% BSA, pH 6.5 precipitated with 3 vol. cold 96% ethanol (EtOH) w. 1% TFA), 30 μl of a substrate (100 μM insulin analogue or GLP-1 analogue in buffer) and 30 μl of N-terminally acylated peptide or oligopeptide of the invention (10 mM) were added. The plates were pre-incubated (before adding GI juice) for 60 min at 37° C. After addition of 30 μl of GI juice (10-times diluted in buffer), the plates were incubated for 60 min/37° C. on shaker. Samples (40 μl) were taken at 0, 5, 10, 20, 30 and 60 min, stopped with 3 vol. cold 96% EtOH w. 1% TFA and spun down in plates (4500 rpm for 10 min). Samples were diluted 5 times with the buffer prior to LC-MS analysis. Standard samples (0.1, 0.5, 1.0, 5.0, 10.0 μM) were prepared and treated as the samples. Standard curve was analysed both at the beginning and the end of the sequence. Two replicates of each tested conditions were included. Ion suppression was assessed by analyzing a standard at 1 μM and at 10 μM with 1 mM inhibitor present. Intact insulin or GLP-1 analoues were determined at each sample. The results were plotted against the incubation time. Half-lives of the insulin or GLP-1 analogues were determined by nonlinear regression of the results using, for example Graph Pad Prism. The half-lives were expressed relative to the half-live of the insulin or GLP-1 analogue without inhibitor present by dividing the half-lives obtained in the presence of inhibitor with those obtained in the absence of inhibitors. GI juice was prepared from male Sprague Dawley rats (200-250 g) by excising approximately 20 cm piece of mid jejunum and rinsing the inside with 2.5 ml 0.9% sodium chloride solution. The sodium chloride solution was collected in a centrifuge tube, pooled from all rats (20) and centrifuged at 4500 rpm./10 min/4° C. The supernatant was aliquoted in tubes and stored at −80° C.

A14E, B25H, B29K(N(eps)Octadecanedioyl-γGlu-OEG-OEG), desB30 human insulin and N-epsilon26-[2-(2-{2-[2-(2-{2-[(S)-4-Carboxy-4-(17-carboxyheptadecanoylamino)-butyrylamino]ethoxy}ethoxy)acetylamino]ethoxy}ethoxy)acetyl][Aib8,Arg34]GLP-1-(7-37) were used as standards in this assay.

For experiments repeated more than two times, standard deviation is given

TABLE 4

| | | | | Half Life fold |
|---|---|---|---|---|
| | | | | increase (over |
| | | Half Life | | solution w/o N- |
| Compound | | of GLP- | Half Life of | terminally |
| from | Conc. | 1 analogue^{#} | insulin | acylated |
| example # | (mM) | (min) | analogue^{%} (min) | (oligo)peptide) |
| 1 | 1 mM | | 13.0 | 5.0 |
| 2 | 1 mM | | 14.3 | 6.8 |
| 3 | 1 mM | | 10 | 4.5 |
| 4 | 1 mM | | 5.0 | 1.3 |
| 5 | 1 mM | | 23.5 | 2.9 |
| 6 | 1 mM | | 3.2 | 1.2 |
| 7 | 1 mM | | 4.6 | 1.8 |
| 8 | 1 mM | | 9.5 | 2.4 |
| 9 | 1 mM | | 8.9 | 4.2 |
| 10 | 1 mM | | 7.6 | 2.9 |
| 11 | 1 mM | | 5.9 | 2.5 |
| 12 | 1 mM | | 8.1 | 3.1 |
| 13 | 1 mM | | 7.2 | 3.4 |
| 14 | 1 mM | | 2.1 | 0.9 |
| 15 | 1 mM | | 4.3 | 1.8 |
| 16 | 1 mM | | 9.0 | 2.0 |
| 17 | 1 mM | | 4.0 | 2.5 |
| 21 | 1 mM | | 4.6 | 2.3 |
| 22 | 1 mM | | 11.7 | 5.9 |
| 23 | 1 mM | | 34.8 | 17.4 |
| 24 | 1 mM | | 6.7 | 3.4 |
| 25 | 1 mM | | 4.1 | 2.1 |
| 26 | 1 mM | | 14.3 | 8.9 |
| 27 | 1 mM | | 4.4 | 2.2 |
| 28 | 1 mM | | 10.7 | 5.1 |
| 29 | 1 mM | 2.7 | 15.1 | 9.4 |
| 30 | 1 mM | | 5.9 | 2.8 |
| 31 | 1 mM | | 4.2 | 2.0 |
| 34 | 1 mM | | 10.8 | 5.7 |
| 35 | 1 mM | | 9.8 | 3.5 |
| 125 | 1 mM | | 11.0 | 2.4 |
| 126 | 1 mM | | 6.8 | 3.2 |
| 127 | 1 mM | | 5.0 | 2.1 |
| 128 | 1 mM | | 2.5 | 0.6 |
| 129 | 1 mM | | 13.1 | 5.0 |
| 130 | 1 mM | | 5.7 | 1.2 |
| 131 | 1 mM | | 7.6 | 1.7 |
| 132 | 1 mM | | 2.7 | 1.0 |
| 133 | 1 mM | | 3.8 | 1.7 |
| 134 | 1 mM | 1.8 | 8.7 | 3.2 |
| 135 | 1 mM | 0.9 | 9.2 | 3.8 |
| 136 | 1 mM | | 10.0 | 4.2 |
| 140 | 1 mM | 0.7 | 9.1 | 3.8 |
| 141 | 1 mM | | 12.3 | 4.4 |
| 142 | 1 mM | | 20.1 | 8.4 |
| 143 | 1 mM | 2.0 | 7.9 | 3.3 |
| 144 | 1 mM | | 14.7 | 5.4 |
| 145 | 1 mM | | 11.2 | 4.1 |
| 148 | 1 mM | | 3.3 | 1.4 |
| 149 | 1 mM | 1.0 | 9.9 | 3.5 |
| 150 | 1 mM | | 11.1 | 4.3 |
| 151 | 1 mM | | 10.8 | 4.2 |
| 153 | 1 mM | | 16.7 | 6.0 |
| 154 | 1 mM | | 27.6 | 9.9 |
| 155 | 1 mM | | 6.1 | 2.9 |
| 156 | 1 mM | 1.1 | 5.0 | 2.4 |
| 157 | 1 mM | | 6.6 | 3.1 |
| 160 | 1 mM | | 11.3 | 5.9 |
| 161 | 1 mM | | 12.2 | 6.6 |
| 166 | 1 mM | 0.8 | 11.4 | 7.6 |
| 173 | 1 mM | | 12.2 | 5.7 |
| 175 | 1 mM | | 13.7 | 7.2 |
| 178 | 1 mM | 1.1 | 40.6 | 22.5 |
| 183 | 1 mM | | 6.2 | 1.3 |
| 184 | 1 mM | | 18.7 | 4.0 |
| 185 | 1 mM | 0.9 | 40.4 | 21.3 |
| 186 | 1 mM | | 20.3 | 10.7 |
| 187 | 1 mM | | 18.1 | 9.5 |
| 38 | 1 mM* | | 472 | 78.7 |
| 42 | 1 mM* | | 45 | 7.5 |
| 44 | 1 mM* | | 835 | 139 |
| 52 | 1 mM* | | 84 | 14 |
| 91 | 1 mM* | | 455 | 75.8 |
| 194 | 1 mM | 0.8 | 15.6 | 6.7 |
| 196 | 1 mM | 2.6 | 16.1 | 6.9 |
| 197 | 1 mM | 3.9 | 20.0 | 8.6 |
| No | | | 2.9 ± 1.9 | 1.0 |
| Inhibitor | | | | |
| Soya bean | 0.1% | | 505.3 ± 309.2 | |
| trypsin | | | | |
| inhibitor | | | | |

^{%}insulin analogue = A14E, B25H, B29K(N(eps)Octadecanedioyl-γGlu-OEG-OEG), desB30 human insulin
^{#}GLP-1 analogue = N-epsilon26-[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl]-[Aib8,Arg34]GLP-1-(7-37)
*assay was performed in 8% DMSO in the buffer described above

For experiments repeated more than 2-times, standard deviation is given

Degradation of the N-terminally fatty acid modified peptide or oligopeptide of the invention themselves in jejunum extract from rat (GI juice) and determination of half-lives, was measured as described above (example 200). The results showed that the N-terminally fatty acid modified peptides or oligopeptides of the invention containing all-D amino acids are stable in GI juice (half-lives >500 min), and the N-terminally fatty acid modified peptide or oligopeptide of the invention containing all-L amino acids have half-lives around 4-6 min.

TABLE 5
Stability of N-terminally acylated peptide or oligopeptides of the invention
in GI juice

| Compound from example # | t 1/2 in rat GI juice (min) |
|---|---|
| 34 | 3.5 |
| 1 | >1000 |
| 2 | 610 |
| 23 | 4.4 |
| 148 | >1000 |
| 178 | 4.5 |

### Example 201

### Inhibition of Insulin Degradation by Duodenum Lumen Enzymes

Degradation using duodenum lumen enzymes (prepared by filtration of duodenum lumen content) from SPD rats.

Each HPLC vial contained Dulbecco's phosphate buffer saline (DPBS, Invitrogen catalogue #14190-094), A14E, B25H, B29K(N(eps)Octadecanedioyl-γGlu-OEG-OEG), desB30 human insulin, N-terminally acylated peptide or oligopeptide of the invention and enzyme (chymotrypsin, trypsin, elastase or duodenum lumen enzymes). Total volume was 150 μl and the concentrations of insulin and the N-terminally acylated peptide or oligopeptide of the invention were varied to allow determination of EC50 and Kᵢ, for example 15 μl of 150 μM A14E, B25H, B29K(N(eps)Octadecanedioyl-γGlu-OEG-OEG), desB30 human insulin, 1 μl of 10 mM oligoeptide, 114 μl of DPBS and 20 μl of 0.1 mg/ml chymotrypsin.

The assay was performed in an HPLC autosampler equilibrated at 37° C., at specified time points, aliquots were injected directly onto an HPLC column and the amount of the intact A14E, B25H, B29K(N(eps)Octadecanedioyl-γGlu-OEG-OEG), desB30 human insulin was determined. Degradation half life was determined by exponential fitting of the data (for example, single exponential decay, 2 parameters, Sigma Plot version 11, Systat Software) and normalized to half time determined for the reference insulins, or human insulin in each assay. The inhibition effect was also expressed as the concentration of the N-terminally acylated peptide or oligopeptide of the invention at which the half-life of the insulin equaled 50% of uninhibited reaction (EC50). This was done by plotting the half life achieved with different concentrations of the N-terminally acylated peptide or oligopeptides of the invention as a function of their concentrations and fitting the experimental results using, for example, sigmoidal logistic regression (2 parameters, Sigma Plot v 11). Inhibition constants for the interaction between the N-terminally acylated peptide or oligopeptides of the invention and proteolytic enzymes were also obtained by performing the assay described above with varying concentrations of the inhibitor and substrate and analyzing the results, for example, by double reciprocal transformation as known to the person skilled in the art and described for example in Hubalek, F. et al., J. Med. Chem. 47, 1760-1766 (2004). Other algorithms known to the person skilled in the art may also be used to determine inhibition constants from the results.

TABLE 6

| Compound from | |
|---|---|
| example# | Kᵢ (M) |
| 34 | 1.5 × 10⁻⁵ |

### Example 202

### Inhibition of Enzymatic Degradation of a Chromogenic Substrate

The use of chromogenic substrates to monitor activity of proteolytic enzymes is known in the field (for example DelMar, E. G., et al., Anal. Biochem., 99, 316-320, (1979)). For example, N-succinyl-Ala-Ala-Pro-Phe-p-Nitroanilide is commonly used substrate for measuring chymotrypsin activity. Enzymatic cleavage of 4-nitroanilide substrates yields 4-nitroaniline (yellow color under alkaline conditions).

An assay following the increase in absorbance at 395 nm as a function of time was established in 96 well format using Varioskan Flash Multimode Meter (Thermo Scientific). Each well contained 70 μl of Dulbecco's phosphate buffer saline (Invitrogen catalogue #14190-094), 10 μl of N-succinyl-Ala-Ala-Pro-Phe-p-Nitroanilide (Sigma cat# S 7388) in DMSO (different concentrations were used in order to obtain the inhibition constant), 10 μl of N-terminally acylated peptide or oligopeptide of the invention in varying concentration and 10 μl of a stock solution of an enzyme (chymotrypsin, trypsin, elastase, etc.). The incubations were performed at 37° C. Absorbance at 395 nm was measured immediately after addition of the enzyme to the 96 well plate and also every minute for the next 80 minutes. The concentration of the enzyme was optimized to allow determination of slopes for the time course of initial absorbance increase with and without added inhibitors. The slopes were determined by linear regression of the linear part of the fluorescence trace (for example, the first 10 min of the reaction). Each assay was performed in duplicate and average of the two traces was included in the calculations. The relative effect of N-terminally acylated peptide or oligopeptides of the invention on enzymatic degradation of N-succinyl-Ala-Ala-Pro-Phe-p-Nitroanilide was obtained by comparison of the slopes achieved by the same concentration of the N-terminally acylated peptide or oligopeptides. The inhibition effect was also expressed as the concentration of the N-terminally acylated peptide or oligopeptide of the invention at which the slope of the absorbance trace equals to 50% of uninhibited reaction (EC50). This was done by plotting the slopes achieved with different concentrations of the N-terminally acylated peptide or oligopeptides of the invention as a function of their concentrations and fitting the experimental results using, for example, sigmoidal logistic regression (2 parameters, Sigma Plot v 11). Inhibition constants for the interaction between the N-terminally acylated peptide or oligopeptides of the invention and proteolytic enzymes were also obtained by performing the assay described above with varying concentrations of the inhibitor and substrate and analyzing the results, for example, by double reciprocal transformation as known to the person skilled in the art and described for example in Hubalek, F. et al., J. Med. Chem. 47, 1760-1766 (2004).

TABLE 7

| Compound from | |
|---|---|
| example# | Kᵢ (M) |
| 33 | 8.5 × 10⁻⁴ |
| 34 | 1.3 × 10⁻⁴ |

### Example 203

### Hydrophobicity of the N-Terminally Modified Oligopeptides of the Invention

The retention time (RT) during reverse phase HPLC was taken as a measure of hydrophobicity of an N-terminally modified peptide or oligopeptide of the invention, where the connection is: The longer the RT, the more hydrophobic N-terminally modified peptide or oligopeptide. The following running conditions were applied during HPLC analysis:

Column: Acquity CSH 1.7 μm C18 1×150 mm

Buffer A: 0.2 M Na₂SO₄, 0.02M Na₂HPO₄, 0.02M NaH₂PO₄, 10% (v/v) CH₃CN, pH 7.2

Buffer B: 70% (v/v) aq. CH₃CN

Injection volume: 1 μl

Detection: UV at 220 nm

Temperature: 40° C.

Run time: 20 minutes

Gradient:

| Time (min) | Flow rate (ml/min) | % A | % B | Curve |
|---|---|---|---|---|
| initial | 0.1 | 100 | 0 | |
| 1.0 | 0.1 | 50 | 50 | 11 |
| 1.0 | 0.1 | 50 | 50 | 11 |
| 17.0 | 0.1 | 0 | 100 | 6 |
| 18 | 0.1 | 100 | 0 | 6 |

Dead volume of the system was determined to be 125 μl as examined by analyzing a solution of 10 mM NaNO₃ that eluted with retention time of 1.25 min.

TABLE 8

| Compound described in example # | RT (min) |
|---|---|
| 1 | 6.91 |
| 2 | 9.74 |
| 3 | 9.74 |
| 4 | 6.89 |
| 5 | 9.74 |
| 6 | 5.96 |
| 7 | 8.06 |
| 8 | 6.13 |
| 9 | 8.73 |
| 10 | 6.04 |
| 11 | 1.22 |
| 12 | 4.04 |
| 13 | 5.19 |
| 14 | 3.08 |
| 15 | 3.13 |
| 16 | 3.11 |
| 17 | 5.83 |
| 18 | 6.59 |
| 19 | 6.57 |
| 20 | 3.79 |
| 21 | 3.66 |
| 22 | 4.09 |
| 23 | 5.29 |
| 24 | 6.88 |
| 25 | 3.66 |
| 26 | 7.03 |
| 33 | 4.93 |
| 34 | 6.94 |
| 36 | 5.33 |
| 37 | 5.41 |
| 38 | 4.94 |
| 39 | 5.08 |
| 40 | 7.77 |
| 41 | 7.76 |
| 42 | 7.87 |
| 43 | 7.86 |
| 44 | 4.46 |
| 45 | 4.62 |
| 46 | 7.06 |
| 47 | 7.09 |
| 48 | 6.2 |
| 49 | 6.2 |
| 50 | 5.16 |
| 51 | 5.2 |
| 52 | 5.47 |
| 53 | 5.52 |
| 54 | 7.08 |
| 55 | 7.01 |
| 56 | 5.74 |
| 57 | 5.73 |
| 58 | 4.87 |
| 59 | 7.88 |
| 60 | 4.33 |
| 61 | 8.28 |
| 62 | 4.36 |
| 63 | 5.46 |
| 64 | 5.33 |
| 65 | 3.12 |
| 66 | 7.18 |
| 67 | 4.7 |
| 68 | 6.68 |
| 69 | 5.79 |
| 70 | 5.43 |
| 71 | 5.61 |
| 72 | 6.44 |
| 73 | 5.34 |
| 74 | 5.27 |
| 75 | 4.2 |
| 76 | 5.26 |
| 77 | 4.17 |
| 78 | 5.59 |
| 79 | 5.29 |
| 80 | 7.24 |
| 81 | 7.38 |
| 82 | 5.07 |
| 83 | 6.88 |
| 84 | 7.77 |
| 85 | 6.1 |
| 86 | 5.64 |
| 87 | 5.76 |
| 88 | 7.88 |
| 89 | 6.23 |
| 90 | 6.6 |
| 91 | 4.63 |
| 92 | 4.84 |
| 93 | 4.84 |
| 94 | 4.77 |
| 95 | 3.92 |
| 96 | 3.86 |
| 97 | 3.79 |
| 98 | 3.79 |
| 99 | 3.41 |
| 100 | 3.9 |
| 102 | 4.82 |
| 103 | 4.92 |
| 104 | 3.83 |
| 105 | 3.91 |
| 106 | 6.01 |
| 107 | 5.58 |
| 108 | 5.72 |
| 109 | 8.24 |
| 110 | 6.24 |
| 111 | 7 |
| 112 | 6.6 |
| 113 | 5.29 |
| 114 | 5.07 |
| 115 | 3.91 |
| 116 | 5.08 |
| 117 | 3.91 |
| 118 | 5.35 |
| 119 | 5.12 |
| 120 | 7.82 |
| 121 | 7.77 |
| 122 | 5.17 |
| 123 | 7.23 |
| 124 | 8.19 |
| 125 | 3.95 |
| 126 | 5.02 |
| 127 | 3.67 |
| 128 | 3.08 |
| 129 | 6.34 |
| 130 | 6.21 |
| 131 | 6.21 |
| 132 | 3.62 |
| 133 | 3.62 |
| 134 | 7 |
| 135 | 7.5 |
| 136 | 8.73 |
| 137 | 7.7 |
| 138 | 8.16 |
| 139 | 4.95 |

### Example 204

### Rat Pharmacokinecics, Rat PK Following Intraintestinal Injection

Anaesthetized rats were dosed intraintestinally (into jejunum) with reference compounds and N-terminally acylated peptide or oligopeptides of the invention. Plasma concentrations of the employed compounds as well as changes in blood glucose were measured at specified intervals for 4 hours or more post-dosing. Pharmacokinetic parameters were subsequently calculated using WinNonLin Professional (Pharsight Inc., Mountain View, Calif., USA).

Male Sprague-Dawley rats (Taconic), weighing 250-300 g, fasted for ˜18 h were anesthetized using Hypnorm-Dormicum s.c. (0.079 mg/ml fentanyl citrate, 2.5 mg/ml fluanisone and 1.25 mg/ml midazolam) 2 ml/kg as a priming dose (to timepoint −60 min prior to test substance dosing), 1 ml/kg after 20 min followed by 1 ml/kg every 40 min.

The compositions for the intraintestinal injection model were prepared for example according to the following composition (in weight %):

600 nmol/g Reference insulin compound

3% N-terminally acylated peptide or oligopeptide of the invention

15% Propylene glycol

51.6% diglycerol caprilate

30% Tween 20

The anesthetized rat was placed on a homeothermic blanket stabilized at 37° C. A 20 cm polyethylene catheter mounted a 1-ml syringe was filled with insulin composition or vehicle. A 4-5 cm midline incision was made in the abdominal wall. The catheter was gently inserted into mid-jejunum ˜50 cm from the caecum by penetration of the intestinal wall. If intestinal content was present, the application site was moved ±10 cm. The catheter tip was placed approx. 2 cm inside the lumen of the intestinal segment and fixed without the use of ligatures. The intestines were carefully replaced in the abdominal cavity and the abdominal wall and skin were closed with autoclips in each layer. At time 0, the rats were dosed via the catheter, 0.4 ml/kg of test compound or vehicle.

Blood samples for the determination of whole blood glucose concentrations were collected in heparinised 10 μl capillary tubes by puncture of the capillary vessels in the tail tip. Blood glucose concentrations were measured after dilution in 500 μl analysis buffer by the glucose oxidase method using a Biosen autoanalyzer (EKF Diagnostic Gmbh, Germany). Mean blood glucose concentration courses (mean±SEM) were made for each compound.

Samples were collected for determination of the plasma insulin concentration. 100 μl blood samples were drawn into chilled tubes containing EDTA. The samples were kept on ice until centrifuged (7000 rpm, 4° C., 5 min), plasma was pipetted into Micronic tubes and then frozen at 20° C. until assay. Plasma concentrations of the insulin analogues were measured in a immunoassay.

Blood samples were drawn at t=−10 (for blood glucose only), at t=−1 (just before dosing) and at specified intervals for 4 hours or more post-dosing.

TABLE 9

| Compound from Example# | Bioavailability (%) | Composition |
|---|---|---|
| 29 | 8.6 ± 7.8 | 1 |
| 126 | 1.5 ± 1.1 | 1 |
| 159 | 0.9 ± 0.8 | 1 |
| 183 | 6.2 ± 4.8 | 1 |
| 184 | 5.5 ± 2.3 | 1 |
| 187 | 4.4 ± 3.7 | 1 |
| 189 | 12 ± 5.8 | 1 |
| 5 | 1.9 ± 1.1 | 2 |
| 23 | 0.9 ± 0.6 | 2 |
| 148 | 1 ± 0.8 | 2 |
| 151 | 0.9 ± 1 | 2 |
| 155 | 1.6 ± 1.5 | 2 |
| 1 | 4.5 ± 2.7 | 3 |
| 2 | 19 ± 7 | 3 |
| 3 | 9.7 ± 3.3 | 3 |
| 5 | 13 ± 8.2 | 3 |
| 23 | 18 ± 8.3 | 3 |
| 26 | 13 ± 9.2 | 3 |
| 32 | 9.2 ± 4.6 | 3 |
| 34 | 10.6 ± 6.2 | 3 |
| 136 | 7.1 ± 4.9 | 3 |
| 142 | 7.5 ± 3.2 | 3 |
| 143 | 9.2 ± 6.3 | 3 |
| 144 | 7.3 ± 5.6 | 3 |
| 145 | 4.8 ± 2.8 | 3 |
| 148 | 13 ± 4.4 | 3 |
| 149 | 3.9 ± 0.9 | 3 |
| 150 | 5.3 ± 3.9 | 3 |
| 151 | 9.1 ± 5.8 | 3 |
| 154 | 7 ± 4.2 | 3 |
| 155 | 11 ± 11 | 3 |
| 159 | 10 ± 6 | 3 |
| 175 | 6.4 ± 4.3 | 3 |
| 178 | 2.5 ± 1.9 | 3 |
| 181 | 6.3 ± 4.5 | 3 |
| 182 | 8.7 ± 0 | 3 |
| 183 | 5.6 ± 4.1 | 3 |
| 184 | 6.5 ± 5.1 | 3 |

Dose: 60 nmol/kg of insulin

Composition 1:

0.15 mM Reference insulin compound

0.1 M N-terminally acylated peptide or oligopeptide of the invention

5 mM phosphate buffer pH=8

Composition 2:

0.15 mM Reference insuln compound

10 mg/ml N-terminally acylated peptide or oligopeptide of the invention

5 mM phosphate buffer pH=8

Composition 3:

600 nmol/g Reference insuln compound

3% N-terminally acylated peptide or oligopeptide of the invention

15% Propylene glycol

51.6% diglycerol caprylate

30% Tween 20

### Example 205

### Transepithelial Transport in Caco-2 Cell Monolayers

Cell Culturing

Caco-2 cells were obtained from the American Type Culture Collection (Manassas, Va.). Cells were seeded in culturing flasks and passaged in Dulbecco's Modified Eagle' medium supplemented with 10% fetal bovine serum, 1% penicillin/streptomycin (100 U/ml and 100 μg/ml, respectively), 1% L-glutamine and 1% nonessential amino acids. Caco-2 cells were seeded onto tissue culture treated polycarbonate filters in 12-well Transwell plates (1.13 cm2, 0.4 μm pore size) at a density of 10⁵ cells/well. Monolayers were grown in an atmosphere of 5% CO₂-95% O₂ at 37° C. Growth media were replaced every other day. The experiment was performed on day 10-14 after seeding of Caco-2 cells.

Transepithelial Transport

The amount of compound transported from the donor chamber (apical side) to the receiver chamber (basolateral side) was measured. The transport study was initiated by adding 400 μl solution (100 μM of A14E, B25H, B29K(N(eps)Octadecanedioyl-γGlu-OEG-OEG), desB30 human insulin analogue, 100 μM of A14E, B25H, B29K(N(eps)Octadecanedioyl-γGlu-OEG-OEG), desB30 human insulin analogue and 0.5 mM N-terminally acylated peptide or oligopeptide of the invention) and 0.4 μCi/μl [3H]manntiol in transport buffer to the donor chamber and 1000 μl transport buffer to the receiver chamber, alternatively 400 μl solution (100 μM of N-epsilon26-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Aib8,Arg34]GLP-1-(7-37), 100 μM of N-epsilon26-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Aib8,Arg34]GLP-1-(7-37) and 0.5 mM N-terminally acylated peptide or oligopeptide of the invention) and 0.4 μCi/μl [3H]manntiol in transport buffer to the donor chamber and 1000 μl transport buffer to the receiver chamber. The transport buffer consisted of Hank's balanced saline solution containing 10 mM HEPES, 0.1% adjusted to pH 7.4 after addition of compounds. The transport of [³H]mannitol, a marker for paracellular transport, was measured to verify the integrity of the epithelium.

Before the experiment, the Caco-2 cells were equilibrated for 60 min with transport buffer on both sides of the epithelium. Buffer was then removed and the experiment initiated. Donor samples (20 μl) were taken at 0 min and at the end of the experiment. Receiver samples (200 μl) were taken every 15 min. The study was performed in an atmosphere of 5% CO₂-95% O₂ at 37° C. on a shaking plate (30 rpm).

In all samples with A14E, B25H, B29K(N(eps)Octadecanedioyl-γGlu-OEG-OEG), desB30 human insulin analogue and mannitol, alternatively N-epsilon26-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl][Aib8,Arg34]GLP-1-(7-37) and mannitol, the concentration was determined using a LOCI assay and scintillation counter, respectively.

Before and during the experiment the transepithelial electrical resistance (TEER) of the cell monolayers was monitored. In selected experiments, the transport buffer were changed to culturing medium after end of experiment and the TEER measured 24 h after experiment. The TEER was measured with EVOM™ Epithelial Voltohmmeter connected to Chopsticks.

Caco-2 permeability in the presence of the N-terminally acylated peptide or oligopeptides of the invention:

TABLE 10

| | Insulin* | | GLP-1^{#} absorption | |
|---|---|---|---|---|
| | absorption enhancement | | enhancement | |
| | in the presence | | in the presence | |
| | of N-terminally acylated | | of N-terminally acylated | |
| | peptide or oligopeptide | | peptide or oligopeptide | |
| | of the invention | | of the invention | |
| Compound | Papp | Papp | Papp | Papp |
| from example # | relative | (×10E−8 cm/s) | relative | (×10E−8 cm/s) |
| 1 | 1.9 | 2.5 | 1.9 | 1.0 |
| 2 | 11.0 | 24.8 | 82.2 | 68.6 |
| 3 | 8.0 | 9.3 | 3.8 | 2.6 |
| 5 | 17.0 | 38.2 | 6.7 | 5.6 |
| 8 | 1.4 | 3.0 | 1.9 | 1.6 |
| 9 | 8.4 | 18.9 | 83.2 | 69.5 |
| 11 | 2.2 | 1.2 | 1.0 | .5 |
| 13 | 2.3 | 5.2 | 1.4 | 1.2 |
| 14 | 1.2 | .6 | .6 | .3 |
| 15 | 1.3 | .7 | .6 | .3 |
| 16 | 1.6 | .8 | .7 | .4 |
| 17 | 2.0 | 1.1 | .8 | .4 |
| 22 | .7 | .7 | 1.3 | .5 |
| 23 | 2.9 | 2.7 | 3.5 | 1.4 |
| 29 | 22.0 | 11.7 | 10.4 | 5.2 |
| 32 | 10.5 | 6.7 | 4.6 | 1.9 |
| 33 | 1.5 | .8 | 1.0 | .5 |
| 34 | 2.2 | 1.5 | 2.5 | .9 |
| 126 | 1.3 | 2.9 | 95.4 | 79.6 |
| 129 | 1.0 | .6 | 1.2 | .3 |
| 133 | .8 | 1.7 | 1.3 | 1.1 |
| 134 | 3.6 | 2.2 | 2.5 | .6 |
| 135 | 5.7 | 3.5 | 4.2 | 1.0 |
| 142 | 2.9 | 1.8 | 2.7 | .7 |
| 143 | 2.6 | 1.6 | 2.7 | .7 |
| 144 | 5.7 | 3.5 | 4.9 | 1.2 |
| 145 | 1.0 | .6 | 2.0 | .5 |
| 148 | 2.7 | 1.7 | 2.7 | .7 |
| 149 | 9.4 | 8.5 | 10.6 | 4.1 |
| 150 | 16.3 | 14.8 | 19.0 | 7.3 |
| 151 | 19.2 | 17.4 | 17.5 | 6.8 |
| 155 | 11.7 | 10.6 | 9.3 | 3.6 |
| 156 | 4.5 | 4.1 | 2.3 | .9 |
| 159 | 4.3 | 2.4 | 4.2 | 1.1 |
| 160 | 10.8 | 6.1 | 6.6 | 1.8 |
| 161 | 14.7 | 8.3 | 6.8 | 1.8 |
| 173 | 5.9 | 3.3 | 4.4 | 1.2 |
| 175 | 16.5 | 9.3 | | |
| 178 | 4.5 | 2.5 | 2.2 | .6 |
| 185 | 10.1 | 4.5 | 5.5 | 2.1 |
| 190 | 2.0 | 1.6 | | |
| 191 | 2.0 | 1.6 | | |
| 192 | 3.2 | 2.5 | | |
| 193 | 1.6 | 1.2 | | |
| 196 | 1.4 | 1.1 | | |
| 197 | 1.2 | 1.0 | | |

*insulin analogue = A14E, B25H, B29K(N(eps)Octadecanedioyl-γGlu-OEG-OEG), desB30 human insulin
^{#}GLP-1 analogue = N-epsilon26-[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl]-[Aib8,Arg34]GLP-1-(7-37)

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. An N-terminally acylated peptide or oligopeptide having the structure
Cx-Aaa10-Aaa9-Aaa8-Aaa7-Aaa6-Aaa5-Aaa4-Aaa3-Aaa2-Aaa1-OH (SEQ ID No: 1, Chem I)
where Cx is a fatty acid with a length between 6 and 20 carbons, and
wherein Aaa1 is an aromatic amino acid; Aaa2 is any amino acid except Lys or Asp; Aaa3 is any amino acid; Aaa4-10 is any amino acid or absent.

2. An N-terminally acylated peptide or oligopeptide according to claim 1 wherein Aaa1 is Tyr, Trp or Phe.

3. An N-terminally acylated peptide or oligopeptide according to claim 1 wherein Aaa2 is Pro or Leu.

4. An N-terminally acylated peptide or oligopeptide according to claim 1 wherein Aaa3 is Arg, Lys, His, Trp, Tyr or Phe.

5. An N-terminally acylated peptide or oligopeptide according to claim 1 wherein Aaa10 is Leu, Thr, Lys, Arg or His.

6. An N-terminally acylated peptide or oligopeptide according to claim 1 wherein Aaa10 is Lys, Arg or His.

7. An N-terminally acylated peptide or oligopeptide according to claim 1 wherein Aaa6-9 are absent.

8. An N-terminally acylated peptide or oligopeptide according to claim 1, wherein Aaa2 is OEG ([2-(2-aminoethoxy)ethoxy]ethylcarbonyl) or γGlu or βAsp.

9. An N-terminally acylated peptide or oligopeptide according to claim 1, wherein Aaa3 is OEG or γGlu or βAsp.

10. An N-terminally acylated peptide or oligopeptide according to claim 1, wherein Aaa4 is OEG or γGlu or βAsp.

11. An N-terminally acylated peptide or oligopeptide according to claim 1, wherein the length of the fatty acid is between 12-16.

12. An N-terminally acylated peptide or oligopeptide according to claim 1, which is an inhibitor of proteolytic activity in an extract from the gastrointestinal tract (GI tract).

13. An N-terminally acylated peptide or oligopeptide according to claim 1, which is an inhibitor of proteolytic activity such as proteolytic activity of trypsin, chymotrypsin, elastase, carboxypeptidase and/or aminopeptidase.

14. An oral pharmaceutical composition according to claim 1 further comprising a pharmaceutically active ingredient which is a peptide or protein.

15. An oral pharmaceutical composition according to claim 14, which is a liquid composition.


SEQUENCE LISTING (1 sequence)

SEQ ID NO: 1 - protein, 11 aa, Artificial Sequence
  Synthetic
XAAAAAAAAPF